(19) United States
(12) Patent Application Publication
(10) Pub. No.: US 2024/0277370 A1
(43) Pub. Date: Aug. 22, 2024

(54) SURGICAL CUTTING INSTRUMENT, ROTATIONAL JOINT AND METHOD, PARTICULARLY FOR ROBOTIC SURGERY AND/OR MICRO-SURGERY

(71) Applicant: MEDICAL MICROINSTRUMENTS, INC., Wilmington, DE (US)

(72) Inventors: Giorgio LAZZARI, Pisa (IT); Neri PIEROTTI, Pisa (IT); Marco BACCHERETI, Pisa (IT); Massimiliano SIMI, Pisa (IT); Nicola PINESCHI, Pisa (IT)

(21) Appl. No.: 18/570,510

(22) PCT Filed: Jun. 16, 2022
(86) PCT No.: PCT/IB2022/055598

§ 371 Date: Dec. 14, 2023

(30) Foreign Application Priority Data
Jun. 21, 2021 (IT) 102021000016175

(51) Int. Cl. A61B 17/295; A61B 17/3201; A61B 34/37

(52) U.S. Cl. CPC A61B 17/295; A61B 17/3201; A61B 34/37

(57) ABSTRACT

A surgical instrument includes an articulated end-effector including a support structure, first and second tips having a respective elongated body including a proximal attachment root and a distal free end. The first and second roots are axially adjacent and globally interposed with the support structure. The support structure, and first and second tips are mutually articulated in a common rotation axis defining an axial direction coincident/parallel to the common rotation axis, defining a relative degree of freedom of opening/closing between the first and second tips. The first tip body includes a blade portion with a cutting edge rotating with the first free end. The second tip includes a counter-blade portion rotating with the second free end. The counter-blade portion abuts the cutting edge, elastically axially bending the blade portion. The first and second roots each directly and intimately contact the support structure.

### FIELD OF THE INVENTION

The present invention relates to a surgical instrument capable of performing a cutting action.

The surgical instrument according to the invention is particularly suitable but not uniquely intended for applications in teleoperated robotic microsurgery.

The present invention further relates to a rotational joint of a cutting joint of a surgical instrument.

The present invention further relates to a robotic surgery system comprising at least one surgical instrument.

Furthermore, the present invention relates to a manufacturing method as well as to a manufacturing fixture as well as to a manufacturing semi-finished product.

The manufacturing method is particularly suitable for making one or more blades for a surgical instrument.

The present invention further relates to a method for performing a cutting action.

### Background Art

Robotic surgery apparatuses are generally known in the art and typically comprise a central robotic tower (or cart) and one or more robotic arms extending from the central robotic tower. Each arm comprises a motorized positioning system (or manipulator) for moving a surgical instrument distally attachable thereto, in order to perform surgical procedures on a patient. The patient typically lies on an operating bed located in the operating room, in which sterility is ensured to avoid bacterial contamination due to non-sterile parts of the robotic apparatus.

In the context of traditional, i.e., non-robotic, surgery, instruments of the needle-driver/sutures-cutter type are generally known, which typically comprise at the opposite end of the maneuvering rings a needle-driver/sutures-cutter formed by the two free ends having gripping surfaces for the surgical needle and blades for cutting the suture. In some cases, the blades are made in a seat or recess made in the body of the gripper that is accessible through a distinct and separate access opening with respect to the opening for accessing the gripping surfaces for the needle.

Surgical scissors are also known in the field, which comprise at the opposite end of the operating rings two opposite blades on the free ends. A spring can be provided for the maneuvering rings. Typically, the opening angle of the free ends useful to perform the cutting action in such traditional surgical scissors must be less than 25°.

Furthermore, in the field of robotic surgery, end-effector solutions of the needle-driver/sutures-cutter type for laparoscopy have been suggested, having opposite gripping surfaces and respective blades placed at the distal end of an elongated shaft. Typically, the blade is co-molded with the respective gripping surface for the needle forming a cantilevered protrusion with respect to the gripping surface and placed proximally thereto, i.e., between the gripping surface and the pivot hinge of the gripping surfaces. Therefore, a single molded piece usually comprises a root for forming a part of the hinge, a free end, a gripping surface and a blade which extends with respect to the gripping surface in the closing direction towards the opposite and faceable other blade of the end-effector of the needle-driver/sutures-cutter type.

Scissor-type end-effector solutions for robotic surgery have also been suggested, in which each free end of the end-effector is provided with a blade, as shown in US 2008/0119870, for example.

Both in the surgical instruments for robotic surgery of the needle-driver/sutures-cutter type, and in those of the scissor type, a plurality of elastic washers of the “Belleville washer” type ensure a preload between the roots of the two pieces forming the end-effector to determine in closing a mechanical interference condition between the blades aimed at making the cut. Therefore, when the end-effector closes, the opposite blades enter interference and cause a transverse sliding away between the respective roots, counteracting the elastic influence action exerted by said elastic Belleville washers to the hinge.

Otherwise, US-2019/0105032 shows an end-effector needle-driver/sutures-cutter end-effector, in which the blades each comprise in a single piece an elastic cantilevered tab, said two elastic cantilevered tabs extending in a direction parallel to the pin towards each other, so that the elastic preload is given by the contact between the two cantilevered tabs. Thereby, assembling Belleville-type elastic washers on the hinge is avoided, thus allowing an axial space to be left at the hinge between the two blades to accommodate the sliding thereof relative to the variation of the elastic reaction exerted by the cantilevered elastic tabs thereof in mutual contact.

Another known example is given by US-2020/0107894 which shows a needle-driver/sutures-cutter solution in which the blade is housed in a longitudinal pocket of the gripping link and is rotatable independently with respect thereto, so that it can be extracted if necessary.

Otherwise, an example of a surgical scissor is shown by US-2016/0175060 which discloses an interchangeable tip solution, i.e., having the distal cutting joint separable when in operating condition. Furthermore, such a known solution uses elastic cutting blades both curved in the same transverse direction to obtain a preload between the blades given by the shape and elastic properties thereof.

A further known example of a surgical instrument scissor for robotic surgery is disclosed in US-2019/0282291.

Alternatively or in addition to the plurality of washers of the “Belleville washer” type, an adjustment screw can be provided at the hinge in order to adjust the cutting interference between the blades, usually forming the articulation pin itself. If the adjustment screw is provided in combination with the plurality of elastic washers of the “Belleville washer” type, it works by counteracting the elastic action of the springs to allow an end of adjustment in elastic preload.

Typically, the known surgical scissors attributable to the types described above have two blades both curved axially in the same direction to ensure a mutual contact of cutting interference which are adjusted so that they are capable of satisfactorily cutting only for small opening angles, for example not exceeding 25°, i.e., the blades cut well only close to or at the distal free end where the axial curvature (i.e., in the direction of the hinge axis) is more accentuated, while in the respective proximal sections thereof they are axially spaced apart and thus unsuitable for performing a precise cut (the tissue to be cut bends between the blades without separating). Conversely, if the blades are adjusted to be in mechanical cutting interference contact in the proximal portions thereof, i.e., for high opening angles, for example greater than 15°, they will be unsuitable for completely closing because the distal curvature thereof will in fact create closing stroke ends precluding the cutting capacity for small opening angles. Strongly increasing the tightening force of the blades, they could close but would necessarily axially distance themselves again in the proximal section thereof, losing the cutting capacity in the proximal area. For these reasons it is usually chosen to tighten the adjustment screws of the blades of the known surgical scissors so that a mechanical interference condition can be reached only close to the free ends, as they are easier to view and require a lower degree of opening, thus a smaller footprint.

The miniaturization of surgical instruments and in particular of the ends or end-effectors thereof for robotic surgery is particularly desirable because it opens up advantageous scenarios of minimal invasiveness for the patient undergoing surgery, as well as the millimeter and sub-millimeter dissection capacity of tissues.

The known solutions of the type mentioned above are unsuitable for a boosted miniaturization because they would impose impossible processes for the production of the pieces as well as complicated assembly strategies of the pieces to obtain the assembled end-effector. For example, consider the need to assemble micro-parts to the hinge while counteracting the elastic reaction of Belleville-type elastic washers, as well as the objective extreme difficulty of manufacturing by co-molding micro-ridges and micro-undercuts which must be sufficiently robust to withstand rather high stresses when in operation and at the same time geometrically shaped to minimize frictions. In fact, as is well known, at the micro-scale surface forces such as friction are dominant over volume forces.

Furthermore, in surgical instruments having cutting end-effectors actuated by actuation cables or tendons, to ensure a high closing force such as to exert a precise cutting action without damaging the actuation tendons it is typically, necessary to make a reducer, i.e., a pulley of relatively large diameter, but this limits the miniaturization of the pieces especially close to the distal end of the end-effector. Otherwise, to maintain the size of the end-effector compact, it would be necessary to increase the tensile strength of the actuation tendons at the expense of the longitudinal bendability thereof and thus in any case imposing a distal pulley of relatively large diameter; or an attempt could be made to reinforce the tendons by increasing the diameter thereof, but as is apparent to those skilled in the art, both of these choices would be an obstacle severely hindering miniaturization.

Furthermore, as the scale decreases, it becomes increasingly complex to precisely size elements intended to form when rotational joints are assembled, such as end-effector gripping terminals of a surgical instrument, because small machining uncertainties at the level of the fulcrum, i.e., the hinge, impose enormous inaccuracies close to the respective cantilevered free ends and therefore at the cutting blades in the case of scissor-type instruments or at the gripping surfaces in the case of tools such as needle-driver/sutures-cutters.

Similarly, therefore, in an attempt to transmit a high closing force such as to exert a precise cutting action without damaging the actuation tendons, the provision of leverages associated with the blades (a solution in itself known in the art) would also be an obstacle to miniaturization, even for the sole objective difficulty of making the pieces on such a small scale that they simultaneously prove to be robust when in working conditions, as well as for the footprints in the area proximal to the common rotation axis of the free ends, as well as for the difficulty of assembly.

The end-effector portions which are placed distally with respect to the hinge, whether only the cutting blades or the cutting blades and gripping surfaces, are typically designed to perform extremely precise tasks and at the same time the cutting blades must ensure a precise and clean cutting action.

U.S. Pat. No. 10,864,051, WO-2017-064301, WO-2019-220407, WO-2019-220408, WO-2019-220409 and US-2021-059776 to the same Applicant disclose teleoperated robotic surgery systems having one or more surgical instruments controlled by one or more master interfaces. Furthermore, U.S. Pat. No. 10,582,975, EP-3586780, WO-2017-064303, WO-2018-189721, WO-2018-189729, US-2020-0170727 and US-2020-0170726 to the same Applicant disclose various embodiments of surgical instruments suitable for robotic surgery and microsurgery. These types of surgical instruments typically comprise a proximal interface portion having an interface intended to be driven by a robotic manipulator, a shaft, and an articulated cuff at the distal end of the shaft. The articulated cuff consists of a plurality of links moved by a plurality of tendons (or actuation cables). Two end tip links have a free end and a degree of freedom of opening/closing therebetween and can be adapted to handle a needle as well as a suture thread forming an end-effector of the needle-holder gripper type for teleoperated robotic surgery to perform anastomosis or other surgical therapies.

Furthermore, WO-2017-064305, EP-3362218 and EP-3597340 to the same Applicant disclose methodologies for manufacturing a surgical instrument including wire electro-erosion, also known with the terminology “WEDM”, “wire-cut”, “electro-erosion”, “spark-machining”, or “spark-eroding”.

For example, WO-2017-064306 to the same Applicant shows a surgical instrument in which the tendons for actuating the degree of freedom of opening/closing of the articulated end-effector slide on convex ruled sliding surfaces of the end-effector links, simultaneously avoiding routing the tendons inside guide grooves or channels with concave section. Thereby, the cross-section of the sliding contact portion between the tendons and the link is minimized, thus reducing the sliding 10 friction and allowing a boosted miniaturization of the articulated end-effector while ensuring a high dexterity given by the end-effector joints, such as rotational joints of pitch and yaw.

Furthermore, WO-2018-189722 to the same applicant discloses a surgical instrument in which the tendons for actuating the degree of freedom of opening/closing of the articulated end-effector, in addition to sliding on convex ruled sliding surfaces of the end-effector links, similar to what was previously discussed, are wound on said convex ruled sliding surfaces, describing arcuate paths which underlie a particularly high winding angle. In fact, by virtue of the low sliding friction of the tendons, they are capable of remaining in contact with the convex ruled surface of a link for a relatively long and arcuate longitudinal section.

In addition, US-2021-0106393 to the same applicant discloses some embodiments of a tendon consisting of intertwined polymer fibers. The use of polymer tendons allows reducing the sliding friction with respect to the use of metal tendons and at the same time an adequate dimensioning of the tendon allows traveling winding longitudinal paths in the articulated end-effector.

Therefore, the need is strongly felt to provide a surgical instrument solution which is suitable for extreme miniaturization and at the same time robust, reliable and capable of providing a precise and repeatable cutting action.

Furthermore, the need is felt to suggest a surgical instrument solution for teleoperated robotic micro-surgery which is simple to produce and assemble and to build as well as reliable and precise and robust when under operating conditions, is adapted to allow a desired and controlled spatial orientation of the cutting action with respect to, for example, the main longitudinal extension direction of the surgical instrument body which can be useful to facilitate the observation of the surgery.

The need is felt to suggest a solution which allows assembling an articulated tip micro-instrument provided with grip and/or scissors and which consists of the smallest number of components so that it can be assembled easily and in a cost-affordable manner without imposing a reduced dexterity of the articulated end-effector.

The need is felt to suggest a solution which allows making micromechanical parts and in particular sharpened micromechanical parts with a high geometric precision and repeatability for the formation of an articulated tip micro-instrument provided with grip and/or scissors.

In addition, in the medical-surgical field, the need is felt to provide a manufacturing process solution capable of making one or more miniaturized blades for making a miniaturized surgical cutting tool. In particular, the need is felt to provide a robust, repeatable and serializable manufacturing process capable of producing one or more miniaturized blades in an economically sustainable manner for single-use surgical instrumentation.

### Solution

It is an object of the present invention to obviate the drawbacks complained of with reference to the background art.

This and other objects are achieved by a surgical instrument according to claim 1, as well as by a robotic surgery system according to claim 15, as well as by a rotational joint according to claim 16.

Some advantageous embodiments are the subject of the dependent claims.

According to an aspect of the invention, a surgical instrument is provided comprising an articulated end-effector.

The articulated end-effector (or articulated end device) can be mounted at the distal end of a shaft or rod of the surgical instrument. The articulated end-effector is preferably actuated by actuation tendons.

The articulated end-effector comprises a support structure, a first tip having an elongated body comprising a first proximal attachment root and a first distal end, and a second tip having an elongated body comprising a second proximal attachment root and a second distal end. The distal ends of the tips are preferably free ends, although a constraint, for example a hinge and/or a rail, can be provided at the distal end of one or both of the tips.

The support structure, the first proximal attachment root and the second proximal attachment root are mutually articulated defining a degree of freedom of opening/closing between the first and second free ends.

The first tip comprises a blade portion with a cutting edge integral in rotation with the first free end. The blade portion is elastically bendable in the axial direction.

The second tip comprises a counter-blade portion integral in rotation with the second free end.

The counter-blade portion is adapted to abut against said cutting edge by axially elastically bending said blade portion, so that said cutting edge of the first tip and said counter-blade portion of the second tip reach a mechanical interference contact condition to exert a cutting action.

The support structure, the first proximal attachment root and the second proximal attachment root form a rotational joint of a cutting joint. Said distal rotational joint can be a rigid rotational joint in the axial direction, in which no elastic elements are provided in the coupling and the elasticity is provided distally with respect to the rotational joint, i.e., on the blade.

Preferably but not necessarily said support structure comprises two prongs. The support structure can belong to a support link which is made in a single piece.

The first tip as well as the second tip can be made in a single piece forming a link or they can be formed by assembling several links, for example two links. In accordance with an embodiment, the first tip is formed by a blade link and a blade holder link integral with each other in rotation. In accordance with an embodiment, the second tip is made in a single piece forming a second tip link or reaction link.

According to an embodiment, the first root of the first tip is in direct and intimate contact with the support structure, for example with the first prong of the support structure, and the second root of the second tip is in direct and intimate contact with the support structure, for example with the second prong of the support structure.

The support structure is preferably a rigid structure, for example it is free of elastic preload elements between the prongs.

According to an embodiment, the first root of the first tip and the second root of the second tip are axially next to each other.

The first root of the first tip and the second root of the second tip can be globally interposed in the support structure, for example interposed between the prongs of the support structure.

According to an embodiment, the support structure, the first tip and the second tip are articulated to each other in a common rotation axis defining an axial direction coincident with or parallel to the common rotation axis.

According to an embodiment, the first root of the first tip and the second root of the second tip are articulated with respect to the support structure about said common rotation axis, defining a degree of freedom of orientation between the support structure and the assembly formed by said first tip and said second tip.

According to an embodiment, the first root of the first tip and the second root of the second tip are articulated to each other about said common rotation axis, defining a relative degree of freedom of opening/closing between the first tip and the second tip.

According to an embodiment, the axial elasticity necessary to perform the cutting action is provided by the blade portion and axially the roots are packed with the support structure, making a reaction to the elastic bending of the blade, preventing axial displacements from occurring between the roots.

According to an embodiment, said first root of the first tip comprises a first axially facing external contact surface and the first prong of the support structure comprises a first axially facing internal contact counter-surface, said second root of the second tip comprises a second axially facing external contact surface and the second prong of the support structure comprises a second axially facing internal contact counter-surface. Said first external contact surface of the first root, said first internal contact counter-surface of the first prong, said second external contact surface of the second root, and said second internal contact counter-surface of the second prong can all be parallel to one another.

The counter-blade portion of the second tip can project axially to bend the first tip. Preferably said counter-blade portion is a curved protruding surface having a concavity facing axially inwards.

The body of the counter-blade portion of the second tip can be elastically bendable in the axial direction, preferably axially outwards. Thereby, the axial elasticity necessary to perform the cutting action is provided by the blade portion and the counter-blade portion, jointly or separately for example depending on the opening angle of the tips. According to an embodiment, the body of the second tip comprises a proximal cantilevered arm being elastically deformable in an external axial direction and having a proximal free end and a proximal portion of the counter-blade portion belonging to said proximal cantilevered arm.

By virtue of the suggested solutions, the surgical instrument can be capable of performing a cutting action for opening angles of the degree of freedom of opening/closing up to 60°.

The sharpening of the blade portion can be performed by wire electro-erosion (WEDM). Therefore, the cutting edge of the blade portion can be made sharpened and cutting by a wire electro-erosion process.

At least one of the first tip and the second tip can comprise an axial deformation seat forming an axial recess for housing the elastic deformation of the blade portion and/or the counter-blade portion during the cutting action.

Preferably, said first root of the first tip comprises a first through hole, and said second root of the second tip comprises a second through hole which are all circular through holes and coaxial to said common rotation axis. Said holes can receive a single articulation pin.

The body of the first tip can be formed by two separate pieces, or links, comprising a blade link having a body comprising in a single piece said blade portion with said cutting edge and a blade link root, and a blade holder link having a blade holder link root. In such a case, the blade link root and the blade holder link root are next to and in direct and intimate contact with each other, jointly forming said first root of the first tip. In such a case, a rotational drag engagement is provided between said blade link and said blade holder link of the first tip which can be placed distally with respect to the first root of the first tip, and is preferably placed along the longitudinal extension of the blade portion. A closing stroke end can be provided for said blade link which is placed distally with respect to the first root of the first tip. In such a case, the blade link root can be axially interposed between said blade holder link root and the second root of the second tip and in direct and intimate contact therewith.

According to an embodiment, the first root of the first tip comprises, integral in rotation with said blade portion, a first termination seat for at least one actuation tendon of the first tip about said common rotation axis, and the second root of the second tip comprises, integral in rotation with said counter-blade portion, at least a second termination seat for at least one actuation tendon of the second tip about said common rotation axis.

Said support link articulated about a proximal rotation axis can comprise in a single piece at least a third termination seat for at least one actuation tendon of the support link about said proximal rotation axis.

The support structure can have a body comprising in a single piece one or more convex ruled surfaces of support links with parallel generatrices and a distal connection portion which can comprise two prongs.

According to an embodiment, the articulated end-effector comprises a connection link connected to the distal end of the rod having a body comprising in a single piece one or more convex ruled surfaces of connection links with parallel generatrices, and a first distal connection portion connected with a proximal connection portion of the support link, defining a proximal rotational joint for the connection link and the support link so that they can rotate relatively about a common proximal rotation axis.

The articulated end-effector can comprise a first tip, for example a blade holder link, articulated to the support link having a proximal attachment root having a body comprising in a single piece a pulley portion formed by one or more convex ruled surfaces with parallel generatrices.

A drag portion can be provided in a single piece with said proximal attachment root to make said root integral in rotation with a blade portion, where the blade portion is made in a separate piece. In fact, the articulated end-effector can comprise a blade link, integral in rotation with said blade holder link of the first tip, having a body comprising in a single piece a cutting edge and a drag counter-portion engaged with said drag portion of the attachment root.

The articulated end-effector can comprise a second tip, for example comprising a reaction link, articulated to the support link and to the assembly formed by the blade link and the blade holder link, having a body comprising in a single piece an attachment root having a pulley portion formed by one or more convex ruled surfaces with parallel generatrices.

According to an embodiment, the first attachment root and the second attachment root define with the distal connection portion of the support structure a distal rotational joint defining a common distal rotation axis for a cutting joint.

According to an embodiment, a first pair of antagonistic tendons is connected to the first attachment root, for example the blade holder link root, to move the cutting edge about said common distal rotation axis, and a second pair of antagonistic tendons is connected to the second root to move the counter-blade portion about said common distal rotation axis.

According to an embodiment, the first attachment root, for example the blade holder link root, comprises in a single piece at least a first termination seat which receives said first pair of antagonistic tendons and the second attachment root comprises in a single piece at least a second termination seat which receives said second pair of antagonistic tendons.

Said one or more convex ruled surfaces with parallel generatrices of the link can be parallel to said common proximal rotation axis.

Preferably, at least one of said one or more convex ruled surfaces with parallel generatrices of the support link is parallel to said common proximal rotation axis.

Preferably, said one or more convex ruled surfaces of the blade holder root with parallel generatrices of the first root and said one or more convex ruled surfaces with parallel generatrices of the second root are parallel to the common distal rotation axis.

The first pair of antagonistic tendons and the second pair of antagonistic tendons are adapted to slide longitudinally on said one or more convex ruled surfaces of the connection link if provided and on said one or more convex ruled surfaces of the support link and are adapted to wind/unwind without sliding on the respective convex ruled surface of the blade holder link root, i.e., the first root or the reaction link, i.e., the second root, to move the blade link and the counter-blade portion in opening/closing, respectively.

A first distance in a direction parallel to the common distal rotation axis can be identified between the first termination seat of the first root and a surface of said one or more convex ruled surfaces of the support structure, for example of the support link, which is constant for any cutting condition.

A second distance in a direction parallel to the common distal rotation axis can be identified between the second termination seat of the second root and a surface of said one or more convex ruled surfaces of the support structure, for example of the support link, which is constant for any cutting condition.

In accordance with an embodiment, a first cantilevered drag leg extends from the first root forming a free end of the first leg, axially delimiting said first termination seat, and a second cantilevered drag leg extends from the second root forming a free end of the second leg, axially delimiting said second termination seat, said first and second cantilevered legs each comprising abutment and drag walls placed as an undercut with respect to the respective termination seats acting as dragging abutments for the respective tendon termination. In such a case, it is possible to identify a first distance in an axial direction between the first cantilevered leg and a surface of said one or more convex ruled surfaces of the support structure, for example of the support link, is constant for any cutting condition and a second distance in a direction parallel to the common distal rotation axis between the second cantilevered leg and a surface of said one or more convex ruled surfaces of the support structure, for example of the support link, is constant for any cutting condition.

The first distance and the second distance can be mutually equal.

The first distance and/or the second distance can be zero.

The first attachment root can comprise a first surface facing axially outwards, and the second root can comprise a second surface facing axially outwards, and in which a further distance in the axial direction can be identified between said first surface and said second surface which is constant for any cutting condition.

According to an embodiment, the overall sliding friction force exchanged between each tendon and all the ruled surfaces of the links on which the tendon slides, when in operating conditions, is much less than the tensile force transmitted by the same tendon to achieve the elastic bending deformation of the blade portion when the degree of freedom of opening/closing is moved in closing to exert a cutting action. In other words, said sliding friction force of the tendons can be much less than the mechanical interference contact friction force between the blade and the counter-blade. For this purpose, the tendons can be made of polymer material, and the links can be made of metallic material, and the convex ruled surfaces with parallel generatrices of the links can be smooth, to reduce the longitudinal sliding friction of the tendons on the links. For example, the ruled surfaces of the links are obtained by wire electro-erosion.

Preferably, all the convex ruled surfaces of the connection link, the support link, the pulley portion of the first root and the pulley portion of the second root lack longitudinal channels. Therefore, the actuation tendons do not slide inside concave channels.

A third pair of antagonistic tendons can be provided for moving the support link about said common proximal rotation axis with respect to the connection link, the support link comprising at least a third termination seat which receives the tendon terminations of said third pair of antagonistic tendons. Preferably, the actuation tendons of the support link of said third pair of antagonistic tendons wind/unwind without sliding longitudinally on said one or more convex ruled surfaces of the support link, which therefore act as pulley surfaces for the actuation tendons of the third pair of antagonistic tendons.

According to an aspect of the invention, a cutting method for a surgical instrument comprises the step of providing an articulated end-effector at the distal end of a rod or shaft comprising a support structure, a first tip and a second tip.

The method comprises the steps of longitudinally sliding the actuation tendons of at least one pair of antagonistic tendons on one or more convex ruled surfaces with parallel generatrices of the support structure to orient the cutting edge of the blade link in a desired orientation, and make the actuation tendons of at least one pair of antagonistic actuation tendons of the distal rotational joint longitudinally slide on one or more ruled convex surfaces with parallel generatrices of the support structure to bring the cutting edge into contact with said counter-blade portion.

The method further comprises elastically bending at least one of the cutting edge and the counter-blade portion, making a mechanical interference contact therebetween, exerting a cutting action.

A connection link can be provided, having convex ruled surfaces parallel to the proximal rotation axis on which all of the actuation tendons of the support link, of the first tip and of the second tip slide. The step of longitudinally sliding the antagonistic tendons of at least one pair of antagonistic actuation tendons of the distal rotational joint on the convex ruled surfaces with parallel generatrices of the connection link and the support link, can comprise the step of winding at least one movement tendon of the distal rotational joint on the convex ruled surfaces on which it slides, by a winding angle between 60° and 300°, and preferably greater than 120°.

According to an aspect of the invention, a rotational joint of a cutting joint comprises: a distal connection portion of a support structure, an attachment root integral in rotation with a blade having a cutting edge and having an axially elastically bendable body, an attachment root integral in rotation with a counter-blade portion, in which the cutting edge of the blade link is adapted to abut against said counter-blade portion during the movement of the degree of freedom of opening/closing in a mechanical interference contact condition to exert a cutting action.

The blade and counter-blade are preferably integral in rotation with respective distal free ends relatively movable according to the degree of freedom of opening/closing. Preferably, the free ends are also globally orientable with respect to the support structure about the rotation axis of the rotational joint.

The blade is preferably axially elastically bendable, so as to confer the axial elasticity to the cutting action, while said rotational joint is rigid in the axial direction, i.e., relative movements between the roots as well as between the roots and the support structure are avoided.

The cutting joint is preferably a distal joint of an articulated end-effector, in which a first free end integral in rotation with the blade and a second free end integral in rotation with the counter-blade are included.

By virtue of the suggested solutions, extreme and boosted miniaturization of an articulated end-effector is allowed, for example which reproduces a wrist, without pulleys which are replaced by surfaces ruled in one piece with links, having a very small radius. Therefore, the known metal tendons can be replaced by miniaturized polymer tendons which, by virtue of the low friction, slide on such ruled surfaces defining the movement thereof,

It is possible to make surgical cutting instruments of minimum size having a simplified opening-closing and cutting mechanism, replacing an adjustment dowel and/or a Belleville spring train with the inclusion of an elastic blade (and preferably a curved counter-blade) the closure with interference of which exerts the deformation and the cutting action thereof.

Those components (keyed pulleys or rotationally connected to the links, Belleville-type springs on the distal joint pin, blade adjustment screws, metal actuation tendons) which are relatively bulky and/or difficult to assemble as the scale descends, with consequent risks of intolerable clearance, which would represent an obstacle to miniaturization, are in fact eliminated.

In accordance with an embodiment, the attachment root of the first tip and/or the second tip, having a convex ruled winding surface for the respective tendon forming a pulley portion without longitudinal channels, comprises geometric drag elements adapted to allow the interlocking of a further component, which is preferably a planar and elastic blade, and such geometric elements are such as to guide the blade integrally against the counter-blade in the opening and closing action.

At least the blade portion can be made by wire electro-erosion.

According to an aspect of the invention, a method for making one or more blades by wire electro-erosion comprises the steps of: (i) providing a wire electro-erosion machine having a cutting wire and providing a fixture mounted on the wire electro-erosion machine; (ii) mounting at least one workpiece to the fixture; (iii) sharpening at least one edge to be sharpened of the at least one workpiece by performing with the cutting wire a sharpening through cut on the at least one workpiece. The sharpening step carries out a sharpening process to obtain said cutting edge of the blade portion.

According to an aspect of the invention, a method for making one or more blades by wire electro-erosion comprises the steps of: (i) providing a wire electro-erosion machine having a cutting wire and providing a fixture mounted to the wire electro-erosion machine, in which the fixture is mounted so that at least one portion thereof can rotate about a rotation axis which is transverse to the longitudinal extension of the cutting wire; (ii) mounting at least one workpiece to the fixture; (iii) sharpening at least one edge to be sharpened of the at least one workpiece by performing with the cutting wire a sharpening through cut on the at least one workpiece; (iv) shaping the at least one workpiece by performing with the cutting wire of a shaping through cut on the at least one workpiece.

Between the sharpening step and the shaping step, the further step is performed of rotating the at least one portion of the fixture about the rotation axis thereof by a sharpening rotation angle other than 90°.

By virtue of such a method it is possible to make one or more blade portions. In an embodiment, by virtue of such a method it is possible to make one or more blade links.

Such a sharpening rotation angle can be identical to the angle formed in the cross-section of the cutting edge made on the workpiece.

By virtue of such a method, replacements of the at least one workpiece on the fixture are avoided.

The method can make a plurality of blades on the same workpiece in which the sharpening and shaping steps are the same for all the blades of said plurality. The sharpening step can be carried out by a single cutting trajectory (or a single cutting path) having a start point and an end point which determines the sharpening of a plurality of edges to be sharpened. The shaping step can be carried out by a single cutting trajectory (or a single cutting path) having a start point and an end point which determines the shaping of a plurality of pieces to be machined.

The workpiece can comprise a plate-like body, such as a plate, a strip, a belt, and the sharpening and shaping steps each include making a through cut through the thickness of the plate-like body of the workpiece. The thickness of the plate-like body can be less than 1 millimeter, such as between 0.05 and 0.5 millimeters. The plate-like body can be an elastic body which can be elastically deformable by bending, for example made of steel for blades.

The shaping step can comprise making at least one hole edge intended to delimit a through hole through the thickness of the blade link 30, for example said through hole can be a centering hole, in which the hole edge can have an open profile defining a cutting channel on the body of the piece due to the passage of the cutting wire.

The mounting step can comprise assembling to the fixture a plurality of workpieces, in which the sharpening and shaping steps are performed by individually sharpening and shaping each workpiece of said plurality.

The fixture can be made so that the individual pieces to be machined can be machined individually by the cutting wire on at least two cutting planes misaligned from each other by said sharpening rotation angle. In other words, the workpieces to be machined can be mounted to the fixture so that the cutting edge, which extends substantially straight, intersects at most one of the workpieces to be machined at a time on each cutting plane provided.

The fixture can include fixing multiple planar elements (strips) which are individually machinable by wire electro-erosion in one or more rotation configurations about the rotation axis.

After the shaping step, a step of reshaping the workpiece can be included on a second, different cutting plane by performing a second shaping through cut on the workpiece, in which between the shaping step and the reshaping step the fixture has completed a rotation which can be substantially equal to 90°. The sharpening step can be carried out between the shaping step and the reshaping step. The reshaping step can be performed on a sub-group of workpieces.

A zeroing and calibration strategy of the electro-erosion machine can be included, which includes identifying a point of origin by contacting a known reference on the fixture and/or workpiece with the cutting wire. According to an implementation, the method comprises the further steps of identifying a point of origin or reference of the cutting path and approaching, for example until reaching, the point of origin or reference with the cutting wire. The point of origin can belong to the workpiece, such as the edge of the workpiece to be sharpened.

The point of origin or reference can be a single point of origin for both the sharpening step and the shaping step, as well as for the reshaping step, and the control system of the wire electro-erosion machine can store said single point of origin or reference and relate it geometrically (e.g., trigonometrically) to the kinematic rotation of the fixture of said sharpening rotation angle to process the next cutting path. The sharpening cut and the shaping cut can both start from the same point which is in geometric relation to the point of origin or reference. After the identification step and before the sharpening and/or shaping step, it is possible to perform a rotation of the fixture about the rotation axis by a certain angle which can be an acute angle.

The sharpening through cut can be performed with repeated multiple passes of the cutting wire along a same sharpening cutting path, and the number of said repeated multiple passes of the cutting wire to perform said sharpening through cut is greater than the number of passes made to perform the shaping through cut.

The sharpening of the cutting edge 34 carried out can be a “no back bevel” or “chisel edge” type sharpening.

The shaping step can include not separating the blades and leaving at least one bridge of material for each blade intact.

According to an aspect of the invention, a semi-finished product is provided, comprising a plate-like body, for example a sheet-like body, having in a single piece a plurality of blades, for example blade links, shaped and connected to one another by connection bridges.

The fixture can comprise a plurality of seats for receiving workpieces.

A plurality of links of the articulated end-effector which also includes said blade portion (e.g., when made on a blade link) can be made by wire electro-erosion.

According to an aspect of the invention, a method for manufacturing a articulated surgical cutting instrument by wire electro-erosion comprises the following steps of: (i) providing a wire electro-erosion machine comprising a cutting wire and a fixture which is rotatable with respect to the cutting wire about a rotation axis which is transverse to the longitudinal extension of the cutting wire; (ii) assembling a plurality of workpieces to be machined on the fixture; (iii) sharpening at least one edge to be sharpened of at least one workpiece of said plurality by performing with the cutting wire a sharpening through cut on the at least one workpiece; (iv) shaping on a first cutting plane at least some of, but also all, the workpieces of said plurality one at a time; (v) reshaping on a second cutting plane at least some, but also all, of the workpieces of said plurality by performing a shaping through cut with the cutting wire on said at least some of, but also all, the workpieces of said plurality one at a time, in succession.

Between the sharpening step and the shaping step on a first cutting plane, the step of rotating the fixture by a sharpening rotation angle different from 90° is performed. In other words, the sharpening step and the shaping step on a first cutting plane, the fixture has completed a rotation of a sharpening angle other than 90°.

Between the shaping step on a first cutting plane and the reshaping step on a second cutting plane, the step of rotating the fixture about the rotation axis thereof by a rotation angle preferably substantially equal to 90° is included.

At least one of the workpieces of said plurality can be a small cylinder of material.

At least one of the workpieces of said plurality can be a plate-like body, for example a strip or ribbon or plate.

The arrangement of the workpieces of said plurality of workpieces on the jig preferably must meet the condition that the cutting wire intersects at most one of the workpieces at a time in each cutting step (i.e., sharpening, shaping and reshaping).

The method can comprise the step of separating the shaped pieces.

The method can comprise the step of assembling the separate pieces together, in which at least one of the pieces has a cutting edge.

According to an aspect of the invention, a fixture (or jig) is provided for an electro-erosion machine having a fixing portion to the machine and a housing portion for receiving at least one workpiece, in which the housing portion is rotatable with respect to the fixing portion. A motor can be provided for performing the rotation.

The fixture can receive a plurality of workpieces and the machine can process the pieces of said plurality individually on at least two cutting planes, in which at least one cutting profile is for shaping.

The fixture is configured so as to arrange the workpieces in respective housing seats so that the cutting edge intersects one workpiece at a time on at least two cutting planes. The fixture can be configured so as to arrange the workpieces in respective housing seats so that the cutting wire intersects one workpiece at a time on at least three cutting planes, two cutting planes of which are orthogonal to each other.

According to an aspect of the invention, a robotic surgery system comprising at least one surgical instrument is provided.

The robotic surgery system can be a master-slave teleoperated system.

The robotic surgery system can be an automatic system.

### BRIEF DESCRIPTION OF THE DRAWINGS

Further features and advantages of the invention will appear from the following description of preferred embodiments, given as an indication and not as a limitation, with reference to the accompanying drawings (it should be noted that references to “an” embodiment as well as to “an” operating mode in this disclosure do not necessarily refer to the same embodiment or operating mode, and are to be understood as at least one, furthermore, for the purposes of conciseness and reduction of the total number of drawings, a certain drawing can be used to show the features of more than one embodiment as well as more than one operating mode, and not all elements of the drawing may be necessary for a certain embodiment/operating mode), in which:

FIG. 1 shows an axonometric view of a robotic surgery system, according to an embodiment:

FIG. 2 shows an axonometric view of a surgical instrument, according to an embodiment;

FIGS. 3A and 3B diagrammatically show an end-effector portion of a surgical instrument in two operating configurations, respectively, according to an embodiment, in which the actuation tendons are diagrammatically shown;

FIG. 4 shows an axonometric view of a portion of a surgical instrument comprising an end-effector at the distal end of the shaft, according to an embodiment, in which the actuation tendons are diagrammatically shown;

FIG. 5 shows an axonometric view of an end-effector of a surgical instrument, according to an embodiment, in which the actuation tendons are diagrammatically shown;

FIG. 6 shows an axonometric view of a portion of an end-effector of a surgical instrument, according to an embodiment;

FIG. 7 shows an axonometric view of the portion of the end-effector in FIG. 6 with separate parts;

FIG. 8A shows an axonometric view of a surgical instrument comprising an end-effector at the distal end of the shaft, according to an embodiment, in which the actuation tendons are diagrammatically shown;

FIG. 8B shows the end-effector and diagrammatically the actuation tendons in FIG. 8A:

FIG. 9 shows an axonometric view of a surgical instrument comprising an end-effector, according to an embodiment, in which the actuation tendons are diagrammatically shown;

FIG. 10 shows a plan view with separate parts of a portion of an end-effector of a surgical instrument, according to an embodiment;

FIG. 11 shows a plan view of the portion of the end-effector in FIG. 10 in an assembled and cutting configuration;

FIG. 12 shows an axonometric view of a portion of the end-effector in the cutting configuration shown in FIG. 11;

FIG. 13A shows a vertical elevation view of a blade link of the portion of the end-effector in FIG. 10;

FIG. 13B shows a vertical elevation view of a portion of the blade holder link of the end-effector portion in FIG. 10, according to an embodiment;

FIG. 14 is a diagram which diagrammatically shows in plan view the conformation assumed by a blade portion and a counter-blade portion in various mechanical cutting interference configurations, according to an embodiment;

FIGS. 15A and 15B are vertical elevation views of the end-effector portion in FIG. 11 according to the points of view indicated by arrows A andB, respectively;

FIG. 16 shows an axonometric view with separate parts of a portion of the end-effector in FIG. 11;

FIGS. 17A, 17B and 17C show a portion of the end-effector in FIG. 11 in a possible cutting sequence of a suture thread;

FIG. 18 shows a plan view with separate parts of a portion of an end-effector of a surgical instrument, according to an embodiment;

FIG. 19 shows a plan view with separate parts of an end-effector of a surgical instrument, according to an embodiment;

FIG. 20 shows the end-effector in FIG. 19 in an assembled and cutting configuration;

FIG. 21 shows an axonometric view of a portion of the end-effector in FIG. 19 in assembled configuration;

FIG. 22 shows a vertical elevation view of a counter-blade link of the end-effector in FIG. 19;

FIG. 23 shows a vertical elevation view of a portion of a counter-blade holder link of the end-effector in FIG. 19;

FIG. 24 shows an axonometric view with separate parts of a portion of the end-effector in FIG. 19;

FIG. 25 shows a vertical elevation view in assembled configuration of the portion of the end-effector in FIG. 24;

FIG. 26 is an electron microscope photographic image depicting a blade link and a counter-blade link placed on a face of a five euro cent coin;

FIG. 27A shows a vertical elevation view of a portion of an end-effector of a surgical instrument, according to an embodiment;

FIG. 28 shows a plan view in cutting configuration of a portion of an end-effector of a surgical instrument, according to an embodiment.

FIG. 29A shows a vertical elevation view of a portion of a first tip of an end-effector of a surgical instrument, according to an embodiment;

FIG. 29B is an enlargement of a detail of a blade link in FIG. 29A according to the point of view indicated by arrow B;

FIG. 29C shows an axonometry view of a detail of the portion of the first tip shown in FIG. 29A:

FIG. 30A shows a vertical elevation view of a blade link, according to an embodiment;

FIG. 30B shows a vertical elevation view of a counter-blade link, according to an embodiment;

FIG. 30C shows a vertical elevation view of a portion of an end-effector of a surgical instrument comprising the blade link in FIG. 30A and the counter-blade link in FIG. 30B in an assembled configuration;

FIG. 31 shows an axonometric view of a portion of a surgical instrument comprising an end-effector articulated at the distal end of the shaft, according to an embodiment, in which the actuation tendons are diagrammatically shown;

FIG. 32 shows an axonometric view of an end-effector of a surgical instrument according to an embodiment, in which the actuation tendons are diagrammatically shown;

FIG. 33 shows an axonometric view of a portion of the end-effector in FIG. 31;

FIGS. 34A and 34B show axonometric views with separate parts of the portion of the end-effector in FIG. 33 according to different points of view;

FIG. 35 shows a plan view with separate parts of a portion of an end-effector of a surgical instrument, according to an embodiment;

FIG. 36 is a diagram which diagrammatically shows in plan view the conformation assumed by a blade portion and a counter-blade portion of the end-effector in FIG. 35 in various mechanical cutting interference configurations, according to an embodiment;

FIG. 37 shows a plan view of a portion of the end-effector in FIG. 31 in a configuration in which the degree of freedom of opening/closing is partially closed and partially open, in which the actuation tendons are diagrammatically shown;

FIG. 38 shows a plan view of the portion of the end-effector in FIG. 37 in a configuration in which the degree of freedom of opening/closing is closed;

FIG. 39A shows an axonometric view of a portion of an end-effector of a surgical instrument in a configuration in which the degree of freedom of opening/closing is partially closed and partially open, according to an embodiment;

FIG. 39B shows the end-effector portion in FIG. 39A according to the point of view indicated by arrow B;

FIG. 39C shows an axonometric view of the portion of the end-effector in FIG. 39A according to a different point of view;

FIG. 39D shows the end-effector portion in FIG. 39C according to the point of view indicated by arrow D:

FIG. 40A shows a vertical elevation view of the portion of the end-effector in FIG. 39A in a configuration in which the degree of freedom of opening/closing is closed;

FIG. 40B shows the end-effector portion in FIG. 40A according to the point of view indicated by arrow B;

FIG. 40C shows a vertical elevation view of the portion of the end-effector in FIG. 40A according to a different point of view;

FIG. 40D shows the end-effector portion in FIG. 40C according to the point of view indicated by arrow D:

FIG. 41 shows a plan view of a second tip, according to an embodiment;

FIG. 42 shows a plan view of a blade holder link, according to an embodiment;

FIG. 43 shows a first tip, according to an embodiment;

FIG. 44 shows an axonometric view of a portion of the end-effector in FIG. 32;

FIG. 45 shows an axonometric view with separate parts of the portion of the end-effector in FIG. 44;

FIG. 46 shows a plan view with separate parts of an end-effector of a surgical instrument, according to an embodiment;

FIGS. 47A and 47B show axonometric views according to different points of view of a second tip of the end-effector in FIG. 46;

FIG. 48A shows a vertical elevation view of a portion of an end-effector, according to an embodiment, in an open configuration;

FIG. 48B shows the portion of the end-effector in FIG. 48A according to the point of view indicated by arrow B;

FIG. 48C is a diagram which diagrammatically shows in plan view the conformation assumed by a blade portion and a second tip of the end-effector in FIG. 48B in a mechanical cutting interference configuration;

FIG. 48D shows an axonometric view of the portion of the end-effector in FIG. 48A;

FIG. 49A shows a vertical elevation view of the portion of the end-effector in FIG. 48A in a partially closed and partially open configuration;

FIG. 49B shows the portion of the end-effector in FIG. 49A according to the point of view indicated by arrow B;

FIG. 49C is a diagram which diagrammatically shows in plan view the conformation assumed by a blade portion and a second tip of the end-effector in FIG. 49B in a mechanical cutting interference configuration;

FIG. 49D shows an axonometric view of the portion of the end-effector in FIG. 49A;

FIG. 50A shows a vertical elevation view of the portion of the end-effector in FIG. 48A in a partially closed configuration;

FIG. 50B shows the portion of the end-effector in FIG. 50A according to the point of view indicated by arrow B;

FIG. 50C is a diagram which diagrammatically shows in plan view the conformation assumed by a blade portion and a second tip of the end-effector in FIG. 50B in a mechanical cutting interference configuration;

FIG. 50D shows an axonometric view of a detail of the portion of the end-effector in FIG. 49A;

FIG. 51 shows a plan view with separate parts of an end-effector of a surgical instrument, according to an embodiment;

FIG. 52A shows an axonometric view of a surgical instrument comprising an end-effector at the distal end of the shaft, according to an embodiment, in which the actuation tendons are diagrammatically shown;

FIG. 52B shows the end-effector and diagrammatically the actuation tendons in FIG. 8A;

FIG. 53 shows an axonometric view of a surgical instrument comprising an end-effector, according to an embodiment, in which the actuation tendons are diagrammatically shown;

FIG. 54 is an electron microscope photographic image depicting an end-effector of a surgical instrument of the needle-driver/scissor gripper type at the distal end of a shaft, according to an embodiment;

FIG. 55 is an electron microscope photographic image depicting an end-effector of a surgical instrument of the scissor type at the distal end of a shaft, according to an embodiment;

FIG. 56 is an electron microscope photographic image depicting a blade link, according to an embodiment;

FIGS. 57A and 57B show plan views of a rotational joint, according to some embodiments;

FIGS. 57C and 57D show plan views of a rotational joint, according to an embodiment, in two opening configurations of the degree of freedom of opening/closing;

FIGS. 58A, 58B, 58C and 58D are block diagrams diagrammatically showing some possible steps of a manufacturing method, according to certain operating modes;

FIG. 59 diagrammatically shows a wire electro-erosion machine assembling a workpiece, according to a possible operating mode;

FIG. 60A shows a top plan view of a portion of a wire electro-erosion machine, according to a possible operating mode;

FIG. 60B shows a vertical elevation view of a fixture according to an embodiment;

FIG. 60C shows a housing portion of the fixture in FIG. 60B;

FIG. 61A shows an axonometric view of a sharpening step, according to a possible operating mode;

FIG. 61B shows a vertical elevation view of a jig assembling a workpiece at the end of a sharpening step, according to a possible operating mode;

FIG. 61C is a cross-section diagram of a workpiece diagrammatically showing a sharpening step, according to a possible operating mode;

FIG. 61D is a cross-sectional diagram of a workpiece at the end of a sharpening step, according to an embodiment;

FIG. 61E is a cross-section diagram of a workpiece diagrammatically showing a sharpening step, according to a possible operating mode;

FIG. 61F is a cross-sectional diagram of a workpiece at the end of a sharpening step, according to an embodiment:

FIG. 62A shows an axonometric view of a rotation step, according to a possible operating mode;

FIG. 62B shows a vertical elevation of a rotation step, according to a possible operating mode;

FIG. 63A shows an axonometric view of a shaping step, according to a possible operating mode;

FIG. 63B is an enlargement of the circled detail in FIG. 63A;

FIG. 63C shows a cross-section view of a workpiece subjected to sharpening and shaping, according to a possible operating mode;

FIG. 64 diagrammatically shows a step of curving, according to a possible operating mode;

FIG. 65 shows a plan view of a sharpening cutting path and a shaping cutting path, in accordance with a possible operating mode;

FIGS. 66A, 66B and 66C show a shaping cutting path, according to some possible operating modes;

FIG. 66D shows a semi-finished product comprising a plurality of shaped blades, according to an embodiment;

FIGS. 67A, 67B and 67C show a shaping cutting path, according to some possible operating modes;

FIG. 67D shows a semi-finished product comprising a plurality of shaped blades, according to an embodiment;

FIG. 68 is a photographic image showing a collection basket, according to an embodiment;

FIGS. 69A, 69B and 69C show a sequence of sharpening, rotating and shaping steps, according to a possible operating mode;

FIGS. 70A, 70B and 70C show a sequence of sharpening, rotating and shaping steps, according to some possible operating modes;

FIGS. 71, 72 and 73 show some possible steps of a method according to some possible operating modes, as well as some embodiments of a fixture;

FIGS. 74A, 74B and 74C show a sequence of sharpening, rotating and shaping steps, according to some possible operating modes;

FIG. 74D is a diagrammatic view according to the point of view indicated by arrow D in FIG. 74C;

FIG. 75 shows an axonometric view of a fixture in accordance with an embodiment which assembles a plurality of workpieces;

FIG. 76 diagrammatically shows in vertical elevation a possible step of a method, according to a possible operating mode;

FIGS. 77A, 77B and 77C diagrammatically show in vertical elevation some possible steps of a method, according to some possible operating modes.

### DETAILED DESCRIPTION OF SOME EMBODIMENTS

Reference throughout this description to “an embodiment” is meant to indicate that a particular feature, structure or function described in relation to the embodiment is included in at least one embodiment of the present invention. Therefore, the formulations “in an embodiment” in various parts of this description do not necessarily require that they all refer to the same embodiment. Furthermore, particular features, structures or functions such as those shown in different drawings can be combined in any suitable manner in one or more embodiments, unless expressly specified otherwise. Similarly, reference throughout this description to “an operating mode” is meant to indicate that a particular feature, structure or function described in connection with the operating mode is included in at least one operating mode of the present invention. Therefore, the formulation “in an operating mode” in various parts of this description does not necessarily all refer to the same operating mode. Furthermore, particular features, structures or functions such as those shown in different drawings can be combined in any suitable manner in one or more operating modes.

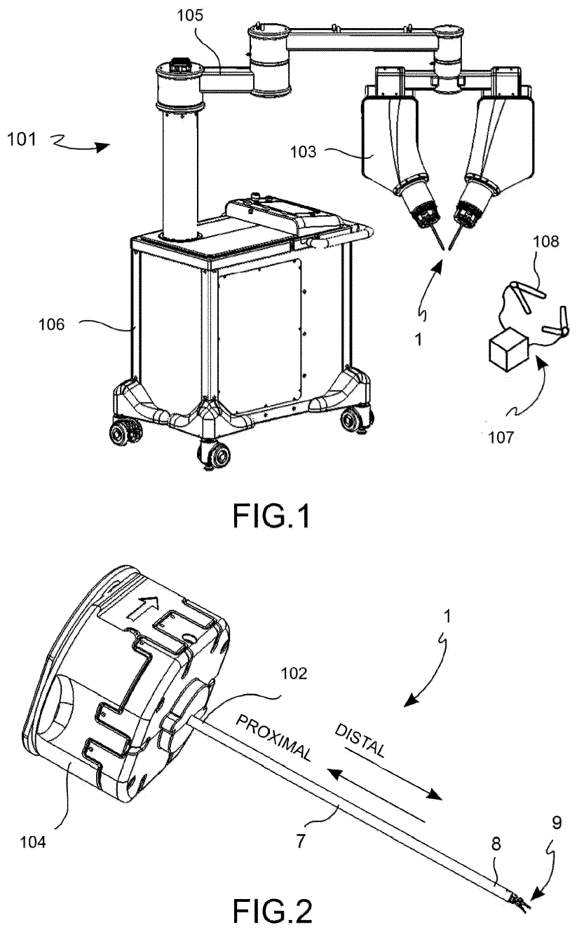

In accordance with a general embodiment, a surgical instrument 1 adapted to perform a cutting action is provided. Said surgical instrument 1 is particularly suitable, but not uniquely intended, for robotic surgery and can be connectable to a robotic manipulator 103 comprising motorized actuators of a robotic surgery system 101, as shown in FIG. 1, for example. For example, said surgical instrument 1 can be associated with a mechanical and manual control and actuation device.

The robotic surgery system 101 comprising said surgical instrument 1 is particularly suitable, but not uniquely intended, for robotic microsurgery operations. The robotic surgery system 101 can be intended for robotic laparoscopy operations.

Said surgical instrument 1 comprises an articulated end-effector 9, in other words an articulated end device 9. In accordance with an embodiment, said surgical instrument 1 comprises a shaft 7 or rod 7 and said articulated end-effector 9 at the distal end 8 of the shaft 7. Not necessarily said shaft 7 is a rigid shaft and for example can be a bendable shaft and/or an articulated shaft, although in accordance with a preferred embodiment said shaft 7 is a rigid shaft. A proximal interface portion 104 or backend portion 104 of the surgical instrument 1 can be provided at the proximal end 102 of the shaft 7, to form the interface with a robotic manipulator 103 of the robotic surgery system 101, as shown in FIG. 2, for example. A sterile barrier can be interposed between the robotic manipulator and the proximal interface portion 104 of the surgical instrument. For example, said proximal interface portion 104 can comprise a set of interface transmission elements for receiving the driving actions imparted by the robotic manipulator 103 and transmitting them to the articulated end-effector 9. In accordance with an embodiment, the surgical instrument 1 is detachably associated with the robotic manipulator 103 of the robotic surgery system 101.

The articulated end-effector 9 at the distal end 8 of the shaft 7 can comprise a plurality of links articulated to one another in one or more rotational joints movable by a number of pairs of antagonistic actuation tendons extending from the proximal interface portion 104 to the articulated end-effector 9 inside the shaft 7 ending in termination seats provided on at least some of the links of the articulated end-effector 9. The pair of actuation tendons of one or more pairs of antagonistic tendons can be obtained with a single tendon forming a round trip path from the proximal interface portion 104 of the instrument to a link of the articulated end-effector of the instrument.

Preferably, the term “link” refers to a body made in a single piece, i.e., a monobloc body.

Not necessarily all the links forming the articulated end-effector 9 are articulated, i.e., movable, with respect to one another and/or with respect to the distal end 8 of the shaft 7.

For example, said end-effector 9 can be an articulated cuff of the “roll-pitch-yaw” type according to a terminology widely adopted in the field. For example, said end-effector 9 can be an articulated end-effector 9 of the “snake” type, i.e., comprising a multitude of coplanar and/or non-planar rotational joints.

Said articulated end-effector 9 of the surgical instrument 1 comprises a support structure. The support structure can comprise prongs 3, 4 comprising a first prong 3 and a second prong 4 forming a support fork. Preferably, the support fork is made in a single piece, i.e., said two prongs 3, 4 are made in a single piece. In accordance with a preferred embodiment, said articulated end-effector 9 comprises a support link 2 comprising said support fork comprising said two prongs 3, 4.

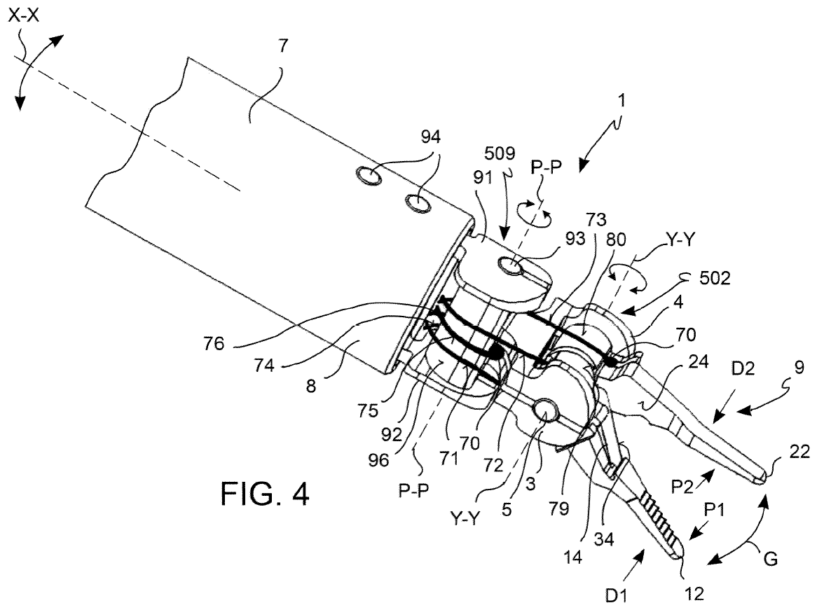

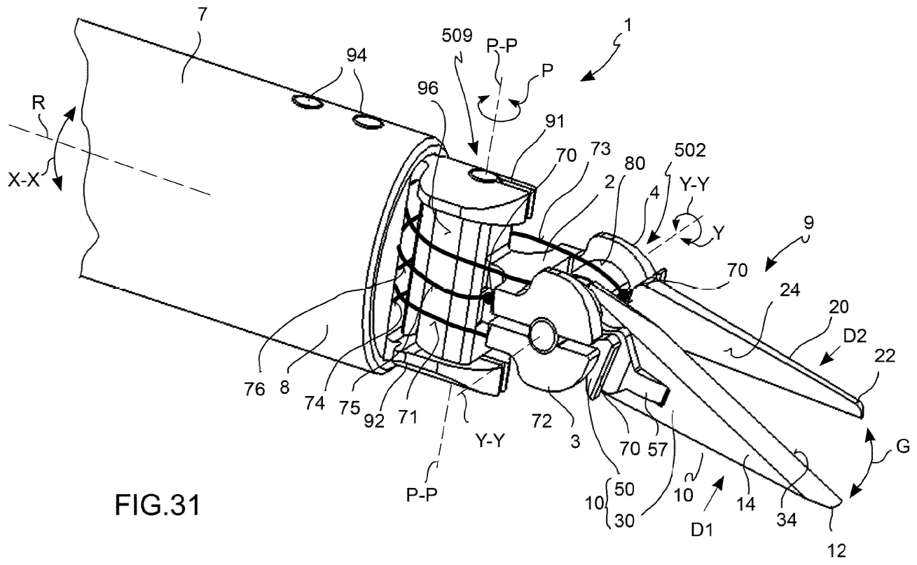

In accordance with an embodiment as shown in FIG. 4, for example, as well as in FIG. 31, for example, the support link 2 comprising the support fork with said prongs 3, 4 is a separate piece with respect to the shaft 7 and articulated thereto, by interposition between the support link 2 and the distal end 8 of the shaft 7 of a further connection link 90 rigidly fixed by means of a fixing device 94 (in the example shown as a pair of fixing pins 94, but alternatively the fixing device 94 can comprise plugs, rivets, staples, one or more threaded elements, interlocking profiles, or the like) at the distal end of the shaft 7 and in turn comprising two prongs 91, 92 articulated to the support link 2 with respect to the shaft 7 about a common proximal rotation axis P-P, or pitch axis P-P (the term “pitch” is used here arbitrarily and can indicate any orientation of the common rotation axis P-P). In such a case, therefore, the prongs 3 and 4 are articulated with respect to the distal end 8 of the shaft 7.

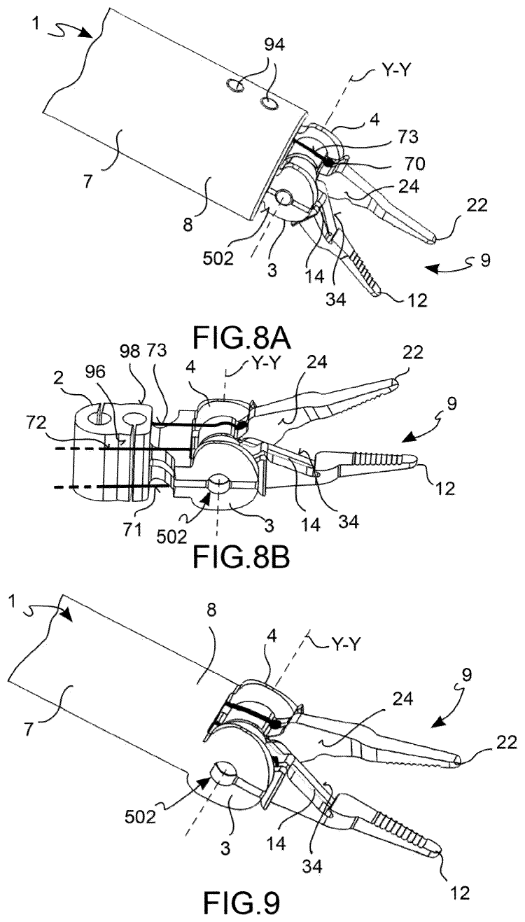

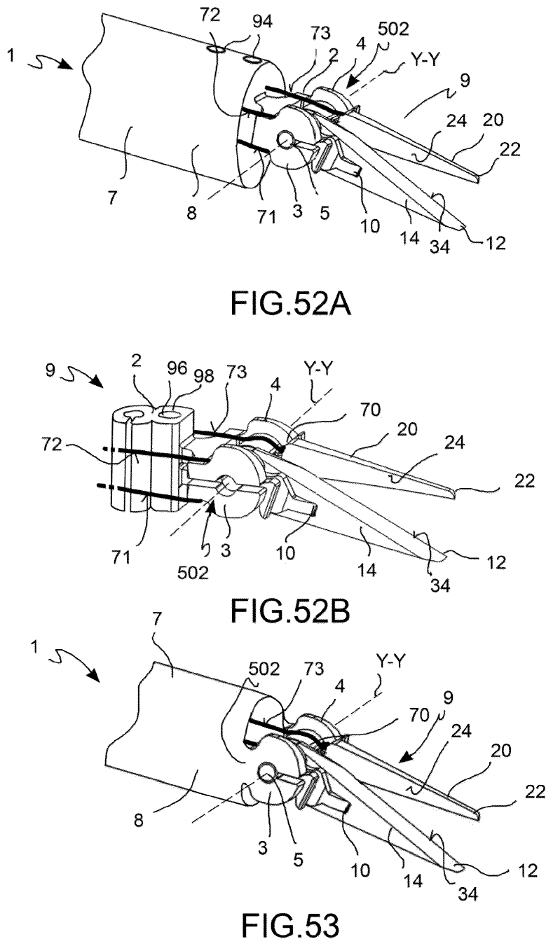

In accordance with an embodiment as shown in FIGS. 8A and 8B, for example, as well as in FIGS. 52A and 52B, for example, the support link 2 comprising the support fork with said prongs 3, 4 is a separate piece with respect to the shaft 7 and rigidly fixed thereto, i.e., not articulated, by means of a fixing device 94 (in the example shown as a pair of pins). In such a case, therefore, the prongs 3 and 4 are integral with the distal end 8 of the shaft 7.

In accordance with an embodiment as shown in FIG. 9, for example, as well as in FIG. 53, for example, the support structure or fork comprising said prongs 3, 4 is formed in a single piece with the distal end 8 of the shaft 7. In such a case, therefore, the prongs 3 and 4 are integral with respect to the distal end 8 of the shaft 7 and the articulated end-effector 9 further comprises the distal end 8 of the shaft 7 having the two prongs 3, 4, i.e., for the purposes of this disclosure and in these embodiments the distal end 8 of the shaft 7 comprising two prongs 3, 4 is understood as belonging to the articulated end-effector 9.

Said articulated end-effector 9 of the surgical instrument 1 comprises a first tip body 10, or first tip 10, comprising a first proximal attachment root 11 and a first distal free end 12. Not necessarily the body of the first tip 10 is made in a single piece, although in accordance with an embodiment the body of the first tip 10 is made in a single piece thereby forming a first tip link.

Said articulated end-effector 9 of the surgical instrument 1 further comprises a second tip body 20, or second tip 20, comprising a second proximal attachment root 21 and a second distal free end 22. Not necessarily the body of the second tip 20 is made in a single piece, although in accordance with an embodiment the body of the second tip 20 is made in a single piece thereby forming a second tip link.

Not necessarily the distal ends 12 and 21 of the first and second tips 10, 20 are free ends, and for example according to a variant at least one of said distal ends 12, 22 is guided or constrained for example by a hinge and/or a rail of a pantograph mechanism. In accordance with a preferred embodiment, the distal ends 12 and 21 of the first and second tips 10, 20 are the distal terminal free ends of the surgical instrument.

Preferably, said first and second tips 10 and 20 each have an elongated body, the elongated bodies of said first and second tips 10 and 20 being constrained to each other in respective proximal portions, or roots 11, 21, to rotate about a common rotation axis Y-Y being intended to form a terminal gripping device of the articulated end-effector 9 adapted to perform at least one cutting action. Therefore, the roots 11, 21 are adapted to form the rotational joint of the common rotation axis Y-Y and preferably lack elastic elements so as to avoid making seats for receiving an elastic deformation at the level of the roots, i.e., close to or at the articulation pin 5.

Said support structure, the first tip 10 and the second tip 20 are articulated together in a common rotation axis Y-Y defining an axial direction coincident with or parallel to the common rotation axis Y-Y.

Preferably, for clarity of presentation, an axial direction coincident or parallel with the direction of the common rotation axis Y-Y is defined. Preferably, for clarity of presentation, with reference to the first tip 10 an internal axial direction facing the second tip 20 along the axial direction is also defined and similarly said internal axial direction will be facing in the opposite direction with reference to the second tip 20, i.e., towards the first tip 10.

The proximal and distal directions (or senses) are understood as referring in accordance with the common meaning of the terms, as shown by the arrows in FIG. 2.

Preferably, for clarity of presentation, the term “radial” will refer to a direction which is substantially orthogonal to the common rotation axis Y-Y and incident thereto. Preferably, for clarity of presentation, it also means a longitudinal direction which globally can be substantially coincident with the longitudinal extension direction of the surgical instrument 1, as well as locally with the longitudinal extension direction of the elongated body of the first tip 10 and/or with the longitudinal extension direction of the elongated body of the second tip 20.

Said first root 11 of the first tip 10 and said second root 21 of the second tip 20 are axially next to each other.

Said first root 11 of the first tip 10 and said second root 21 of the second tip 20 are globally interposed between said prongs 3, 4 of the support structure. In other words, the assembly formed by said first root 11 of the first tip 10 and said second root 21 of the second tip 20 is interposed between said prongs 3, 4 of the support structure.

The first root 11 of the first tip 10 and the second root 21 of the second tip 20 are articulated with respect to the prongs 3, 4 of the support structure about said common rotation axis Y-Y defining a degree of freedom of orientation Y between the support structure and the assembly formed by said first tip 10 and said second tip 20. Therefore, the common rotation axis Y-Y (or a straight extension thereof) crosses said two prongs 3, 4, and said first and second roots 11, 12 and can be defined by an articulation pin 5. The support structure is preferably rigid, i.e., it is for example a rigid support fork, the relative position of the prongs 3, 4 is rigidly determined.

Furthermore, the first root 11 of the first tip 10 and the second root 21 of the second tip 20 are articulated with each other about said common rotation axis Y-Y, defining a relative degree of freedom of opening/closing G (or degree of freedom of cutting G, or degree of freedom of grip G according to a widely adopted terminology, although the activation of this degree of freedom does not necessarily result in a gripping action) between the first tip 10 and the second tip 20 to exert the cutting action. Thereby, the first free end 12 and the second free end 22 are relatively movable in an opening/closing direction, i.e., in a relative approaching/distancing direction.

Advantageously, said first tip 10 comprises a cutting edge 34 integral in rotation with the first free end 12 and said second tip 20 comprises a counter-blade portion 24 integral in rotation with the second free end 22. The counter-blade portion 24 preferably comprises a counter-blade surface 24 facing axially inwards.

A blade portion 14 of the body of the first tip 10 is axially elastically bendable and said counter-blade portion 24 of the second tip 20 is adapted to abut against said cutting edge 34 by axially elastically bending the body of said first tip 10. The blade portion 14 is preferably a portion of the body of the first tip 10 comprising said cutting edge 34 in a single piece, i.e., the cutting edge 34 belongs to the blade portion 14 of the body of the first tip 10.

Thereby, said cutting edge 34 of the first tip 10 and said counter-blade portion 24 of the second tip 20 reach a mechanical interference contact condition to exert a cutting action.

The mechanical interference contact between the cutting edge 34 and the counter-blade portion 24 resulting in the cutting action simultaneously deforms the blade portion 14 of the body of the first tip 10 in bending. The bending deformation of the blade portion 14 of the body of the first tip during the cutting action is preferably axially directed, i.e., it is directed substantially parallel to the common rotation axis Y-Y. 10

The deformed configuration of the blade portion 14 when the first tip 10 and the second tip 20 are in a substantially closed configuration is maximally bent, and in any case more bent than the configuration of the blade portion 14 when the first tip 10 and the second tip 20 are in a partially closed and partially open configuration. Preferably but not necessarily, when the opening angle is maximally open and the blade portion is free 14, the cutting edge 34 is straight and the blade portion 14 has a substantially planar configuration.

The at least one point of contact POC between the cutting edge 34 and the counter-blade portion 24 preferably varies in position and/or size as a function of the opening angle of the degree of freedom of opening/closing G and preferably tends to move in the distal direction as the opening angle is reduced, thereby accentuating the bending by elastic deformation of the body of the blade portion 14.

“Point of contact POC” preferably means the most distal portion of the contact area between cutting edge 34 and counter-blade portion 24, although the contact area can be similar to a point in some configurations of an embodiment.

The elastically deformable bending cutting edge 34 can be sharp, i.e., it can be subjected to sharpening in order to have a locally reduced thickness as compared to the thickness of the body of the blade portion 14 and/or a sharp conformation in the cross-section thereof. For example, the cross-section of the blade portion 14 has at the cutting edge 34 a pointed shape in which the faces of the blade link form an angle therebetween in the range of 30°-60°. Preferably, the cutting edge 34 of the first tip 10 is sharpened so as to be flush with an axially facing blade surface 35 of the blade portion 14 of the first tip 10 which is placed axially facing the counter-blade portion 24. In other words, the blade portion 14 of the body of the first tip 10 comprises a blade surface 35 facing axially inwards and said cutting edge 34 forming the edge of the blade surface 35.

During the cutting action, the blade surface 35 of the blade portion 14 can be in contact in at least one portion thereof with the counter-blade portion 24, exchanging frictional forces directed substantially in the opening/closing direction G.

In accordance with a preferred embodiment, said counter-blade portion 24 of the second tip 20 protrudes axially to bend the first tip 10. The inclusion of such a protruding counter-blade portion 24 allows it to abut against the cutting edge 34 of the first tip 10, bending the body of the first tip 10.

In accordance with an embodiment, the protrusion of the counter-blade portion 24 is accentuated in a distal direction along the longitudinal extension of the body of the second tip 20.

In accordance with an embodiment, said counter-blade portion 24 comprises a curved protruding surface having a concavity facing axially inwards.

In accordance with an embodiment, the counter-blade portion 24 of the second tip 20 protrudes towards the rotational approaching footprint of the blade portion 14 of the first tip 10, to elastically bend the blade portion 14 when the counter-blade portion 24 is in mechanical interference contact with the cutting edge 34. In other words, the counter-blade portion 24 protrudes axially inwards. In accordance with an embodiment, said protruding of the counter-blade portion 24 accentuates towards the distal direction, i.e., away from the common rotation axis Y-Y along the longitudinal extension of the second tip 20 and preferably said protruding is maximum close to or at the distal end 32 of the blade portion 14 of the first tip 10.

Preferably, the term “rotational approaching footprint” is meant to indicate the volume of space which can be occupied by the body of an element during the relative rotation movement of the closing of the degree of freedom of grip G.

Not necessarily the blade portion 14 and thus the blade surface 35 of the first tip 10 is a planar portion i.e., lying on a plane and can be a curved or arched portion, although in accordance with an embodiment the blade portion 14 is a planar portion.

In accordance with an embodiment, the body of the blade portion 14 has a main two-dimensional extension, i.e., lying on a preferably flat or arched lying surface, and has a substantially reduced thickness with respect to the extension on said preferably flat or arched lying surface.

In accordance with an embodiment, the cutting edge 34 of the blade portion 14 is substantially straight in the preferably flat or arched lying surface, avoiding concavity in the lying surface of the body of the blade portion 14.

Preferably, the thickness of the blade portion 14 is significantly less than the thickness of the first root 11 of the first tip 10 and the second root 21 of the second tip 20, and is chosen so that the blade portion 14 is elastically bendable when in operating conditions, transversely to the longitudinal extension of the cutting edge 34, and in particular in the direction of the thickness of the blade portion 14. In particular, the blade portion 14 is preferably more bendable than the body of the second tip 20, and preferably also more bendable than the body of the counter-blade portion 24. The bendability of the blade portion 14 and thus the bendability of the cutting edge 34 is understood in the direction of the thickness thereof, i.e., in a direction orthogonal to the lying surface, whether flat or arched, of the blade portion 14. For example, the blade portion 14 has an arched, i.e., concave, conformation having a concavity facing in a direction exiting from/entering into the lying plane and in such a case the lying surface of the body of the blade portion 14 is an arched surface as is the blade surface 35.

Not necessarily the blade portion 14 and thus the cutting edge 34 must be elastically deformable in the lying surface, i.e., a bendability is not necessarily included in a direction orthogonal to the thickness thereof.

The ratio between the thickness of the body of the blade portion 14 at the level of the blade surface 35 (excluding in this evaluation the thickness of the cutting edge 34, which as mentioned is preferably sharpened) and the thickness of the first root 11 of the first tip 10 and/or the thickness of the second root 21 of the second tip 20 can be between ⅕ and 1/20. In absolute value the thickness of the blade portion 14 can be between 0.1 mm and 0.5 mm and in accordance with an embodiment substantially equal to 0.2 mm.

As mentioned above, the blade portion 14 is integral in rotation with the first tip 10. Thereby, the cutting edge 34 is integral in rotation with the first free end 12 and, being elastically bendable, the cutting edge 34 can be elastically deformed with respect to the first tip 10 integral therewith in rotation when in operating conditions. The elastic deformation of the cutting edge 34 preferably occurs in a transverse direction with respect to the longitudinal extension direction of the elongated body of the first tip 10, i.e., in a transverse direction with respect to the direction joining the first proximal attachment root 11 and the first distal free end 12 of the first tip 10, in other words in the direction of the thickness of the blade portion 14.

In accordance with an embodiment, the blade portion 14 is substantially planar when in a non-deformed configuration, i.e., it lies on a definable lying plane. The bending elasticity of the blade portion 14 tends to bring the blade portion 14 back into said non-deformed planar configuration. Therefore, the blade surface 35 facing axially inwards can be parallel, and preferably also aligned for example seamlessly, to an axially facing internal contact surface 83 of the first root 11 of the first tip 10. Preferably, the cutting edge 34 is straight when in a non-deformed condition i.e., extends substantially in a straight line parallel to, and preferably as a straight extension of, the axially facing internal contact surface 83 of the first root 11 of the first tip 10. In other words, in accordance with an embodiment, the cutting edge 34 extends parallel to the definable lying plane of the blade portion 14.

The cutting edge 34 of the blade portion 14 can be aligned with the longitudinal extension direction X-X of the shaft 7 or rod 7 in at least one operating configuration, for example in the case in which the shaft 7 is a straight and rigid shaft and the cutting edge 34 is out of contact with a protruding portion of the counter-blade portion 24.

Preferably, for clarity of presentation, a first back side D1 of the first tip 10 and a second back side D2 of the second tip 20 are defined with reference to the relative degree of freedom of opening/closing G, said first back side D1 and second back side D2 opposingly face each other, and a first cutting side P1 of the first tip 10, in which said cutting edge 34 belongs to the first cutting side P1 of the first tip 10, and a second cutting side P2 of the second tip 20 opposite to and substantially facing in rotation the first cutting side P1 are defined, although preferably they are mainly next to each other and can be in contact in at least said cutting edge 34 and said counter-blade portion 24 when the degree of freedom of opening/closing G is in a closing configuration or at least partially closed, exerting the cutting action.

In accordance with an embodiment, said counter-blade portion 24 can be made sloping in a direction which is transverse, preferably orthogonal, to the longitudinal extension of the body of the second tip 20 and is also transverse, preferably orthogonal, to the common rotation axis Y-Y, i.e., in other words, said counter-blade portion 24 can be made sloping in the direction which joins the back side D2 with the gripping side of the second tip 20, preferably protruding more towards the back side D2. The counter-blade portion 24 is not necessarily made sloping, even while protruding.

In accordance with an embodiment, said counter-blade portion 24 is a curved surface. Thereby, the counter-blade portion 24 protrudes due to the arched shape thereof. The concavity of the counter-blade portion 24 is preferably axially and internally facing i.e., in a direction parallel to the common rotation axis Y-Y and facing the rotational footprint of the blade portion 14.

The counter-blade portion 24 can act as a wedge to appropriately bend the cutting edge 34 and the blade portion 14 to exert the cutting action substantially along the entire longitudinal extension of the counter-blade portion 24.

As mentioned above, the first tip 10 can be made in a single piece forming a first tip link, or alternatively the first tip 10 can be formed from separate pieces, i.e., from separate links integral with one another in rotation.

In accordance with a preferred embodiment, the first tip 10 is formed by two links comprising a blade link 30 and a blade holder link 50 integral with each other in rotation, in which the blade link 30 is made in a single piece and the blade holder link 50 is made in a single piece. The provision of the first tip 10 formed by only two links 30, 50 integral in rotation still allows keeping the number of parts to be assembled small and at the same time allows modulating the mechanical properties as well as the production parameters of the individual links 30, 50. Therefore, the blade link body 30 of the first tip 10 comprises in a single piece said blade portion 14 with said cutting edge 34 and a blade link root 31, and the blade holder link body 50 of the first tip 10 comprises in a single piece a blade holder link root 51, in which the blade link root 31 and the blade holder link root 51 are next to and in direct and intimate contact with each other, forming jointly said first root 11 of the first tip 10. Therefore, in this case, said degree of freedom of orientation of yaw Y about the common rotation axis Y-Y will be between the support structure and the assembly formed by said blade link 30 and said blade holder link 50 of the first tip 10 and said second tip 20 and said relative degree of freedom of opening/closing G about the common rotation axis Y-Y, the cutting action will be exerted between the second tip 20 and the assembly formed by: said blade link 30 and said blade holder link 50.

By virtue of such a pack arrangement of the roots, impingements of the root 31 of the blade link 30, which is preferably thinner, with respect to the articulation pin 5 are avoided so as to provide a satisfactory certainty of positioning the cutting edge 34 with respect to the counter-blade portion 24 for each opening angle of the degree of freedom of opening/closing G, thus providing extreme cutting precision.

In accordance with a preferred embodiment, the root 31 of the blade link 30 is interposed between the root 51 of the blade holder link 50 and the second root 21 of the second tip 20. Alternatively, in accordance with an embodiment, the blade holder link root 51 is interposed between the blade link root 31 of the first tip 10 and the second root 21 of the second tip 20. i.e., the blade link root 31 is interposed between the first prong 3 of the support structure and the root 51 of the blade holder link 50 of the first tip 1. Thereby, the blade portion 14 is also interposed between the body of the blade holder link 50 and the body of the second tip 20.

The roots preferably have a cylindrical geometry about the common rotation axis Y-Y, and where the root 31 of the blade link 30 has a substantially smaller thickness than the root 51 of the blade holder link 50 and the second root 21, the root 31 of the blade link 30 thus has a discoidal-type cylindrical geometry, in which the cylinder bases of the cylindrical geometry of each root are formed by the respective axially facing contact surfaces. Therefore, the roots are substantially stacked in axis on the common rotation axis Y-Y and preferably each comprise a through hole which receives the articulation pin 5. Each root is preferably a rigid body designed to define the rotational joint of the common rotation axis Y-Y (for example adapted to receive the articulation pin 5) and where the root 31 of the blade link 30 is an elastic root, it is preferably made flat and is interposed in a pack between the prongs, for example between the root 51 of the blade holder link 50 and the second root 21 of the second tip 20, preventing it from exerting an axial elastic preload action in the area of the articulation pin 5. The elastic action of the blade link 30 is preferably located only in the blade portion 14.

By virtue of such roots next to one another, it is possible to keep the proximal dimension of the rotational joint defining said common rotation axis Y-Y compact and to avoid providing elastic elements which exert an axial preload between the roots as well as between the roots and the prongs.

Preferably, the body of the blade link 30 is also longitudinally elongated and comprises a blade link end which does not necessarily coincide with the first free end 12 of the first tip 10.

The material of the blade link 30 can be a different material than the material of the blade holder link 50. For example, the counter-blade link 40 and the support link 2, where present, can be made of a single metal material, such as steel.

In accordance with an embodiment, said blade link 30 of the first tip 10 is made by shaping, i.e., by cutting, suitably a substantially flat elastic sheet or strip. For example, the elastic sheet or strip can be made of spring steel and shaped by wire electro-erosion (WEDM) and/or photo-etching and/or laser cutting and/or chemical etching. Preferably, the elastic sheet or strip is sharpened on one edge thereof to form the cutting edge 34 of the blade link 30.

The sharpening can be carried out by wire electro-erosion (WEDM) and/or grinding, for example stone or diamond grinding. In accordance with an embodiment, first the elastic sheet or strip is shaped by wire electro-erosion (WEDM) in a step in which the cutting edge flows in a direction substantially orthogonal to the lying plane of the sheet or strip, then one or more edges of the shaped sheet or strip are sharpened by wire electro-erosion (WEDM) in a step in which the cutting edge flows in a direction not orthogonal to the lying plane of the shaped sheet or strip.

In accordance with an embodiment, the body of the blade link 30 has a two-dimensional main extension, i.e., lying on a preferably flat or arched lying surface, and has a substantially reduced thickness with respect to the extension on said preferably flat or arched lying surface. The thickness of the blade link 30 is preferably constant, except for the cutting edge 34 which, as mentioned, can have a reduced thickness in order to be sharpened.

In accordance with an embodiment, the cutting edge 34 of the blade link 30 is substantially straight in the preferably flat or arched lying surface, avoiding the provision of concavities in the lying surface of the body of the blade link 30.

Preferably, the thickness of the blade link 30 is significantly smaller than the thickness of the root 51 of the blade holder link 50 and is chosen so that the blade portion 14 is elastically bendable, when in operating conditions, transversely to the longitudinal extension of the blade link 30, i.e., in the direction of the thickness thereof. In particular, the blade link 30 may be more bendable than the counter-blade portion 24. Such a lying surface of the body of the blade link 30 can substantially correspond to the lying plane of the starting metal strip or sheet which suitably processed forms the blade link 30, even though in accordance with a possible embodiment the body of the blade link 30 is forced to have an arched, i.e., concave, conformation having a concavity facing in a direction exiting from/entering the lying plane of the starting elastic strip or sheet and in this case the lying surface of the blade link body will be an arched surface.

The material of the blade link 30 can be a different material than the material of the blade holder link 50. For example, the blade link 30 can be made of spring steel. For example, the blade link 30 can be made of spring steel.

The ratio of the thickness of the root 31 of the blade link 30 to the thickness of the root 51 of the blade holder link 50 and/or the thickness of the second root 21 of the second tip 20 can be between ⅕ and 1/20. In absolute value the thickness of the root 31 of the blade link 30 can be between 0.1 mm and 0.5 mm and in accordance with an embodiment substantially equal to 0.2 mm.

Where said support structure having the prongs 3, 4 (support structure for example formed by the support link 2 or by the distal end 8 of the shaft), the blade link 30 and the blade holder link 50 of the first tip 10 and the second tip 20 are made in mutually separate pieces and said blade link 30 is integral in rotation with the blade holder link 50, the cutting edge 34 as well as the blade portion 14, being elastically bendable, can bend elastically with respect to the blade holder link 50 when in operating conditions.

In accordance with an embodiment, the blade link 30 and the blade holder link 50 further comprise respective drag engagement portions 37, 57 to make the blade link 30 and the blade holder link 50 integral in rotation. The drag engagement can be obtained by an engagement between the blade link 30 and the blade holder link 50.

The drag engagement between the blade link 30 and the blade holder link 50 can be arranged distally with respect to the common rotation axis Y-Y. In such a case, the drag engagement portion 37 (or drag portion 37) of the blade link 30 is preferably positioned far from the blade link root 31 so as to ensure a precise drag, even though the drag portion 37 of the blade link 30 can be positioned at the blade link root 31 to achieve a more advantageous mechanical transfer. In accordance with a preferred embodiment, the drag engagement between the blade link 30 and the blade holder link 50 is placed distally with respect to the first root 11 of the first tip 10.

In accordance with an embodiment in which the first tip 10 is in a single piece i.e., it is a first tip link and the second tip 20 is in a single piece i.e., it is a second tip link, the articulated end-effector 9 is formed by three separate pieces comprising: the support structure (formed by the support link 2 or by the distal end 8 of the shaft 7), the first tip 10 and the second tip 20 articulated to each other in a common rotation axis Y-Y, i.e., constrained to rotate with respect to a common rotation axis Y-Y, or common yaw rotation axis Y-Y (the term “yaw” is arbitrarily used here and can indicate any orientation of the common rotation axis Y-Y, although in accordance with a preferred embodiment it is meant to indicate a common yaw rotation axis Y-Y non-parallel and preferably orthogonal to the common proximal pitch rotation axis P-P already mentioned). In other words, in accordance with this embodiment, the articulated end-effector 9 consists of exactly said three pieces mutually articulated in said common axis Y-Y and suitably movable by actuation tendons plus a further piece which is an articulation pin 5 defining said common axis Y-Y (in total four pieces, the actuation tendons are excluded from the count).

In accordance with an embodiment in which the first tip 10 is formed i.e., consists of said blade link 30 and said blade holder link 50 and the second tip 20 is in single piece i.e., is a second tip link, the articulated end-effector 9 is formed by four separate pieces comprising: the support structure (formed by the support link 2 or by the distal end 8 of the shaft 7), the blade link 30 and the blade holder link 50 of the first tip 10 integral with each other in rotation, and the second tip 20 mutually articulated in a common rotation axis Y-Y. In other words, in accordance with this embodiment, the articulated end-effector 9 consists of exactly said four pieces articulated together in said common axis Y-Y and suitably movable by actuation tendons plus a further piece which is an articulation pin 5 defining said common axis Y-Y (in total five pieces, the actuation tendons are excluded from the count).

In accordance with an embodiment in which the first tip 10 is in a single piece i.e., it is a first tip link and the second tip 20 is in a single piece i.e., it is a second tip link, the articulated end-effector 9 is formed by four links which are: the support link 2, the first tip 10 and the second tip 20 articulated to each other in the common distal rotation axis Y-Y by means of said articulation pin 5, and a link 90 to the shaft 7 articulated proximally to the support link 2 in the common proximal rotation axis P-P by means of a further proximal articulation pin 93. In other words, in accordance with this embodiment, the articulated end-effector 9 consists of exactly said four links 2, 10, 20, 90 plus two further pieces which are the articulation pin 5 defining said common distal rotation axis Y-Y and the proximal articulation pin 93 defining said common proximal rotation axis P-P, (in total six pieces, the actuation tendons are excluded from the count). By virtue of this embodiment, and where said common distal yaw rotation axis Y-Y and said common proximal pitch rotation axis P-P are non-2,5 parallel to each other, and preferably orthogonal, an articulated end-effector 9 of the articulated pitch-yaw-grip cuff type, i.e., pitch-yaw-cut (P,Y,G) is allowed. By virtue of this embodiment, and where the connection link 90 is made in a single piece with the distal end 8 of the shaft 7 (not shown in the figure), the articulated end-effector 9 will still be formed by said six pieces which are: the distal end 8 of the shaft 7, the support link 2, the first tip 10, i.e., the first tip link, the second tip 20, i.e., the second tip link, and said two articulation pins 5, 93, 30

In accordance with an embodiment in which the first tip 10 is formed i.e., consists of said blade link 30 and said blade holder link 50 and the second tip 20 is in single piece i.e., is a second tip link, the articulated end-effector 9 is formed by five links which are: the support link 2, the blade link 30, the blade holder link 50 and the second tip 20 articulated together in the common distal rotation axis Y-Y by means of said articulation pin 5, and a link 90 to the shaft 7 articulated proximally to the support link 2 in the common proximal rotation axis P-P by means of a further proximal articulation pin 93. In other words, in accordance with this embodiment, the articulated end-effector 9 consists of exactly said five links 2, 20, 30, 50 90 plus two further pieces which are the articulation pin 5 defining said common distal rotation axis Y-Y and the proximal articulation pin 93 defining said common proximal rotation axis P-P, (in total seven pieces, the actuation tendons are excluded from the count). Where said common distal yaw rotation axis Y-Y and said common proximal pitch rotation axis P-P are non-parallel to each other, and preferably orthogonal, an articulated end-effector 9 of the articulated pitch-yaw-grip cuff type, i.e., pitch-yaw-cut (P,Y,G) is allowed. Where the connection link 90 is made in a single piece with the distal end 8 of the shaft 7 (not shown in the figure), the articulated end-effector 9 will still be formed by said seven pieces which are: the distal end 8 of the shaft 7, the support link 2, the blade link 30 and the blade holder link 50 of the first tip 10, the second tip 20. i.e., the second tip link, and said two articulation pins 5, 93.

Those skilled in the art will appreciate that minimizing the number of pieces greatly simplifies the assembly of the articulated end-effector 9 of the surgical instrument 1, making it suitable for an extreme miniaturization. In particular, avoiding the provision of elastic preload elements in the axial direction (such as Belleville-type elastic washers fitted on the articulation pin 5), i.e., in the direction of the common rotation axis Y-Y between the prongs 3, 4 of the support structure, it is possible to simplify the assembly of the pieces and therefore an extreme miniaturization of the articulated end-effector 9 is favored, as well as consequently of the cross-section of the shaft 7, while ensuring a satisfactory strength and resistance to the stresses which can arise when in operating conditions.

A degree of freedom of roll R integral with the shaft 7 and preferably also with the backend portion 104 can be provided, for example a degree of freedom of roll R which allows the entire surgical instrument 1 to be rotated about the longitudinal extension axis X-X of the shaft 7.

In accordance with a preferred embodiment, the first root 11 of the first tip 10 is in direct and intimate contact with the first prong 3 of the support structure and the second root 21 of the second tip 10 is in direct and intimate contact with the second prong 4 of the support structure. Thereby, the assembly formed by said first root 11 and said second root 12 is interposed between the prongs 3, 4 and in intimate and direct contact therewith. Therefore, the provision of Belleville-type spring washers between the prongs of the support structure and the roots of the tips is avoided. Such a configuration allows minimizing the axial footprint of the roots of the tips and of the prongs of the support structure and allows simplifying the assembly as it avoids the need to assemble the pieces by counteracting the elastic reaction to the rotation axis Y-Y which would be given by such Belleville-type spring washers.

In accordance with a preferred embodiment, said first root 11 of the first tip 10 comprises a first axially facing external contact surface 81, and said first prong 3 comprises a first axially facing internal contact counter-surface 87, in which said second root 21 of the second tip 20 comprises a second axially facing external contact surface 82, and said second prong 4 comprises a second axially facing internal contact counter-surface 88. Preferably, said first external contact surface 81 of the first root 11, said first internal contact counter-surface 87 of the first prong 3, said second external contact surface 82 of the second root 21 and said second internal contact counter-surface 88 of the second prong 4 are all parallel to one another, and preferably each of them extends in a plane substantially orthogonal to the common rotation axis Y-Y.

In accordance with an embodiment, the first root 11 of the first tip 10 and the second root 21 of the second tip 20 are in direct and intimate contact. Therefore, the first root 11 of the first tip 10 further comprises a first axially facing internal contact surface 83 and the second root 21 of the second tip 20 comprises a second axially facing internal contact surface 84, said first internal contact surface 83 of the first tip 10 is in direct and intimate contact with said second internal contact surface 84 of the second tip 20. It is thus possible to obtain a package arrangement of the roots between the prongs of the support structure. By virtue of such a pack arrangement of the roots, an axial reaction is provided to the elastic bending of the body of the first tip during the cutting action.

In accordance with an embodiment, said first internal contact surface 83 of the first tip 10 is parallel to said second internal contact surface 84 of the second tip 20. Preferably, all said contact surfaces are parallel to one another and even more preferably each extend in a plane orthogonal to the common rotation axis Y-Y; i.e., in other words, said first external contact surface 81 and said first internal contact surface 83 of the first tip 10, said second external contact surface 82 and said second internal contact surface 84 of the second tip 20, said first internal contact counter-surface 87 of the first prong 3 and said second internal contact counter-surface 88 of the second prong 4 preferably are all parallel to one another and even more preferably each extend in a plane orthogonal to the common rotation axis Y-Y.

Where the first root 11 of the first tip 10 is formed by a root 31 of a blade link 30 and a root 51 of a blade holder link 50 in direct and intimate contact therebetween, said first external contact surface 81 and said opposite first internal contact surface 83 of the first root 11 of the first tip 10 will belong to different links of the articulated end-effector 9, i.e., one between said first external contact surface 81 and said first internal contact surface 83 will belong to the blade link root 31 while the other will belong to the blade holder link root 51. In accordance with a preferred embodiment in which the blade link root 31 is interposed between the blade holder link root 51 and the second root 21 of the second tip 20, said first external contact surface 81 will belong to the root of blade holder link 51 and said opposite first internal contact surface 83 will belong to the blade link root 31. Furthermore, where the first root 11 of the first tip 10 is formed by a blade link root 31 and a blade holder link root 51 in direct and intimate contact therebetween, two further opposite contact surfaces 85, 86 will be provided in direct and intimate contact therebetween, in which a first further contact surface 85 will belong to the blade link root 31 and a second further contact surface 86 will belong to the blade holder link root 51. Preferably, said two further opposite contact surfaces 85, 86, in direct and intimate mutual contact, of the blade link root 31 and the blade holder link root 51, respectively, are both parallel to the other contact surfaces of the first root 11 of the first tip 10 and the second root 21 of the second tip 20.

Although the manufacture of the pieces by means of a wire electro-erosion process allows obtaining boosted tolerances, minimum local micro-clearances can be provided in the direction of the common rotation axis Y-Y of the order of a fraction of a tenth of a millimeter between at least some of said contact surfaces of the roots and/or the prongs to ensure a direct and intimate contact and at the same time allow the relative rotation about the common rotation axis Y-Y during the actuation of the degree of freedom of opening/closing G and/or yaw Y. The articulation pin 5 can be in interference, i.e., integral in rotation with at least one of the roots and/or the prongs.

In particular, as a consequence of the fact that the support structure with two prongs 3, 4, the first root 11 of the first tip 10 and the second root 21 of the second tip 20 are made in at least three separate pieces, an albeit minimal micro-clearance in the axial direction, i.e., in the direction of the common rotation axis Y-Y between the respective contact surfaces, is however necessary. Therefore, the wording “direct and intimate contact” also intends to indicate the embodiments in which a minimum micro-clearance is in any case provided between at least some of, but also all, the counter-contact surfaces of the prongs of the support structure and the contact surfaces of the roots.

By virtue of the fact that the support structure with two prongs 3, 4, the first root 11 and the second root 21 are made in at least three separate pieces, imposing a minimum clearance in the direction of the common rotation axis Y-Y as explained above, the degree of freedom of opening/closing G can be maneuvered in rotation in a precise and controlled manner both in the opening and in the closing direction, in order to exert the cutting action.

Where the first tip 10 is formed by two links 30, 50, during the cutting action and in particular for relatively high opening angles of the degree of freedom of opening/closing G (e.g., angle greater than)25°, the mechanical interference contact between the cutting edge 34 of the blade link 30 and the counter-blade portion 24, therefore, can generate a minimum micro-displacement of the order of a hundredth of a millimeter of the blade link root 31 along the articulation pin 5. For example, in accordance with an embodiment the thickness of the blade link root 31 is about 0.2 mm and the overall micro-clearance in the direction of the common rotation axis Y-Y which is in operating conditions distributed locally between the contact surfaces of the blades and the roots is globally about 0.02 mm, and when in operating conditions the local micro-clearance in the direction of the common rotation axis Y-Y between the blade link root 31 of the first tip 10 and the second root 21 of the second tip 20 is about 0.01 mm, i.e., substantially equal to 1/20 of the thickness of the blade link root 31.

In accordance with a preferred embodiment, said first root 11 of the first tip 10 comprises a first through hole 16 and said second root 21 of the second tip 20 comprises a second through hole 26, said first through hole 16 and said second through hole 26 are aligned in axis with said common rotation axis Y-Y. In accordance with an embodiment, an articulation pin 5 is received inside said first and second through holes 16, 26.

In accordance with an embodiment, said first through hole 16 of the first root 11 and said second through hole 26 of the second root 21 are all circular through holes coaxial to said common rotation axis Y-Y and receive a single articulation pin 5 extending in the direction of the common rotation axis Y-Y from a first prong 3 of the support structure to a second prong 4 of the support structure. In accordance with an embodiment, said first through hole 16 of the first root 11 and said second through hole 26 of the second root 21 all have substantially the same diameter and receive said articulation pin 5 in direct and intimate contact for the entire circumferential extension of the respective hole edge. Thereby, it is possible to exert a reaction to the cutting action exerted by the cutting edge 34. In particular, during the cutting action the opening angle of the degree of freedom of opening/closing G is progressively reduced, thus resulting in a mechanical interference contact between the cutting edge 34 (and preferably also of the blade surface 35 as mentioned above) and the counter-blade portion 24, and therefore a direct friction force in the opening direction is generated on the cutting edge 34 (and preferably also on the blade surface 35) axially facing the blade portion 14 which is in contact with the counter-blade surface of the counter-blade portion 24 which is balanced by a reaction to the friction of the cutting action exchanged in a portion of mutual contact between the hole edges of the roots 11, 21 and the articulation pin 5. The friction reaction of the cutting action is preferably directed substantially along a radial direction with respect to the common rotation axis Y-Y. The friction reaction of the cutting action preferably affects an arc surface of the thickness of the hole edge of the first root 11 and/or of the second root 21.

Where the first root 11 of the first tip 10 is formed by a root 31 of a blade link 30 and a root 51 of a blade holder link 50 in direct and intimate contact with each other, each of said blade link root 31 and blade holder link root 51 will be provided with a first through hole 16, according to any one of the embodiments described above. In such a case, the first through hole 16 of the root 51 of the blade holder link 50 and the first through hole 16 of the root 31 of the blade link 30 can be coaxial circular holes and can have the same diameter. In such a case, the hole edge 36 of the through hole 16 of the root 31 of the blade link 30 of the first tip 10 can comprise an arc surface 38 in direct and intimate contact with the articulation pin 5 to exert said reaction to the friction force generated by the cutting action.

Where at least some, but also all, of the through holes of the roots are made by wire electro-erosion (WEDM), a radial cutting channel 19, 29, 39 is provided on the respective root between the hole edge and the external edge of the respective root as an effect of the continuous cutting path of the cutting wire used for making the through holes by wire electro-erosion. Preferably, the arrangement of the radial cutting channel on the respective root is studied based on the static or dynamic behavior when in operating conditions. In particular, in accordance with a preferred embodiment, the cutting channel 39 of the root 31 of the blade link 30 is radially offset with respect to the cutting channel 29 of the second root 21 of the second tip 20 to prevent the edges of the cutting channels 29, 39 from interlocking with each other during the opening/closing action.

In accordance with an embodiment, the through hole of the prong of each of said two prongs 3, 4 is a circular through hole coaxial to said common rotation axis Y-Y. Where the prongs 3, 4 of the support structure are made by wire electro-erosion, at least one radial channel between the hole edge and the external edge of the respective prong can be provided on the prong.

In order to move the articulated end-effector 9 about said common axes of proximal and/or distal rotation i.e., pitch P-P and/or yaw Y-Y to activate the degrees of freedom of the articulated end-effector 9, preferably the surgical instrument 1 comprises a plurality of pairs of antagonistic actuation tendons extending from the backend portion to the articulated end-effector 9 through the shaft 7 and ending at the articulated end-effector 9, as explained below.

In accordance with a preferred embodiment, the first tip 10 comprises a first termination seat 15 which receives a first pair of antagonistic tendons 71, 72, and the second tip 20 comprises a second termination seat 25 which receives a second pair of antagonistic tendons 73, 74. Those skilled in the art will appreciate that in this preferred embodiment, each of said first and second pairs of antagonist actuation tendons comprises an opening actuation tendon 71, 73 and a closing actuation tendon 72, 74. By making the termination seats 15, 25 close to or at the respective roots 11, 12, it is possible to keep the overall dimensions small, thus favoring miniaturization. In addition, in accordance with a preferred embodiment, each termination seat 15, 25 acts as a termination seat for both antagonistic tendons of the respective pair of antagonistic tendons, helping to keep the number of processes to be performed on each root 11, 12 to a minimum, favoring miniaturization.

In accordance with an embodiment, the first termination seat 15 of the first tip 10 and the second termination seat 25 of the second tip 20 are each delimited by a cantilevered drag leg 77, 78 extending longitudinally from the respective root 11, 21 next to the elongated body of the respective tip 10, 20. Thereby each termination seat 15, 25 of the first and second tips 10, 20 is a substantially radial slot, and preferably a longitudinal slot, having a radially-facing bottom wall formed by the respective attachment root 11, 21.

Preferably, the extension of the cantilevered drag leg 77, 78 between the back side D1, D2 and the cutting side P1, P2 of the respective tip 10, 20 is substantially identical, so as to face edge surfaces of the respective termination seat 15, 25 which are placed side-by-side at the same level and which act as stop and drag abutments for the respective termination of tendons 70 of each actuation tendon 71, 72, 73, 74 of the respective pair of antagonistic tendons. The tendon termination 70 of each actuation tendon can be an enlarged portion, for example formed by a knot or a boss, which abuts against said edge walls of the respective termination seat 15, 25. In other words, said edge walls of each termination seat 15, 25 comprise edge walls formed by the respective cantilevered drag leg 77, 78 and by the elongated body of the respective tip 10, 20 facing the respective back side D1, D2 acting as closing drag edge walls, and opposite edge walls of the same respective cantilevered drag leg 77, 78 and of the elongated body of the respective tip 10, 20 facing to be opposite, i.e., facing the respective cutting side P1, P2 acting as opening drag edge walls. Therefore, the edge walls of the termination seats 15, 25 are arranged as an undercut for the respective tendon termination 70 in the respective termination seat 15, 25, and each termination seat 15, 25 is a through termination seat and preferably having an access opening facing longitudinally towards the free end 12, 22 of the respective tip 10, 20. The distal portions of each actuation tendon 71, 72, 73, 74 of said first and second pairs of antagonistic tendons therefore intersect, and/or overlap, in the respective termination seat 15, 25 to bring the respective tendon termination 70 to abut against the edge walls placed circumferentially as an undercut with respect thereto to exert the drag in rotation of the first tip or the second tip 20 in the opening and/or closing direction of the degree of freedom of opening/closing G.

In accordance with a preferred embodiment, the first root 11 of the first tip 10 and the second root 21 of the second tip 20 each comprise at least one pulley surface 79, 80 facing away from the common rotation axis Y-Y which laps the respective termination seat 15, 25 from circumferentially opposite sides and which can continue inside the respective termination seat 15, forming the radially facing bottom wall thereof, i.e., facing away from the common rotation axis Y-Y, so that a distal portion close to the respective tendon termination 70 of each of said first and second pairs of tendons 71, 72, 73, 74 winds on said at least one pulley surface 79, 80.

In accordance with a preferred embodiment, the at least one pulley surface 79 of the first root 11 and the at least one pulley surface 80 of the second root 21 are all convex ruled surfaces with parallel generatrices and parallel to the common rotation axis Y-Y which do not comprise circumferential channels or grooves for guiding or retaining the tendons. The at least one pulley surface 79, 80 can be interrupted at a radial cutting channel, where present.

In accordance with an embodiment in which the first tip 10 is made in a single piece, the first termination seat 51 is made in a single piece with the first root 11, and also the respective pulley surface 79 as well as the respective cantilevered leg 77 will be made in a single piece with said first root 11. By making the first termination seat 15 in a single piece with the first root 11, it allows keeping the number of pieces small, facilitating assembly and favoring miniaturization.

Where the first root 11 of the first tip 10 is formed by a root 31 of the blade link 30 and a root 51 of the blade holder link 50 therebetween in direct and intimate contact, i.e., where the first tip is formed by two links 30, 50, preferably the first termination seat 15 is made in a single piece with said root 51 of the blade holder link 50. In such a case, also the respective pulley surface 79 as well as the respective cantilevered leg 77 will be made in a single piece with said root 51 of the blade holder link 50. By making the first termination seat 15 in a single piece with the blade holder link 50, the number of pieces can still be kept small, thus facilitating assembly and favoring miniaturization. Therefore, the blade link 30 will have no termination seat. Thereby, it is possible to keep the number of actuation tendons small, as well as to keep the number of termination seats to a minimum, thus favoring miniaturization. Furthermore, it is possible to make the root 31 of the blade link 30 very thin, or at least as thin as the elastically bendable blade portion 14, simplifying the creation of the blade link 30 and at the same time allowing a fine characterization of the mechanical properties thereof functional to the cutting action.

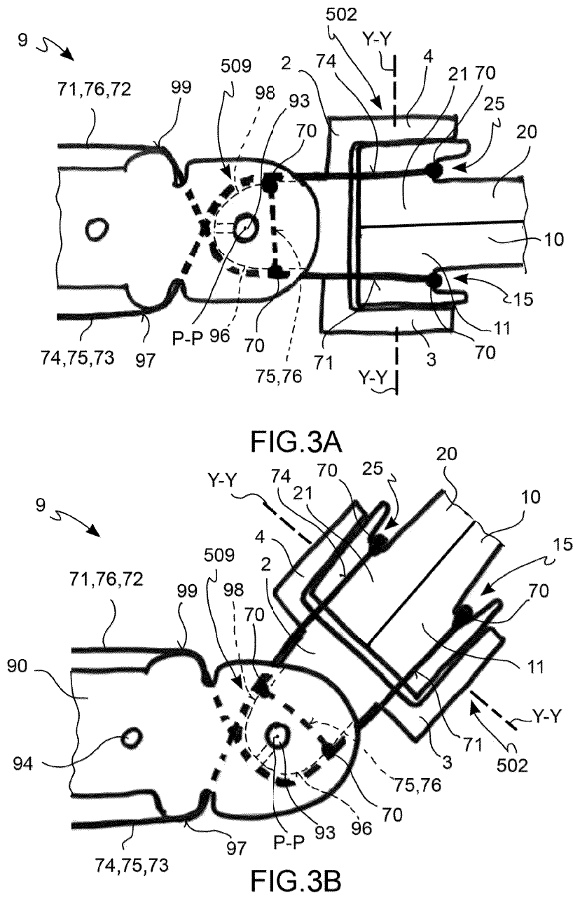

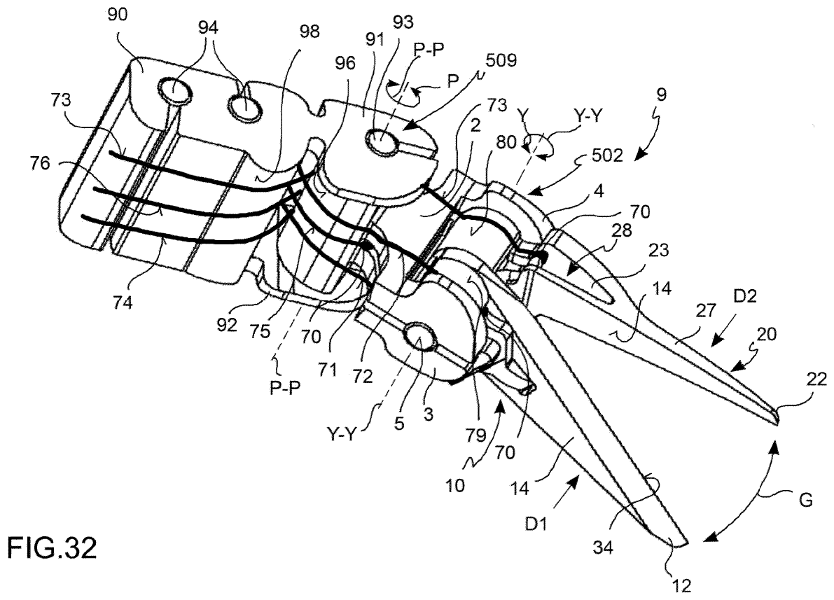

In accordance with an embodiment in which said support link 2 which is articulated with respect to the distal end 8 of the shaft 7 is provided, the surgical instrument 1 further comprises a third pair of antagonistic tendons 75, 76 for moving the support link 2 about said common proximal rotation axis P-P. Therefore, the support link 2 can comprise at least a third termination seat 6 which receives the tendon terminations 70 of said third pair of antagonistic tendons 75, 76. As shown in FIGS. 3 and 4 as well as in FIGS. 31 and 32, for example, said at least a third termination seat 6 of the support link 2 can be a single third termination seat 6 passing directly axially, i.e., parallel to the common distal rotation axis Y-Y through the body of the support link 2, which forms abutment and drag walls for the tendon terminations 70 placed as undercut for the respective actuation tendon 75, 76 of the third pair of tendons, similarly to what is explained above with reference to the first and second termination seats 15, 25. In accordance with an embodiment, the support link 2 comprises two separate and distinct third termination seats 6, one seat for each tendon 75, 76 of the third pair of antagonistic tendons.

In accordance with a preferred embodiment, the support link 2 comprises one or more convex ruled surfaces 96, 98 with parallel generatrices and all parallel to the common proximal rotation axis P-P, and the actuation tendons 71, 72, 73, 74 of the first and second pairs of antagonistic tendons slide on said one or more convex ruled surfaces 96, 98 of the support link 2 during the actuation of the first and/or second tip link 10, 20, in which said one or more ruled convex surfaces 96, 98 of the support link 2 do not comprise guide channels or grooves for receiving and guiding the tendons. The support link 2 can also comprise one or more convex ruled surfaces parallel to the common distal rotation axis Y-Y (not shown) on which the actuation tendons 71, 72, 73, 74 of the first and second pairs of antagonistic tendons slide during the actuation of the first and/or second tip links 10, 20.

The same one or more convex ruled surfaces 96, 98 with parallel generatrices and all parallel to the common proximal rotation axis P-P of the support link 2 can also act as a pulley surface for the actuation tendons 75, 76 of the third pair of antagonistic tendons, where the support link 2 is articulated with respect to the distal end 8 of the shaft 7 about the common proximal rotation axis P-P. Said one or more convex ruled surfaces 96, 98 of the support link 2 extend on opposite sides of the support link 2. In accordance with an embodiment, the pulley surface for the actuation tendons 75, 76 of the third pair of antagonistic tendons is formed by the internal surface of the termination seat 6 of the support link 2.

In accordance with an embodiment, in which said connection link 90 is provided, the link 90 comprises one or more convex ruled surfaces 97, 99 with parallel generatrices and all parallel to the common proximal rotation axis P-P, in which the actuation tendons 71, 72, 73, 74, 75, 76 of said first, second and third pairs of antagonistic tendons slide on said one or more convex ruled surfaces 97, 99 of the link 90. Said one or more convex ruled surfaces 97, 99 of the connection link 90 extend on opposite sides of the connection link 97, 99, and between the connection link 90 and the support link 2 the tendons 71, 72, 73, 74, 75, 76 of each of said first, second and third pairs of antagonistic tendons mutually cross to slide or wrap without sliding on the one or more convex ruled surfaces 96, 98 of the support link 2 facing to be opposite to the ruled surface 97, 99 of the connection link 90 on which they slide proximally. For example, said one or more convex ruled surfaces 96, 98 of the support link 2 are interposed between the prongs 91, 92 of the link 90 and are oriented to be opposite to the common proximal rotation axis P-P.

The convex ruled surfaces 79, 80, 96, 97, 98, 99 with parallel generatrices in sliding or winding contact with the tendons 71, 72, 73, 74, 75, 76 are preferably all external surfaces for the respective body of the link 2, 90 or tip 10, 20.

The actuation tendons 71, 72, 73, 74, 75, 76 are preferably polymer tendons formed by intertwined polymer fibers.

As mentioned above, in accordance with an embodiment, a surgical cutting instrument 1 comprising a rod 7 having a distal end 8 and an articulated end-effector 9 connected to the distal end 8 of the rod 7. Said articulated end-effector 9 can comprise a connection link 90 connected to the distal end 8 of the rod 7 having a body comprising in a single piece, one or more convex ruled surfaces of connection links 97, 99 with parallel generatrices, and a first distal connecting portion 13.

In accordance with an embodiment, said articulated end-effector 9 comprises a support link 2, which can be articulated to the connection link 90, having a body comprising in a single piece one or more convex ruled surfaces of support links 96, 98 with parallel generatrices. A proximal connecting portion articulated to the first distal connecting portion of the first connection link 90 can be included in the support link 2, defining a proximal rotational joint 509 for the connection link 90 and the support link 2 so that they can rotate relatively about a common proximal rotation axis P-P.

In accordance with an embodiment, said support link 2 further comprises a second distal link portion 17.

In accordance with an embodiment, said articulated end-effector 9 further comprises a blade holder link 50 articulated to the support link 2 having a body comprising in a single piece an attachment root of a blade holder link 51 having a pulley portion formed by one or more convex ruled surfaces 79 of a blade holder root with parallel generatrices, and a drag portion 57.

In accordance with an embodiment, said articulated end-effector 9 further comprises a blade link 30, integral in rotation with said blade holder link 50, having a body comprising in a single piece a cutting edge 34 and a drag counter-portion 37 engaged with said drag portion of the blade holder link 50.

In accordance with an embodiment, said articulated end-effector 9 further comprises a reaction link (for example a second tip link or a counter-blade link 60 where the counter-blade 24 is made on a separate counter-blade link 40) articulated to the support link 2 and to the assembly formed by the blade link 30 and the blade holder link 50, having a body comprising in a single piece a second attachment root 21 having a pulley portion formed by one or more convex ruled surfaces 80 with parallel generatrices.

In accordance with an embodiment, the attachment root of the blade holder link 51 and the attachment root 21 define with the second distal connecting portion 17 of the support link 2 a distal rotational joint 502 for the blade holder link 50, the reaction link and the support link 2, so 2,5 that they can rotate relatively about a common distal rotation axis Y-Y, orthogonal to said common proximal rotation axis P-P.

In accordance with an embodiment, said articulated end-effector 9 further comprises a counter-blade portion 24 integral in rotation with said attachment root 21 of the reaction link.

In accordance with an embodiment, furthermore said surgical cutting instrument 1 comprises a first pair of antagonistic tendons 71, 72 extending along the shaft 7 and connected to the blade holder link 50 for moving the blade link 30 about said common distal rotation axis Y-Y, a second pair of antagonistic tendons 73, 74 extending along the shaft 7 and connected to said reaction link to move the counter-blade portion 24 about said common distal rotation axis Y-Y, each tendon 71, 72, 73, 74 having a longitudinal extension.

In accordance with an embodiment, the attachment root of the blade holder link 50 comprises in a single piece at least a first termination seat 15 receiving said first pair of antagonistic tendons 71, 72, and the attachment root 21 comprises in a single piece at least a second termination seat 25 receiving said second pair of antagonistic tendons 73, 74.

In accordance with an embodiment, said one or more convex ruled surfaces 97, 99 with parallel generatrices of the connection link 90 are parallel to said common proximal rotation axis P-P.

In accordance with an embodiment, at least one of said convex ruled surfaces 96, 98 with parallel generatrices of the support link 2 is parallel to said common proximal rotation axis P-P.

In accordance with an embodiment, said one or more convex ruled surfaces of the blade holder root 79 with parallel generatrices of the blade-root link 50 and said one or more convex ruled surfaces of the further root 80 with parallel generatrices of the reaction link 20 are parallel to the common distal rotation axis Y-Y.

In accordance with an embodiment, the first pair of antagonistic tendons 71, 72 and the second pair of antagonistic tendons 73, 74 are adapted to slide longitudinally on said one or more convex ruled surfaces 97, 99 of the connection link 90 and on said one or more convex ruled surfaces 96, 98 of the support link 2 and are adapted to wind/unwind without sliding on the respective convex ruled surface 79 or 80 of the root of the blade holder link 50 or of the root of the reaction link, to move the blade link 30 and the counter-blade portion 24 in opening/closing, respectively.

In accordance with an embodiment, the cutting edge 34 of the blade link 30 is adapted to abut against said counter-blade portion 24 during the movement of the degree of freedom of opening/closing G in a mechanical interference contact condition to exert a cutting action, the cutting edge 34 of the blade link 30 is elastically bendable in a direction parallel to the common distal rotation axis Y-Y.

In accordance with an embodiment, a first distance Y5 in a direction parallel to the common distal rotation axis Y-Y between the first termination seat 15 of the root 51 of the blade holder link 50 and a surface 96 of said one or more convex ruled surfaces 96, 98 of the support link 2 is constant for any cutting condition.

In accordance with an embodiment, a second distance Y5′ in a direction parallel to the common distal rotation axis Y-Y between the second termination seat 25 of the second root 21 and a surface 98 of said one or more convex ruled surfaces 96, 98 of the support link 2 is constant for any cutting condition.

In accordance with an embodiment, said distal rotational joint 502 is a rigid rotational joint in the axial direction.

In accordance with one embodiment, all convex ruled surfaces 79, 80, 96, 97, 98, 99 of the links lack longitudinal channels.

In accordance with an embodiment, the attachment root 51 of the blade holder link 50 comprises a first surface facing axially outwards, and in which the second root 21 of the reaction link comprises a second surface facing axially outwards, and in which the distance Y8 in the axial direction between said first attachment root surface 51 of the blade holder link 50 and said second attachment root surface 21 of the reaction link is constant for any cutting condition.

In accordance with an embodiment, the blade holder link 50 comprises in a single piece a first cantilevered drag leg 77 extending from the root 51 of the blade holder link 50 forming a free end of the first leg 77.1, said first cantilevered drag leg 77 axially delimiting said first termination seat 15; and in which the second root 21 comprises in a single piece a second cantilevered drag leg 78 extending from the root 21 of the reaction link forming a free end of the second leg 78.1, said second cantilevered drag leg 78 axially delimiting said second termination seat 25; and in which said first and second cantilevered legs 77, 78 each comprise abutment and drag walls as an undercut with respect to the respective termination seats 15, 25 acting as dragging abutments for the respective tendon termination 70.

In accordance with an embodiment, a first distance in the axial direction between the first cantilevered leg 77 of the blade holder link 50 and a surface 96 of said one or more convex ruled surfaces 96, 98 of the support link 2 is constant for any cutting condition, and a second distance in the direction parallel to the common distal rotation axis Y-Y between the second cantilevered leg 78 and a surface 98 of said one or more convex ruled surfaces 96, 98 of the support link 2 is constant for any cutting condition.

In accordance with an embodiment, at least one of the blade holder link 50 and the blade link 30 comprises a distal free end in a single piece.

In accordance with an embodiment, the counter-blade portion 24 protrudes axially inwards, and preferably comprises an internally curved protruding surface having a concavity facing axially inwards.

In accordance with an embodiment, further comprising a third pair of antagonistic tendons 75, 76 for moving the support link 2 with said support structure about said common proximal rotation axis P-P with respect to the link 90; in which the support link 2 comprises at least a third termination seat 6 which receives the tendon terminations 70 of said third pair of antagonistic tendons 75, 76.

In accordance with an embodiment, the actuation tendons 75, 76 of said third pair of antagonistic tendons wind/unwind without longitudinally sliding on said one or more convex ruled surfaces 96, 98 of the support link 2, which therefore act as pulley surfaces for the actuation tendons 75, 76 of the third pair of antagonistic tendons.

As mentioned above, in accordance with an embodiment, the support link 2 further comprises in a single piece a proximal connecting portion 13 articulated to the first distal link portion 95 of the first connection link 90, defining a proximal rotational joint 509 for the connection link 90 and the support link 2 so that they can rotate relatively about a common proximal rotation axis P-P.

As mentioned above, in accordance with an embodiment, the support link 2 further comprises in a single piece a second distal connecting portion 17. The distal connecting portion 17 of the support structure preferably comprises a support structure, for example comprising two prongs 3, 4, for defining a distal rotation axis Y-Y, i.e., for forming a distal rotational joint 502 or yaw rotational joint 502 having a common distal rotation axis Y-Y, or yaw axis Y-Y, which can be orthogonal to the pitch proximal rotation axis P-P.

A rigid axially rotational joint 502 of a cutting joint is thus made. A blade having a cutting edge 34 and a counter-blade 24 which are integral in rotation with the axially rigid rotational joint 502 are provided, capable of jointly exerting a cutting action during the closing movement of the degree of freedom of opening/closing.

Therefore, it is possible to avoid the provision of elastic elements of the Belleville type fitted to the articulation pin 5 or otherwise interposed between the prongs 3, 4 of the distal portion 17 of the support link 2. In addition, the provision of an adjustment screw adapted to tighten the roots together in an axial direction is avoided.

Said axially rigid distal rotational joint 502 can also allow the cutting edge 34 to be oriented by rotating it about the rotation axis of yaw Y-Y, allowing control over the adjustment of the cutting orientation.

This distal rotational joint 502 is axially rigid also for any orientation of the degree of freedom of yaw Y, i.e., for any movement of the assembly formed by the blade holder links 50, the blade link 30 and the reaction link with respect to the distal portion 17 of the support link 2, as well as for any orientation of the degree of freedom of pitch P of the proximal rotational joint 509, i.e., for any movement of the assembly formed by the support link 2, and the blade holder links 50, the blade link 30 and the reaction link with respect to the connection link 90 to the shaft. Preferably, the connection link 90 to the shaft is rigidly fixed to the distal end 8 of the rod 7, for example by means of a pair of pins 94, and in this case the degree of freedom of pitch P can be understood as an orientation of the support link 2 with respect to the shaft 7 particularly where the shaft 8 is a rigid shaft.

The support structure is preferably a rigid support structure, and thereby the support link 2 with the proximal and distal connecting portions 13, 17 thereof defines in a single piece two rotational joint s 509, 502 having rotation axes P-P, Y-Y preferably orthogonal to each other.

The articulated end-effector 9 can further comprise a blade holder link 50, articulated to the support link 2 having a body comprising in a single piece an attachment root of a blade holder link 51 having a pulley portion 79 formed by one or more convex ruled surfaces 79 of blade holder root with parallel generatrices. The blade holder link 50 comprises in a single piece a proximal attachment root 51 which is articulated in said distal rotational joint 502.

The articulated end-effector 9 can further comprise a fourth blade link 30, integral in rotation with said blade holder link 50, having a body comprising in a single piece a cutting edge 34. The cutting edge 34 is adapted to perform a cutting action. The blade link 30 comprises in a single piece a proximal attachment root 31 which is articulated in said distal rotational joint 502.

As mentioned above, the distal rotational joint 502 is capable of causing a cutting action. The cutting edge 34 of the blade link 30 is adapted to abut against said counter-blade portion 24 integral in rotation with said reaction link, during the movement of the degree of freedom of opening/closing G in a mechanical interference contact condition to exert a cutting action.

The elasticity in an axial direction for obtaining the cutting action is provided at least partially by the elasticity of the blade portion 14, while the distal rotational joint 502 to which the root 31 of the blade link 30 is articulated, is axially rigid, i.e., it is not elastically loaded because relative displacements between the distal connecting portion 17 of the support link 2 and the roots 21, 31, 51 of the reaction, blade and blade holder links on the distal rotation axis Y-Y are avoided.

As mentioned above, in order to move the links of the articulated end-effector 9 about said common axes of proximal rotation P-P and/or distal Y-Y i.e., pitch P-P and/or yaw Y-Y to activate the degrees of freedom of the articulated end-effector 9, preferably the surgical instrument 1 comprises a plurality of pairs of antagonistic actuation tendons extending from the backend portion 104 to the articulated end-effector 9 through the shaft 7 and ending on at least some of the links of the articulated end-effector 9.

In accordance with a preferred embodiment, the root 51 of the blade holder link 50 comprises in a single piece a first termination seat 15 which receives a first pair of antagonistic tendons 71, 72, and the second root 21 comprises in a single piece a second termination seat 25 which receives a second pair of antagonistic tendons 73, 74. Those skilled in the art will appreciate that in this preferred embodiment, each of said first and second pairs of antagonist actuation tendons comprises an opening actuation tendon 71, 73 and a closing actuation tendon 72, 74. By creating the terminating seats 15, 25 in a single piece with the respective link, it is possible to keep the number of pieces to a minimum, facilitating assembly and favoring miniaturization. Furthermore, the root 31 of the blade link 30 is allowed to be made very thin, or at least thin as the bendable portion, elastically simplifying the creation of the blade link 30 and at the same time allowing a fine characterization of the mechanical properties thereof functional to the cutting action. In addition, in accordance with a preferred embodiment, each termination seat 15, 25 acts as a termination seat for both antagonistic tendons of the respective pair of antagonistic tendons, helping to keep the number of operations to be performed on each of the links to a minimum, favoring miniaturization. Therefore, in this case the blade link 30 does not comprise any termination seat and is dragged in rotation by the blade holder link 50. Thereby, it is possible to keep the number of actuation tendons small, as well as to keep the number of termination seats to a minimum, thus favoring miniaturization.

In accordance with an embodiment, the first termination seat 15 of the first root and the second termination seat 25 of the second root 21 are each delimited by a cantilevered drag leg 77, 78 extending longitudinally from the respective root next to the body of the respective link. Each cantilevered leg 77, 78 is preferably made in a single piece with the respective link thereof and is proximally attached to the respective root and protrudes cantilevered longitudinally alongside the body of the blade holder link 50 or the body of the reaction link, respectively, forming a leg free end 77.1, 78.1. Thereby, each termination seat 15, 25 of the blade holder link 50 and the reaction link are substantially radial slots, and preferably also longitudinal slots, having a radially-facing bottom wall formed by the respective attachment root.

Preferably, the extension of the cantilevered drag leg 77, 78 and of the respective side-by-side portion of the body of the blade holder link 50 or of the reaction link is substantially identical, respectively, so as to face abutment and drag walls 15.1, 25.1 of the edge of the respective termination seat 15, 25 which are placed side by side at the same level in the opening/closing direction and which act as abutment and drag abutments for the respective tendon termination 70 of each actuation tendon 71, 72, 73, 74 of the pair of antagonistic tendons received in the first or second termination seat 15, 25, respectively. The tendon termination 70 of each actuation tendon can be an enlarged portion, for example formed by a knot or a boss, which abuts against said abutment and drag walls 15.1, 25.1 of the edge of the respective termination seat 15, 25. In other words, said abutment and drag walls 15.1, 25.1 of the edge of each termination seat 15, 25 comprise edge walls which act as closing drag abutments, and opposite edge walls facing to be opposite which act as opening drag abutments. Therefore, abutment and drag walls 15.1, 25.1 of the termination seats 15 and 25 are arranged as an undercut for the respective tendon termination 70 in the respective termination seat 15, 25, and each termination seat 15, 25 is a through termination seat and preferably having an access opening facing longitudinally towards the free end of the respective link. Therefore, the distal portions of each actuation tendon 71, 72, 73, 74 of said first and second pairs of antagonistic tendons intersect, and/or overlap, in the respective termination seat 15, 25 to bring the respective tendon termination 70 to abut against the abutment and drag walls 15.1, 25.1 placed circumferentially as an undercut with respect thereto to exert the dragging in rotation on the blade holder link 50 and/or on the reaction link in the opening and/or closing direction of the degree of freedom of opening/closing G.

Therefore, in this case, a first axial distance Y5 can be defined as a distance in the direction of the rotation axis Y-Y between the first cantilevered leg 77 of the blade holder link 50 and a surface 96 of said one or more convex ruled surfaces 96, 98 of the support link 2, and such first axial distance is constant for any cutting condition. Likewise, in this case, a second distance Y5′ can be defined as a distance in a direction parallel to the common distal rotation axis Y-Y between the second cantilevered leg 78 and a surface 98 of said one or more convex ruled surfaces 96, 98 of the support link 2 is constant for any cutting condition. Since the axial distances Y5, Y5′ remain unchanged in any cutting condition, i.e., no sliding is provided along the articulation pin 5 of the distal rotation axis Y-Y, such distances or other axial distances can be evaluated between different points of the articulated end-effector 9. In accordance with an embodiment, the attachment root 51 of the blade holder link 50 comprises a first surface 85 facing axially outwards, and in which the further root 21 of the further reaction link comprises a second surface 86 facing axially outwards, and in which the distance Y8 in the axial direction between said first surface 85 of the attachment root 51 of the blade holder link 50 and said second surface 86 of the further attachment root 21 of the reaction link is constant for any cutting condition. The surfaces 85, 86 can be flat surfaces orthogonal to the distal rotation axis Y-Y.

In accordance with a preferred embodiment, the axial distance Y5 between the first termination seat 15 of the root 51 of the blade holder link 50 and a surface 96 of said one or more convex ruled surfaces 96, 98 of the support link 2 is equal to the axial distance Y5′ between the second termination seat 25 of the root 21 of the further reaction link and a surface 98 of said one or more convex ruled surfaces 96, 98 of the support link 2.

Therefore, avoiding axial sliding along the articulation pin 5 between the roots, as well as between the roots and the prongs, keeps the geometric relationship between the ruled surfaces 96, 98 of the support link 2 on which the tendons 71, 72, 73, 74 of the first or second pair of tendons slide longitudinally to actuate the degree of freedom of opening/closing G, i.e., to exert the cutting action and the termination seats 15, 25 for the respective tendons made in a single piece with the root 51 of the blade holder link 50 or the root 21 of the reaction link, respectively, without thereby preventing the relative rotation between said links about the common distal rotation axis Y-Y. In the direction parallel to the rotation axis the tendons do not slide with respect to the respective ruled surfaces thereof.

In accordance with a preferred embodiment, as mentioned above, the root 51 of the blade holder link 50 and the second root 21 each comprise at least one pulley surface 79, 80 facing to be opposite to the common rotation axis Y-Y which laps the respective drag seat 15, 25 from circumferentially opposite sides and which can continue inside the respective termination seat 15, 25 forming the bottom radially-facing wall, i.e., facing to be opposite to the common rotation axis Y-Y, so that a distal portion close to the respective tendon termination 70 of each tendon of said first and second pairs of tendons 71, 72, 73, 74 winds about said at least one pulley surface 79, 80 when the tendon termination 70 abuts against the abutment and drag walls 15.1, 25.1 thereof of the respective termination seat 15, 25.

In accordance with a preferred embodiment, the at least one pulley surface 79 of the root 51 of the blade holder link 50 and the at least one pulley surface 80 of the root 21 of the reaction link are all convex ruled surfaces with parallel generatrices and parallel to the common rotation axis Y-Y which do not comprise circumferential channels or grooves for guiding or retaining the tendons. The at least one pulley surface 79, 80 can be interrupted at a radial cutting channel 19, 29, where present.

In accordance with a preferred embodiment, the support link 2 comprises one or more convex ruled surfaces 96, 98 with parallel generatrices and all parallel to the common proximal rotation axis P-P, and the actuation tendons 71, 72, 73, 74 of the first and second pairs of antagonistic tendons slide longitudinally on said one or more convex ruled surfaces 84, 86 of the support link 2 during the actuation of the blade holder link 50 and/or the link 20, in which said one or more convex ruled surfaces 96, 98 of the support link 2 do not comprise guide channels or grooves for receiving and guiding the tendons. The support link 2 can also comprise one or more convex ruled surfaces parallel to the common distal rotation axis Y-Y (not shown in the figure) on which the actuation tendons 71, 72, 73, 74 of the first and second pairs of antagonistic tendons slide longitudinally during the actuation of the degree of freedom of opening/closing.

The same one or more convex ruled surfaces 96, 98 with parallel generatrices and all parallel to the common proximal rotation axis P-P of the support link 2 can also act as a pulley surface for the actuation tendons 75, 76 of the third pair of antagonistic tendons. Said one or more convex ruled surfaces 96, 98 of the support link 2 extend on opposite sides of the support link 2. In accordance with an embodiment, the pulley surface for the actuation tendons 75, 76 of the third pair of antagonistic tendons is formed by the internal surface of the termination seat 6 of the support link 2.

In accordance with an embodiment, the link 97, 99 comprises one or more convex ruled surfaces 71, 72, 73, 74, 75, 76 with parallel generatrices and all parallel to the common proximal rotation axis P-P, in which the actuation tendons 97, 99 of said first, second and third pairs of antagonistic tendons slide longitudinally on said one or more convex ruled surfaces 90 of the link 90. Said one or more convex ruled surfaces 97, 99 of the connection link 60 extend on opposite sides of the connection link 90, and between the connection link 90 and the support link 2 the tendons 71, 72, 73, 74, 75, 76 of each of said first, second and third pairs of antagonistic tendons mutually cross to slide or wrap without sliding on the one or more convex ruled surfaces 96, 98 of the support link 2 facing to be opposite to the ruled surface 97, 99 of the connection link 90 on which they slide proximally. For example, said one or more convex ruled surfaces 96, 98 of the support link 2 are interposed between the prongs 91, 92 of the link 90 and are oriented to be opposite to the common proximal rotation axis P-P.

The ruled convex surfaces 79, 80, 96, 97, 98, 99 with parallel generatrices of the links in sliding or winding contact with the tendons 71, 72, 73, 74, 75, 76 are preferably all external surfaces for the respective link.

The actuation tendons 71, 72, 73, 74, 75, 76 are preferably polymer tendons formed by intertwined polymer fibers. For example, said intertwined polymer fibers comprise high molecular weight polyethylene (UHMWPE) fibers.

In accordance with a general embodiment, a rotational joint 502 of a cutting joint having a rotation axis Y-Y is provided.

The rotational joint 502 can be a rotational joint of an articulated end-effector 9, according to any one of the embodiments described above.

The rotation axis of the rotational joint 502 can be the distal yaw rotation axis Y-Y of an articulated end-effector 9 of a surgical instrument.

The cutting joint is preferably actuated by actuation tendons 71, 72, 73, 74.

Said rotational joint 502 comprises a distal connecting portion of a support structure, for example comprising two prongs 3, 4.

Said rotational joint 502 further comprises a first attachment root 11 integral in rotation with a first free end 12 and with a blade portion 14 having a cutting edge 34 and having an elastically bendable body in the axial direction.

Said rotational joint 502 further comprises a second attachment root 21 integral in rotation with a second free end 22 and with a counter-blade portion 24;

In accordance with a preferred embodiment and as mentioned above, the first root 11 of the first tip 10 is in direct and intimate contact with the support structure and the second root 21 of the second tip 20 is in direct and intimate contact with the support structure.

In accordance with a preferred embodiment and as mentioned above, said first root 11 of the first tip 10 comprises a first axially facing external contact surface 81 and said first prong 3 comprises a first axially facing internal contact counter-surface 87.

In accordance with a preferred embodiment and as mentioned above, said second root 21 of the second tip 20 comprises a second axially facing external contact surface 82 and said second prong 4 comprises a second axially facing internal contact counter-surface 88. Preferably, said first external contact surface 81 of the first root 11, said first internal contact counter-surface 87 of the first prong 3, said second external contact surface 82 of the second root 21, and said second internal contact counter-surface 88 of the second prong 4 are all parallel to one another.

In accordance with a preferred embodiment and as mentioned above, the body of the first tip 10 is formed by two separate pieces, or links, comprising a blade link 30 having a body comprising in a single piece said blade portion 14 with said cutting edge 34 and a blade link root 31, and a blade holder link 50 having a blade holder link root 51, in which the blade link root 31 and the blade holder link root 51 are next to and in direct and intimate contact with each other, forming jointly said first root 11 of the first tip 10.

In accordance with a preferred embodiment and as mentioned above, said blade link root 31 is axially interposed between said blade holder link root 51 and the second root 21 of the second tip 20 and in direct and intimate contact therewith.

In accordance with a preferred embodiment and as mentioned above, the root integral in rotation with the blade portion 14 comprises in a single piece at least a first termination seat 15 for a first pair of antagonistic tendons 71, 72.

In accordance with a preferred embodiment and as mentioned above, the root integral in rotation with the counter-blade portion 24 comprises in a single piece at least a second termination seat 25 for a second pair of antagonistic tendons 73, 74.

In accordance with a preferred embodiment and as mentioned above, the support structure, for example a support link 2, comprises in a single piece one or more convex ruled surfaces 96, 98 with parallel generatrices on which the tendons of the first and second pairs of antagonistic tendons slide during the cutting action.

In accordance with a preferred embodiment and as mentioned above, said rotational joint 502 is rigid in the axial direction so that a first distance Y5 in the direction parallel to the common distal rotation axis Y-Y between the first termination seat 15 and a surface 96 of said one or more convex ruled surfaces 96, 98 of the support structure is constant for any cutting condition, and a second distance Y5′ in the direction parallel to the distal common rotation axis Y-Y between the second termination seat 25 and a surface 98 of said one or more convex ruled surfaces 96, 98 of the support structure is constant for any cutting condition.

In accordance with a preferred embodiment and as mentioned above, the attachment root 51 of the blade holder link 50 comprises a first surface facing axially outwards, and in which the second root 21 of the reaction link comprises a second surface facing axially outwards, and in which the distance Y8 in the axial direction between said first attachment root surface 51 of the blade holder link 50 and said second attachment root surface 21 of the reaction link is constant for any cutting condition.

In accordance with a preferred embodiment and as mentioned above, the blade holder link 50 comprises in a single piece a first cantilevered drag leg 77 extending from the root 51 of the blade holder link 50 forming a free end of the first leg 77.1, said first cantilevered drag leg 77 axially delimiting said first termination seat 15; and in which the second root 21 comprises in a single piece a second cantilevered drag leg 78 extending from the root 21 of the reaction link forming a free end of the second leg 78.1, said second cantilevered drag leg 78 axially delimiting said second termination seat 25; and in which said first and second cantilevered legs 77, 78 each comprise abutment and drag walls as an undercut with respect to the respective termination seats 15, 25 acting as dragging abutments for the respective tendon termination 70.

In accordance with a preferred embodiment and as mentioned above, a first distance in the axial direction between the first cantilevered leg 77 of the blade holder link 50 and a surface 96 of said one or more convex ruled surfaces 96, 98 of the support link 2 is constant for any cutting condition, and a second distance in the direction parallel to the common distal rotation axis Y-Y between the second cantilevered leg 78 and a surface 98 of said one or more convex ruled surfaces 96, 98 of the support link 2 is constant for any cutting condition.

Surgical instrument of the surgical scissor type

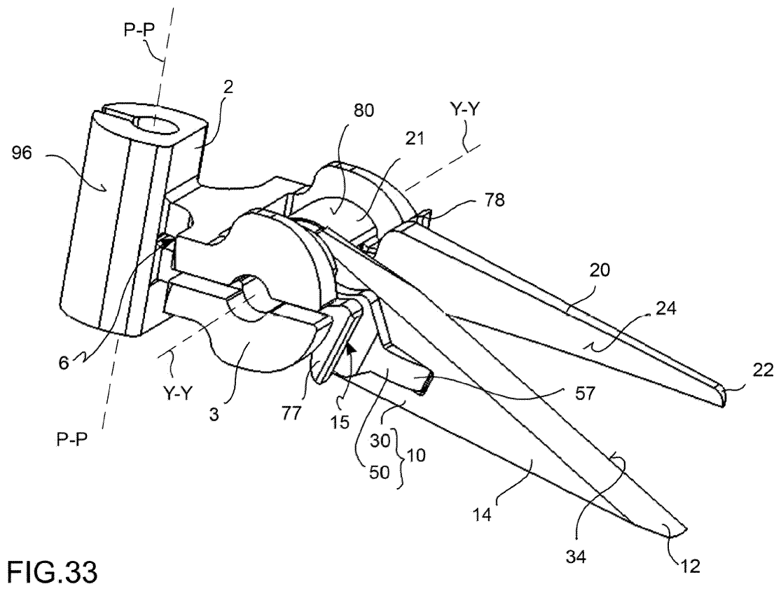

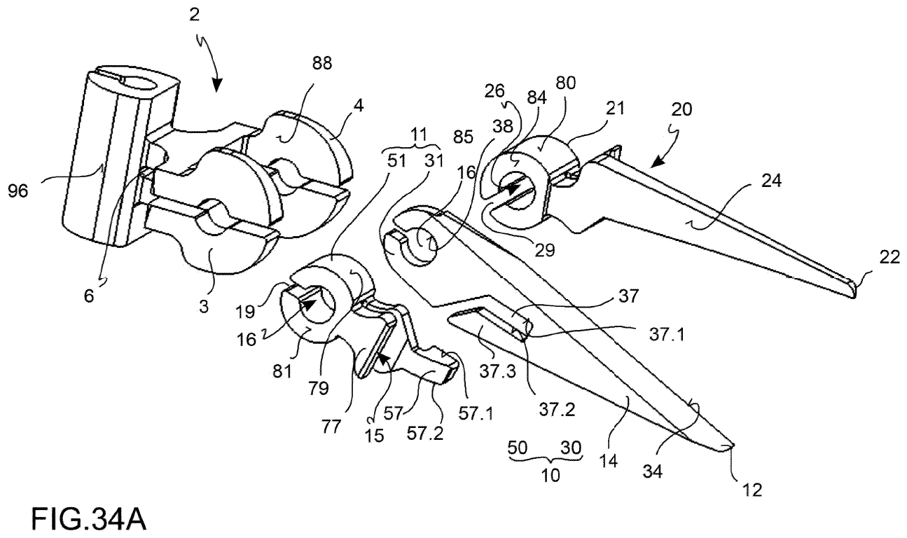

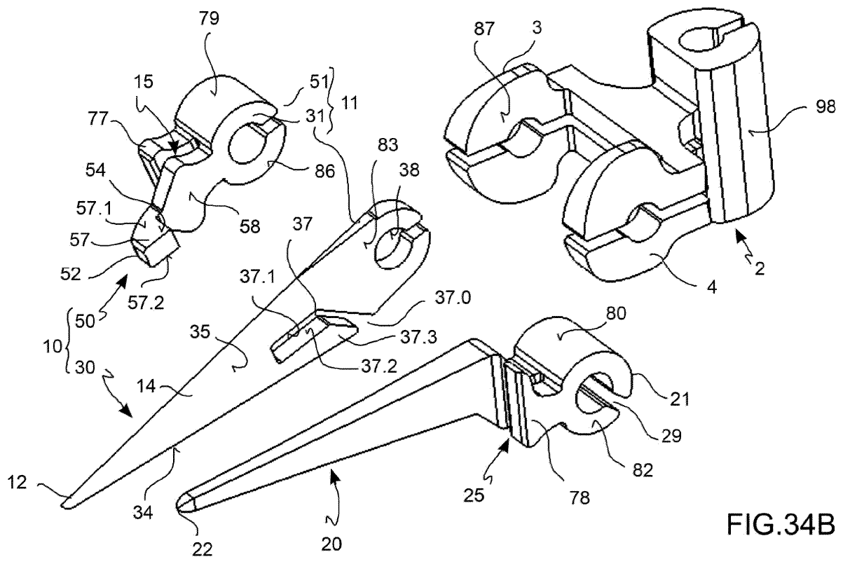

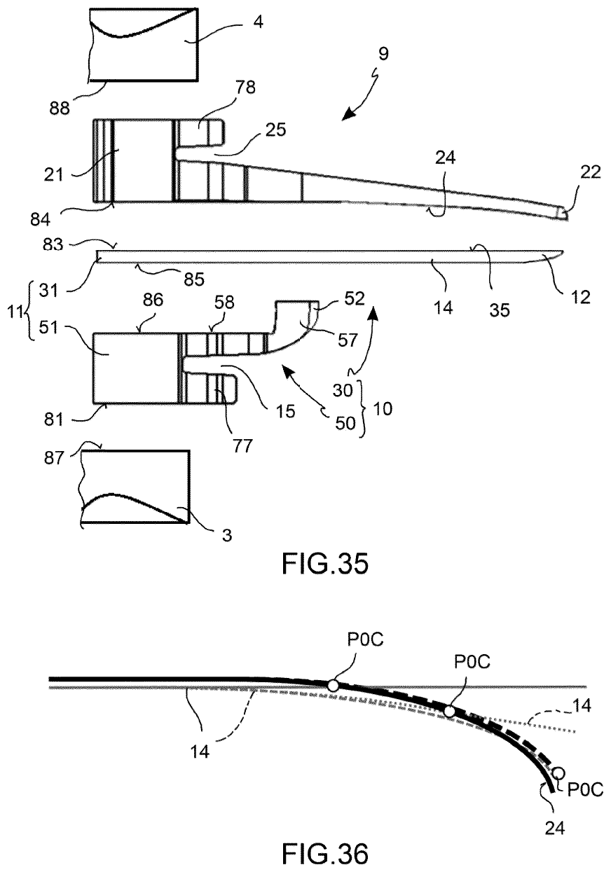

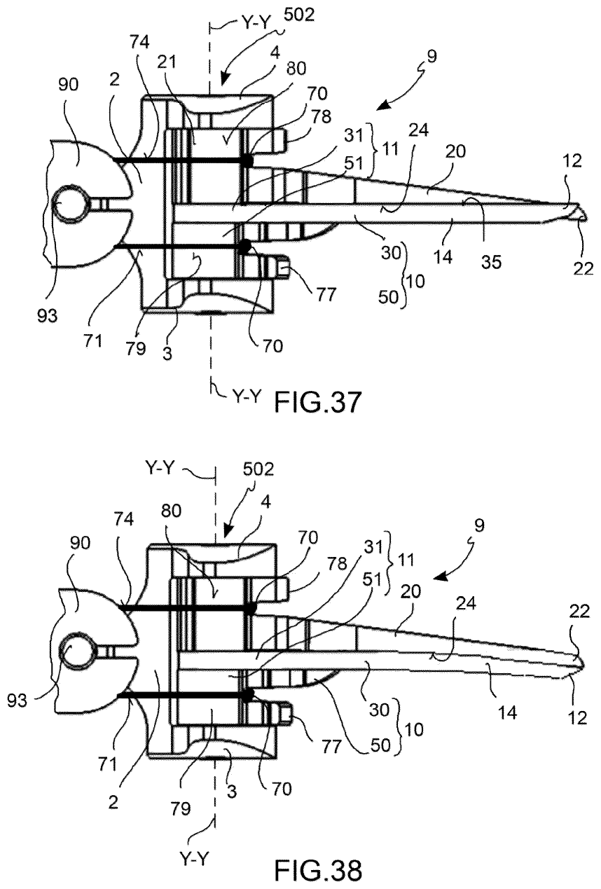

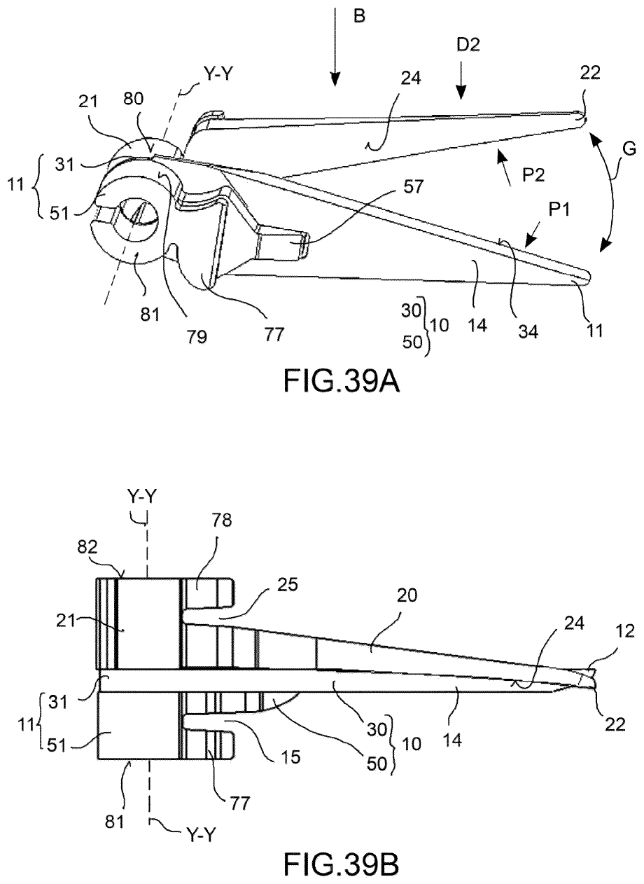

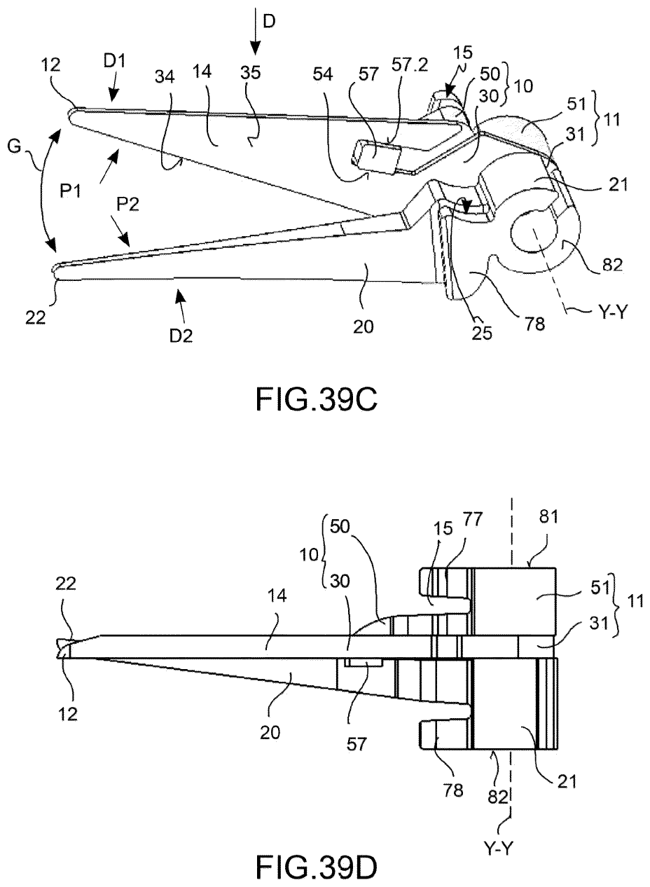

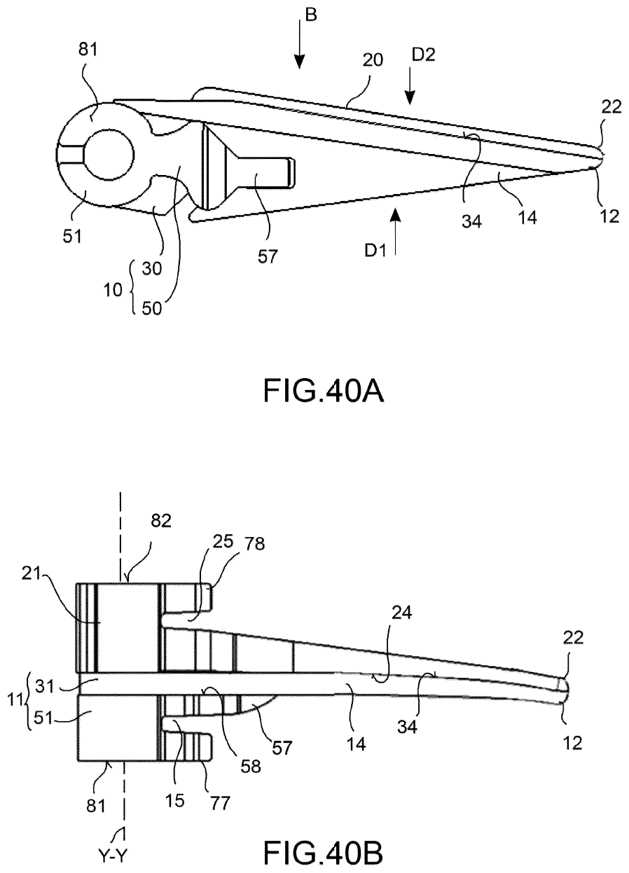

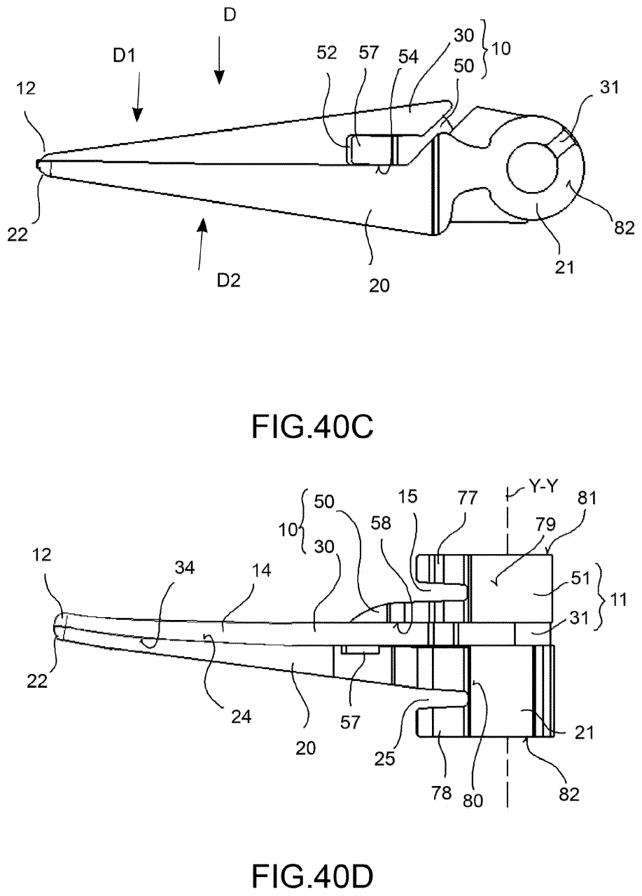

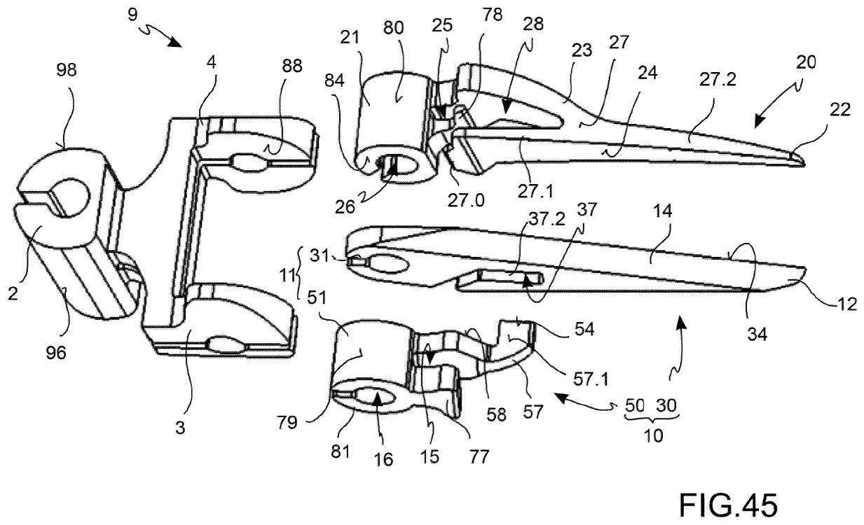

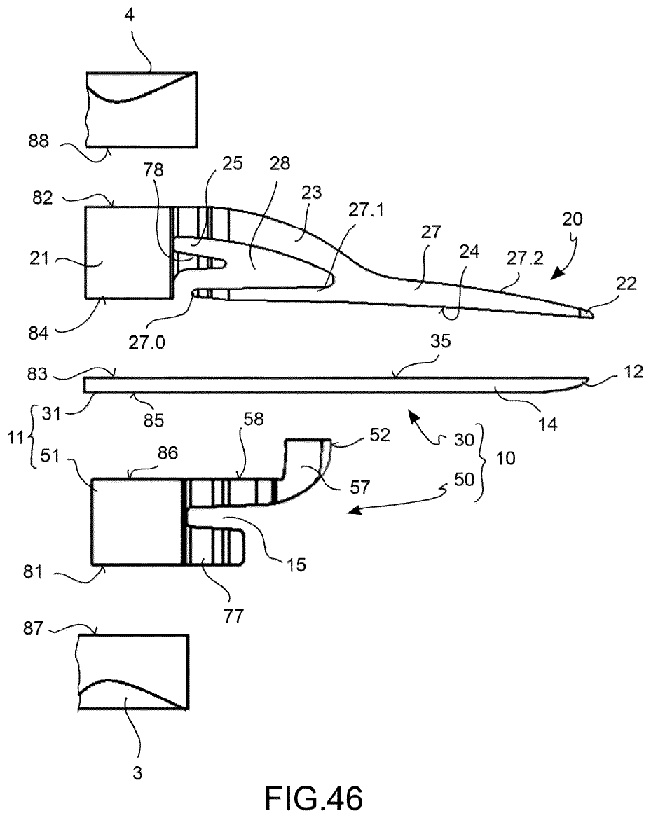

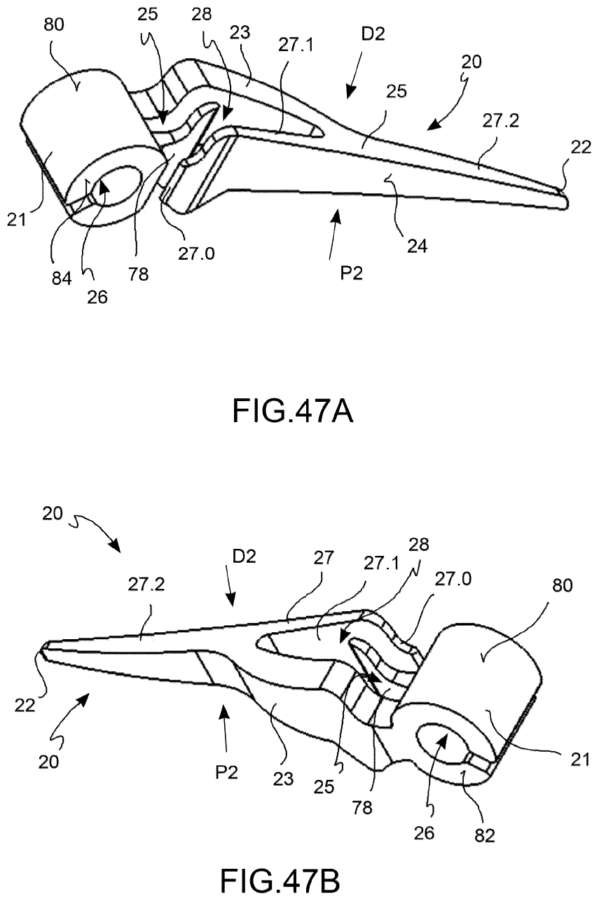

With reference to the foregoing description of embodiments of the invention, said surgical instrument 1 can be a surgical scissor type instrument as shown in FIGS. 31-53 and 55, for example. Embodiments of said surgical instrument 1 will be described below, in which said surgical instrument 1 is a surgical scissor type instrument.

In accordance with a preferred embodiment, the first free distal end 12 of the first tip 10 coincides with the distal end of the blade portion 14, although the first tip 10 can be formed by said two blade 30 and blade holder 50 links.

In accordance with a preferred embodiment, the body of the second tip 20 is also axially elastically bendable for exerting the cutting action. Therefore, during the cutting action, the mechanical interference contact between the cutting edge 34 of the blade portion 14 of the first tip 10 and the counter-blade portion 24 of the second tip 20 results in an elastic bending deformation of the blade portion 14 directed axially outwards and simultaneously results in an elastic bending deformation of the second tip 20 directed axially outwards. It should be noted that the external axial direction of the blade portion 14 of the first tip 10 is understood to be opposite to the external axial direction of the second tip 20.

As shown in the diagram in FIG. 36, for example, where the counter-blade portion 24 of the second tip 20 is a curved protruding surface with a concavity facing axially inwards, i.e., facing the blade portion 14 in which the protrusion is accentuated distally close to or at the second distal free end 22 of the second tip 20, during the cutting action and preferably with small opening angles, i.e., less than a certain threshold for example less than 5°, the point of contact POC between the cutting edge 34 and the counter-blade portion 24 is close to the free ends 12, 22 and results in an elastic bending of the external axial blade portion 14 with respect to the non-deformed configuration thereof and at the same time an elastic bending of the second external axial tip 20 with respect to the non-deformed configuration thereof. In other words, the blade portion 14 and the second tip 20 reach an equilibrium configuration for performing the cutting action at low opening angles in which the blade portion 14 of the first tip 10 and the second tip 20 both bend elastically in an external axial direction with respect to the respective non-deformed configuration.

As mentioned above, “point of contact POC” preferably means the most distal portion of the contact area between cutting edge 34 and counter-blade portion 24.

It should be noted that when the point of contact POC between the cutting edge 34 and the counter-blade portion 24 is in a more rearward position, i.e., more proximal than the configuration described above, for example for opening angles of about 10°-25°, the configuration of the second tip 20 can describe a more pronounced curvature as compared to when the point of contact POC is close to or at the second free distal end 22 (opening angles less than the threshold, for example less than 5° or less than)10°, because the second tip 20 can be more rigid proximally and more bendable distally close to or at the second free distal end 22, but this does not necessarily mean that the blade portion 14 is also deformed, i.e., bent, describing a more pronounced curvature as compared to when the point of contact POC is close to or at the second free distal end 22 (opening angles less than the threshold, for example less than 5° or less than 10°) because the curvature of the counter-blade portion 24 can be chosen so that it is more accentuated at the second free distal end 22. In accordance with a preferred embodiment, the body of the second tip 20 is tapered longitudinally, thus being axially thinner as it approaches the second distal free end 22 of the second tip 20, so as to allow the bendability of the second tip 20.

In accordance with an embodiment, the counter-blade portion 24 of the second tip 20 is a curved protruding surface with a concavity facing axially inwards, i.e., facing the blade portion 14 in which the protrusion of the counter-blade portion 24 is accentuated distally close to or at the second distal free end 22 of the second tip 20 and also the blade portion 14 of the first tip 10 is a curved protruding portion with a concavity facing axially inwards, i.e., facing the counter-blade portion 24, in which the protrusion of the blade portion 14 is accentuated distally close to or at the first distal free end 12 of the first tip 10. In other words, in this embodiment, the blade surface 35 facing axially inwards of the blade portion 14 of the first tip 10 is a concave protruding surface with a concavity facing axially inwards, i.e., towards the counter-blade portion and the protrusion becomes accentuated distally close to or at the first free distal end 12 of the first tip 10. In this embodiment, also the cutting edge 34 preferably describes a curved path with a concavity facing axially inwards.

In accordance with an embodiment in which the blade link 30 and the blade holder link 50 further comprise respective drag engagement portions 37, 57 to make the blade link 30 and the blade holder link 50 integral in rotation, the drag engagement portion 57 of the blade holder link 50 is made as an internal axial protrusion 57, i.e., an axial ridge 57 extending axially inwards comprising an opening drag surface 57.2 and an opposite closing drag surface 57.1, and in which the drag engagement portion 37 of the blade link 30 is made as an axially through slot 37 which receives said axial ridge 57 of the blade holder link 50, said axially through slot 37 delimited by an opening drag surface 37.2 in dragging contact with said opening drag surface 57.2 of the axial ridge 57 of the counter-blade link 50 and an opposite closing drag surface 37.1 in dragging contact with said closing drag surface 57.2 of the axial ridge 57 of the counter-blade link 50. In order to obtain the assembly between blade link 30 and blade holder link 50 to determine the drag engagement, the axially through slot 37 of the blade link 30 can describe a shaped path so as to have an inlet opening 37.0 which opens on one side of the blade link 30 opposite to the cutting edge 34, i.e., which opens on the back side D1 of the blade link 30 of the first tip 10, and in which the path of the slot 37 comprises a shaped inlet channel, for example oriented in an incident direction with respect to the drag opening surface 37.2, so that said drag opening surface 37.2 is as an undercut with respect to the inlet opening 37.0 facing the back side D1. Therefore, in this case, the axial ridge 57 of the blade holder link 50 is inserted in the slot 37 of the blade link 30 by the inlet opening 37.0, and then runs through the inlet channel and is then rotated with respect to the blade link 30 so as to obtain the drag engagement.

In other words, in this case, the blade 30 comprises an opening drag leg 37.3 extending cantilevered in a longitudinal direction, for example directed proximally towards the common rotation axis Y-Y, and which does not work to obtain the cutting action, in which said cantilevered opening drag leg 37.3 comprises said opening drag surface 37.2 and delimits the inlet opening 37.0 with an edge thereof.

The axial ridge 57 of the drag engagement of the blade holder link 50 can be obtained at the distal end 52 of the blade holder link 50. Thereby, the blade holder link 50 has a squat conformation with an enlarged and/or bent distal end 52 which forms said axial ridge 57.

Not necessarily, during the cutting action in which the blade portion 14 elastically bends the blade link 30 axially slides externally with respect to the axial ridge 57 of the blade holder link 50 because the bending deformation of the blade link 30 in an axial external direction can occur only distally with respect to said drag engagement slot 37, the blade holder link 50 can comprise a surface 58 facing axially inwards between the root 51 thereof and the axial ridge 57 which is in contact with the blade link 30.

The position of the axial ridge 57 of the blade holder link 50 as well as the extension thereof in the internal axial direction can be chosen so that an axially internal portion of the axial ridge 57 with respect to the closing drag surface 57.1 forms a closing stroke end surface 54 for the second tip 20, adapted to abuttingly receive a surface of the cutting side P2 of the second tip 20 acting as a closing stroke end for the degree of freedom of opening/closing G. Therefore, the axial ridge 57 of the blade link 30 can perform both the function of making the drag engagement with the blade link 30 and the function of making the closing stroke end abutment.

Preferably, the closing stroke end surface 54 extends at a longitudinal level along the elongated body of the first tip 10 in which the cutting edge 34 is already present, i.e., the closing stroke end surface 54 faces the cutting side P1 of the first tip 10 and extends axially cantilevered from the blade surface 35 between the cutting edge 34 and the back side D1 of the first tip 10.

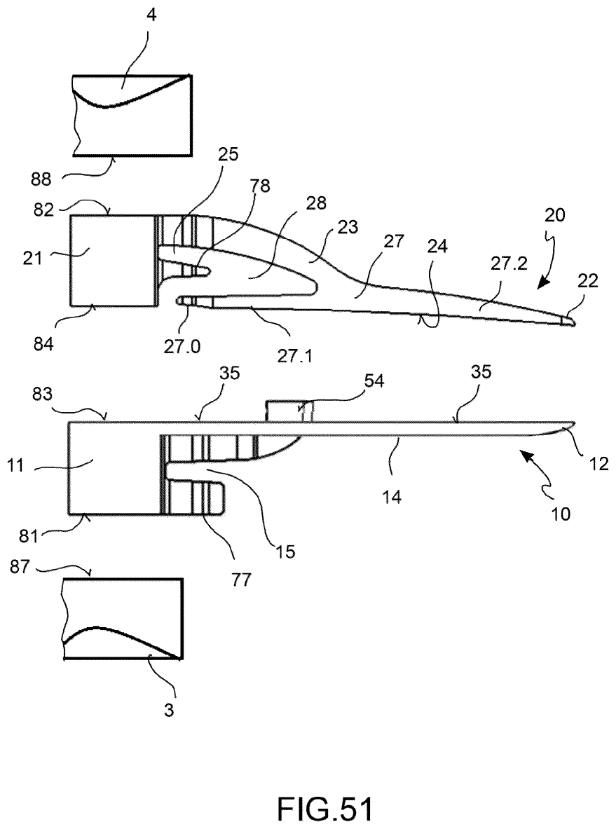

In accordance with an embodiment as shown in FIG. 43, for example, as well as in FIG. 51, for example, in which the first tip 10 is made in a single piece forming a first tip link, a closing stroke end abutment is provided, which extends axially cantilevered from the blade surface 35 between the cutting edge 34 and the back side D1 of the first tip 10, in which said closing stroke end abutment comprises a closing stroke end surface 54 adapted to abuttingly receive a surface of the cutting side P2 of the second tip 20 acting as a closing stroke end of the degree of freedom of opening/closing G.

The closing stroke end surface 54 preferably extends from the first tip 10 in the rotational approaching footprint of the second tip 20.

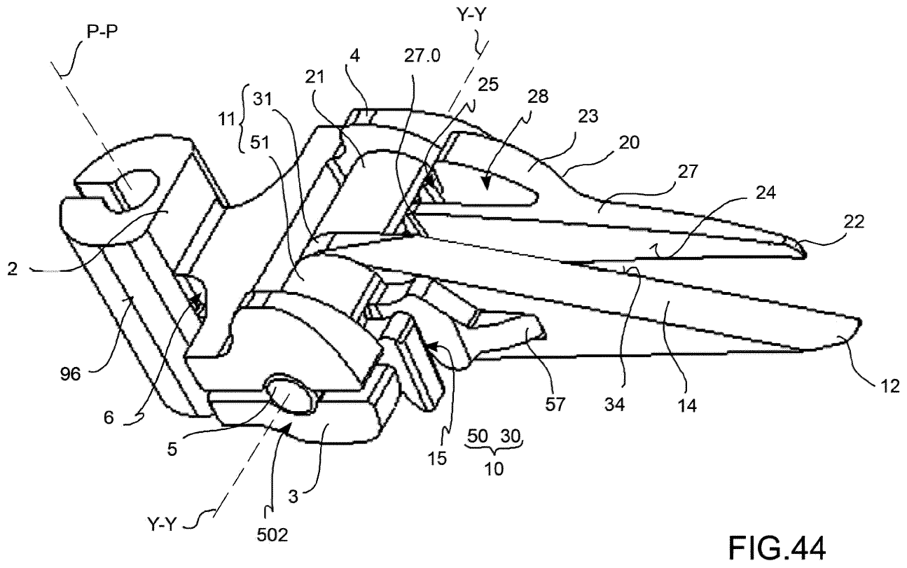

In accordance with an embodiment as shown in FIG. 44, for example, the elongated body of the second tip 20 is elastically bendable in the axial direction to exert the cutting action, in which the body of the second tip 20 comprises a connecting stem 23 extending from the second root 21 in the distal direction and ending in a cutting interface portion 27 of the body of the second tip 20, in which said cutting interface portion 27 has an elongated body directed longitudinally and axially inwards comprising two longitudinally opposite free ends and said counter-blade portion 24 therebetween. Preferably, the distal free end of the cutting interface portion 27 coincides with said second distal free end 22 of the second tip 12 and the opposite proximal free end 27.0 of the cutting interface portion 27 extends cantilevered towards the common rotation axis Y-Y, i.e., towards the second root 21 of the second tip 20. Thereby, the connecting stem 23 and the interface cutting portion 27 of the second tip 20 form a sort of “T” structure in which two cantilevered arms 27.1 and 27.2 protrude longitudinally opposite from the distal top of the connecting stem 23 of the cutting interface portion 27 each having a free end, and in which the counter-blade portion 24 belongs to both arms 27.1 and 27.2 of the cutting interface portion and faces to be opposite to the connecting stem 23.

Thereby, a counter-blade deformation seat 28 is formed between the proximal arm 27.1 of the cutting interface portion 27 and the connecting stem 23 to receive the axial deformation of the counter-blade portion 24. i.e., of the proximal arm 27.1 of the cutting interface portion 27 with the proximal free end 27.0 thereof. In accordance with an embodiment, the second termination seat 25 for the second pair of antagonistic actuation tendons 73, 74 is placed axially between the connecting stem 23 and the proximal arm 27.1 of the cutting interface portion 27. In accordance with an embodiment, the distal cantilevered leg 78 of the second termination seat 25 extends distally cantilevered between the connecting stem 23 and the proximal arm 27.1 of the cutting interface portion 27, so that the connecting stem 23 axially externally delimits the second termination seat 25 of the second tip 20 and so that the distal cantilevered leg 78 of the second termination seat 25 axially externally delimits at least a portion of the counter-blade deformation seat 28. In accordance with an embodiment, the second termination seat 25 opens into said counter-blade deformation seat 28 and therefore in this embodiment the antagonistic actuation tendons 73, 74 can be inserted in the respective second termination seat 25 which opens in the distal direction, after having axially inserted them in the opening formed between the proximal free end 27.0 of the proximal arm 27.1 of the cutting interface portion 27 and the second root 21 and after having moved them inside the counter-blade deformation seat 28 in a distal direction along the axially internal portion of the cantilevered leg 78 to then insert them in the inlet in the second termination seat 25, and thus in this embodiment the assembly of the antagonistic actuation tendons 73, 74 is preferably performed when the first tip 20 and the second tip 10 form an opening angle (e.g., opening angle of about 90°) such that the counter-blade portion 24 of the proximal arm 27.1 of the body of the second tip 20 is out of contact with the cutting edge 34 of the blade portion 14 of the first tip 10, freeing the axial access at the opening formed between the proximal free end 27.0 and the second root 21.

By virtue of the provision of such a second tip 20 comprising said connecting stem 23 ending in said cutting interface portion 27, in which said counter-blade portion 24 belonging to said cutting interface portion 27 and having a proximal arm 27.1 with a proximal free end 27.0 and a longitudinally opposite distal arm 27.2 having a distal free end coincident with said second free end 22 of the second tip 20, it is possible to make a second elastically bendable tip 20 in an external axial direction substantially along the entire longitudinal extension of the counter-blade portion 24, thus allowing a precise cutting action to be exerted even for high opening angles, for example opening angles in the range of 25°-60° and preferably in the range of 28º−58°, which correspond to a situation in which the point of contact POC belongs to said proximal arm 27.1 of the cutting interface portion, and preferably is close to or at the free proximal end 27.0 of the cutting interface portion 27. In this case and at high opening angles, the blade portion 14 does not necessarily bend elastically to exert the cutting action and the elasticity can only be conferred by the second tip 20. In particular, in accordance with an embodiment, when the point of contact POC is at the proximal free end 27.0 of the proximal arm 27.1 the opening angle is about 58° and a cutting action is still exerted.

Therefore, by virtue of the provision of such a second tip 20 comprising said connecting stem 23 ending in said cutting interface portion 27, it is possible to create a solution adapted to make a precise cut for opening angles in the range of 0°-60°, while keeping the actuation forces of the first tip 10 and the second tip 20 exerted by means of tensile action on the respective actuation tendons to a minimum, and at the same time it is possible to keep the radius of the pulley surface 79, 80 of the respective root 11, 21 at the tendon termination to a minimum, thus simultaneously allowing extreme miniaturization.

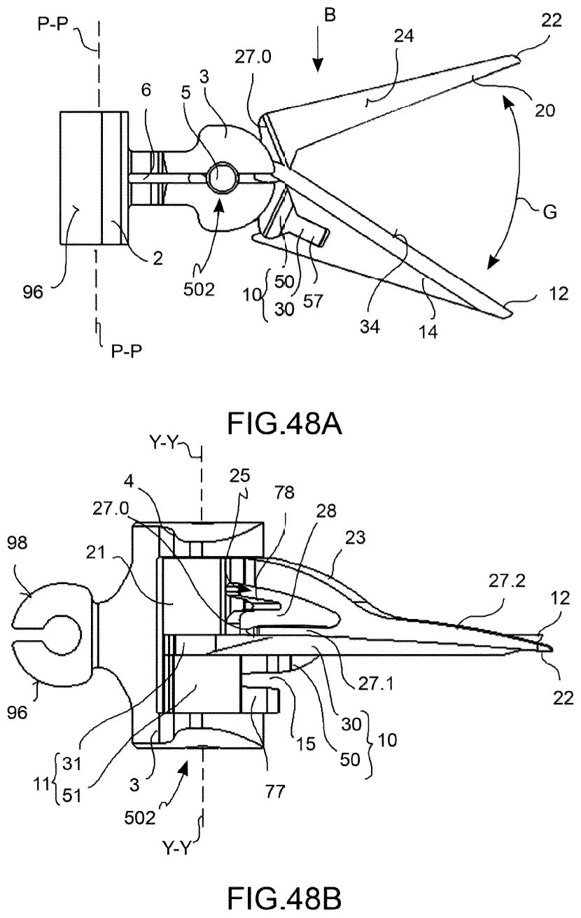

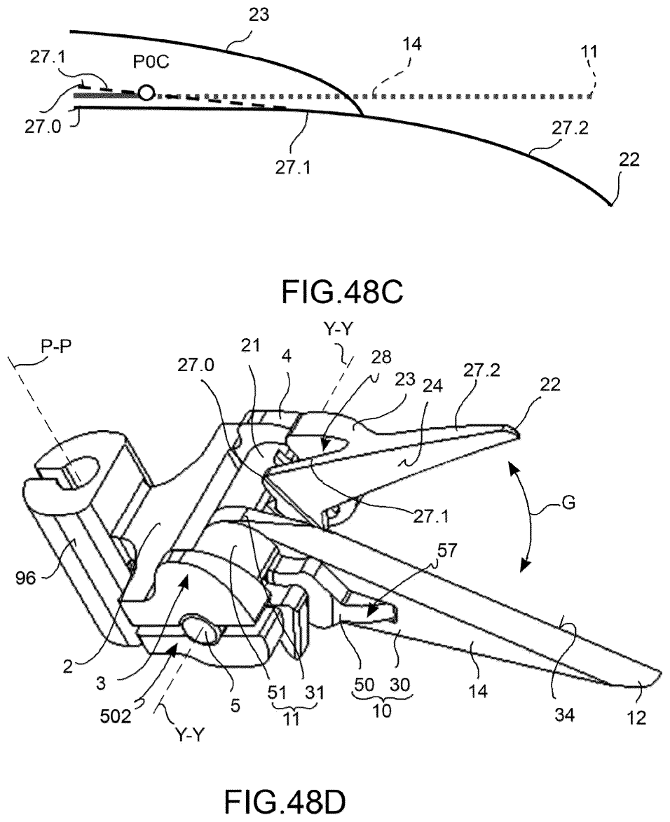

As diagrammatically shown for example in FIG. 48C, for relatively high opening angles (e.g., angle in the range of)50°-60°, the contact between the cutting edge 34 of the blade portion 14 of the first tip 10 with the counter-blade portion 24 occurs in a portion of the proximal arm 27.1 close to or at the proximal free end 27.0 of the cutting interface portion 27 of the second tip 20, and the cutting mechanical interference contact thus results in the external axial deformation of the proximal arm 27.1 inside the deformation seat 28 of the second tip 20, while the blade portion 14 of the first tip 10 remains substantially deformed, i.e., does not elastically bend because it is axially externally supported for example by the blade holder link 50 (where the first tip 10 is in a single piece, the proximal portion of the blade portion 14 is axially supported externally by the proximal portion of the tip link body 10 between the root 11 thereof and the stroke end surface 54). This allows exerting the cutting action even for high opening angles, for example opening angles up to about 60°.

As the opening angle decreases, the point of contact POC moves in the distal direction.

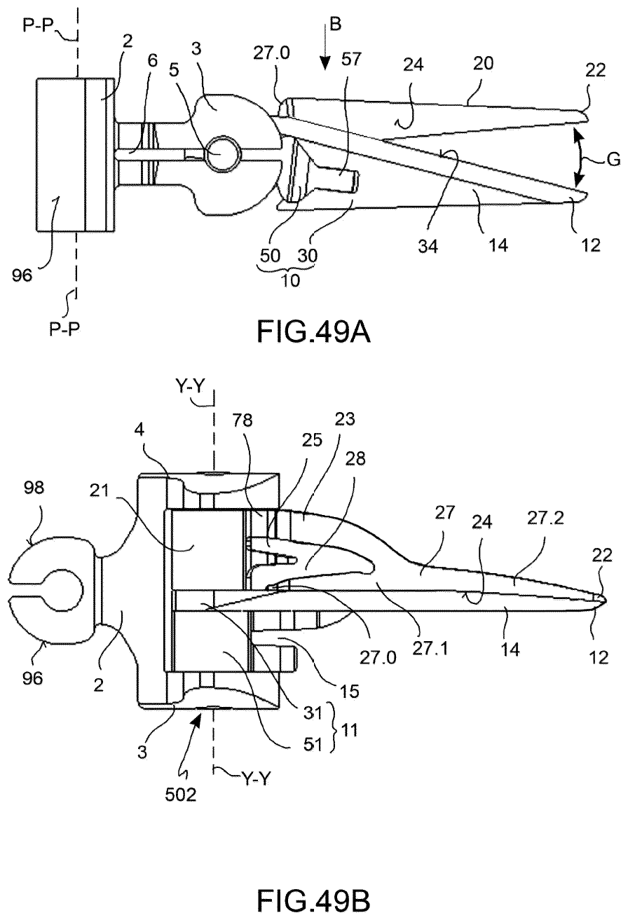

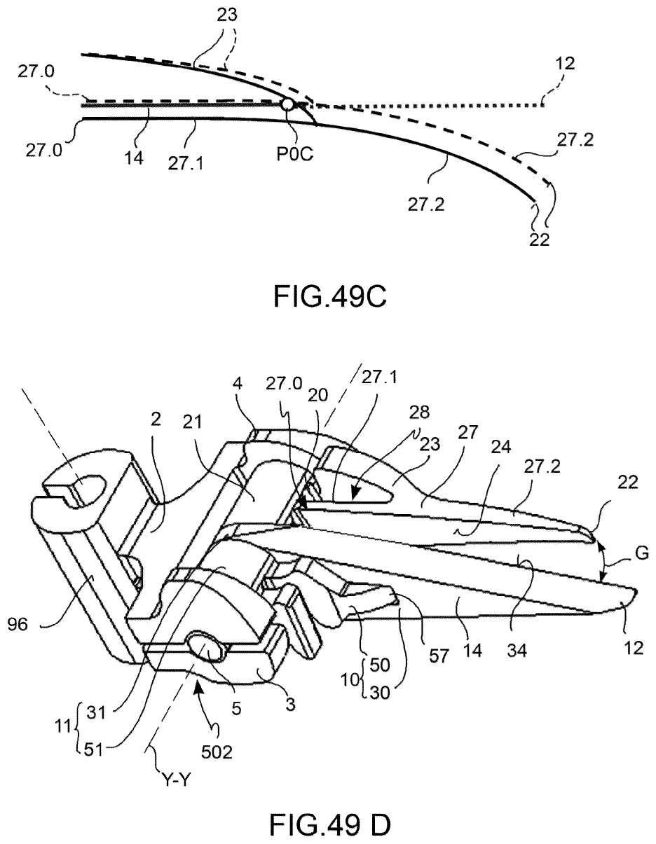

As diagrammatically shown for example in FIG. 49C, for smaller opening angles than above, i.e., for example opening angles in the range of 10°-25°, the point of contact POC between the cutting edge 34 of the blade portion 14 of the first tip 10 with the counter-blade portion 24 is in a portion of the cutting interface portion 27 of the second tip 20 close to or at the portion in which the connecting stem 23 ends, and the cutting mechanical interference contact results in the external axial deformation of the connecting stem 23 which carries the cutting interface portion 25 back in the axially external direction, while the blade portion 14 of the first tip 10 can not even bend elastically but preferably bends axially outwards anyway, especially in case of extreme miniaturization of the pieces. This allows exerting the cutting action by utilizing the external axial deformation of the second tip 20 for intermediate opening angles, for example in the range of 10°-25°. In such a case, a proximal portion of the blade portion 14 of the first tip 10 can still be in interference contact with the counter-blade portion 24 of the proximal arm 27.1 of the cutting interface portion 27 of the second tip 20.

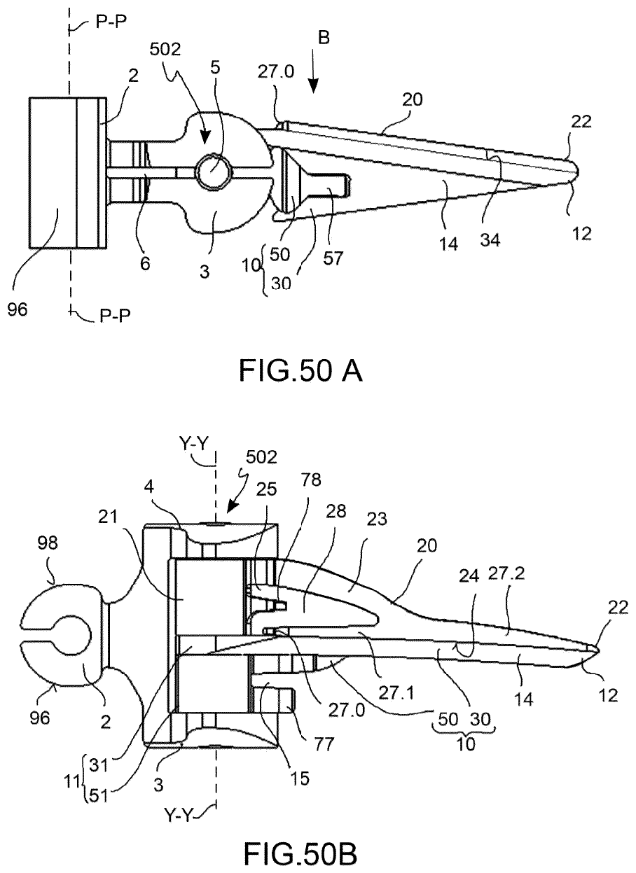

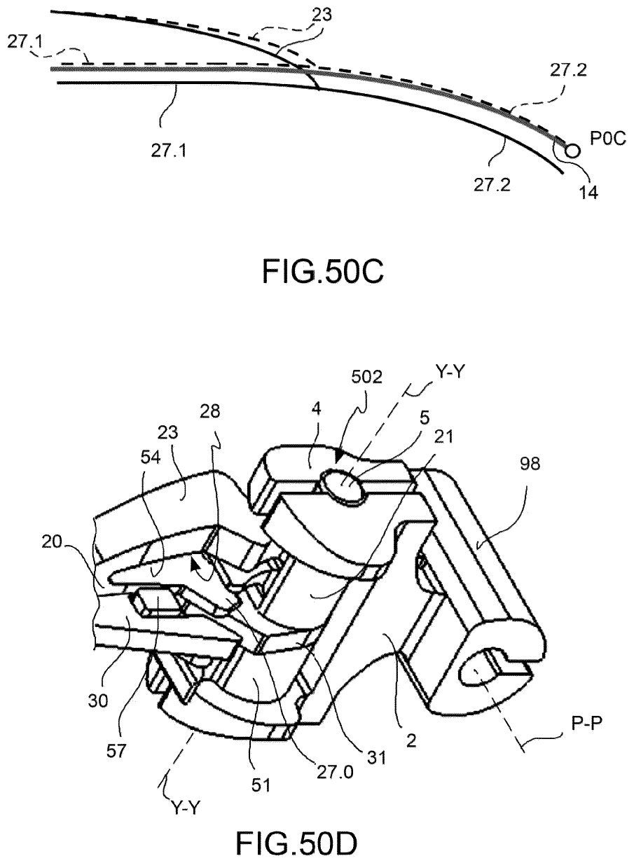

As diagrammatically shown for example in FIG. 50C, for small opening angles, for example in the range of 0°-5° and/or 0°-10°, the point of contact POC between the cutting edge 34 of the blade portion 14 of the first tip 10 with the counter-blade portion 24 is close to or at the distal free ends 12, 22 of the first and second tips 10, 20 and the cutting mechanical interference contact results in the external axial deformation of both the blade portion 14 of the first tip 10 and the cutting interface 27 and the connecting stem 23 of the second tip 20.

The curvature of the counter-blade portion 24 as well as the structure and elastic properties of the cutting interface portion 27 and the connecting stem 23 can be chosen to optimize the cutting performance for an unusually wide range of opening angles, for example in the range of 0°-60°.

The second tip 20 formed by two separate pieces or links mutually integral in rotation with each other can be provided, in which a first link of the second tip 20 comprises the counter-blade portion 24 and a second link of the second tip 20 comprises a port counter-blade holder portion, 24 and preferably both said two links comprise a root, i.e., a root of the counter-blade link and a root of the counter-blade holder link next to each other jointly form said second root 21 of the second tip 20.

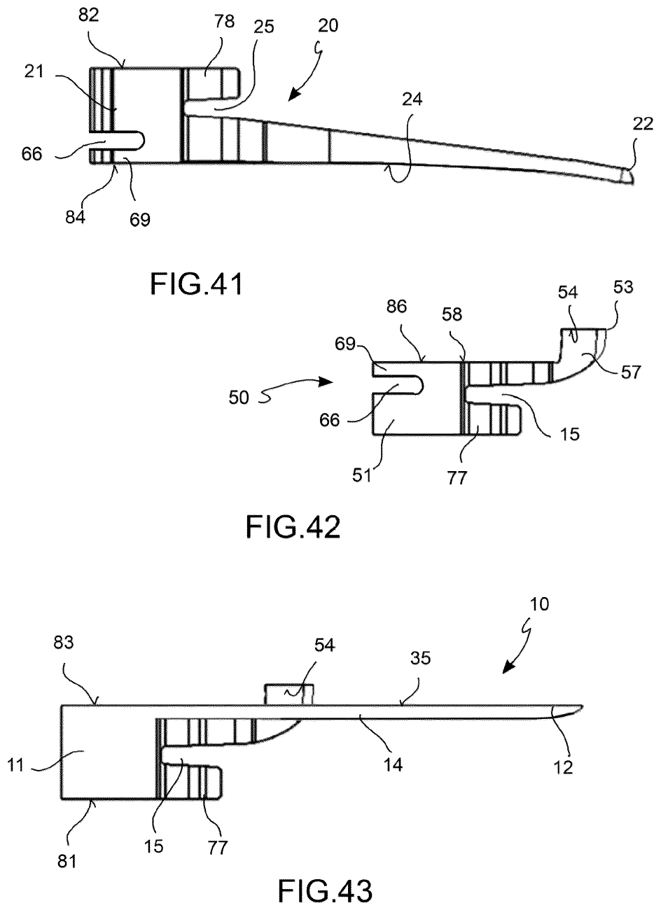

In accordance with an alternative embodiment, one or more notches 66 can be provided in the first root 11 of the first tip 10 and/or in the second root 21 of the second tip 20 to provide axial elasticity to the respective roots. As shown in FIG. 41, for example, a longitudinally directed notch 66 is provided, for example, in the proximal part of the second root 21 of the second tip 20 forming an elastic leg 69 from the side facing axially inwards of the second root 21, in which said elastic leg 69 is adapted to provide an elastic action on the first root 11 during the cutting action. As shown in FIG. 42, for example, a longitudinally directed notch 66 can be provided in the proximal part of the root 51 of the blade holder link 50 forming an elastic leg 69 from the side facing axially inwards of the root 51 of the blade holder link 50, in which said elastic leg 69 is adapted to provide an elastic action on the root 31 of the blade link 30 during the cutting action.

Needle-driver/sutures-cutter type surgical instrument

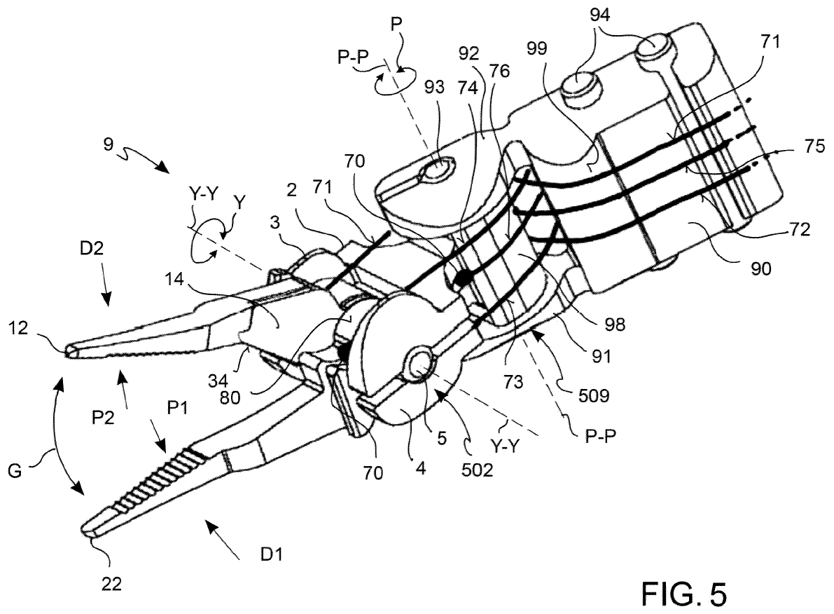

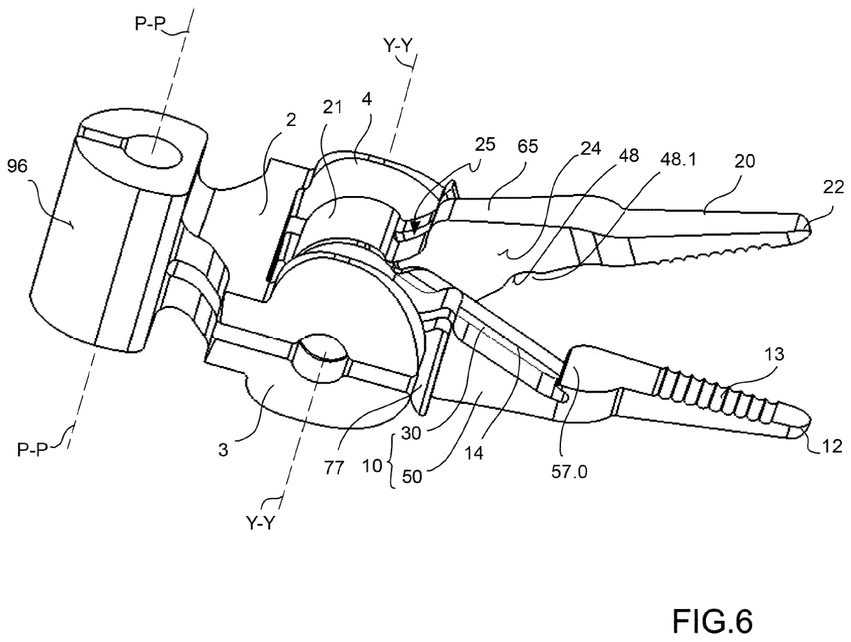

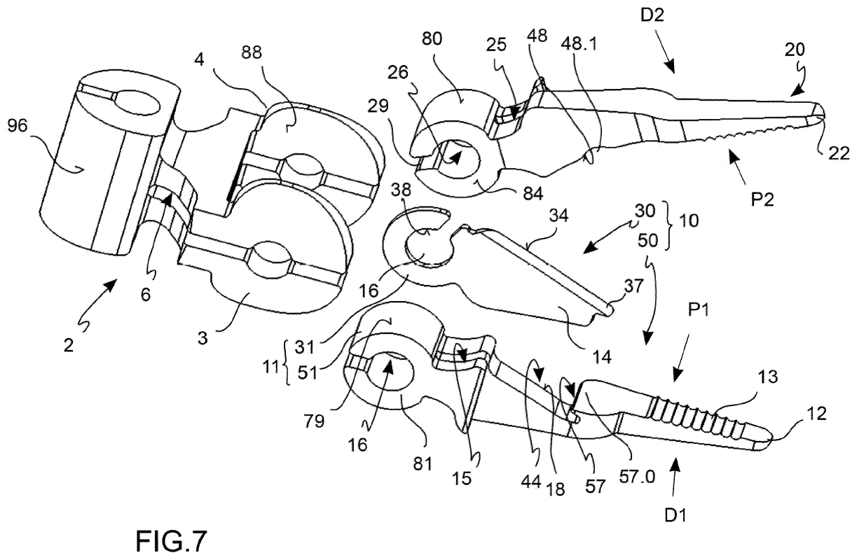

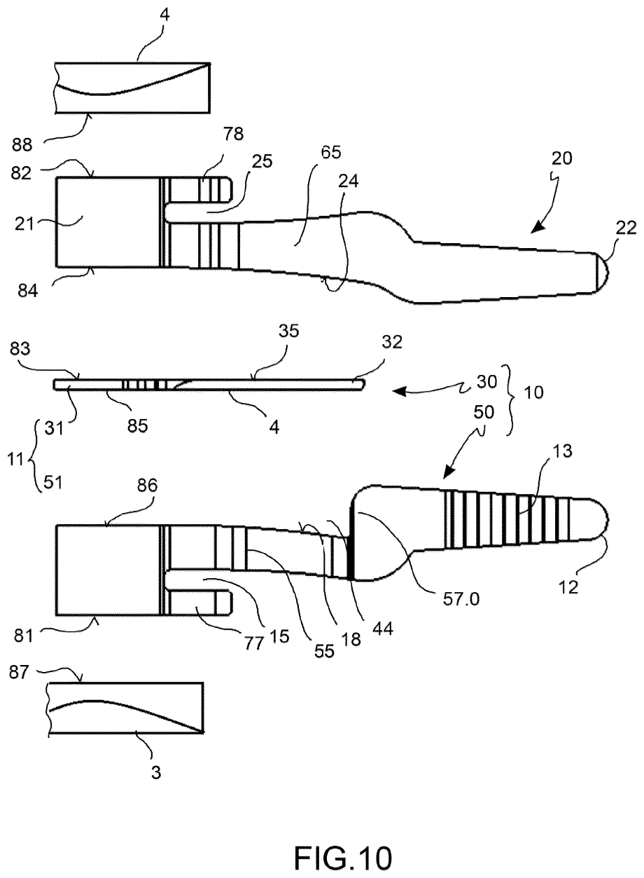

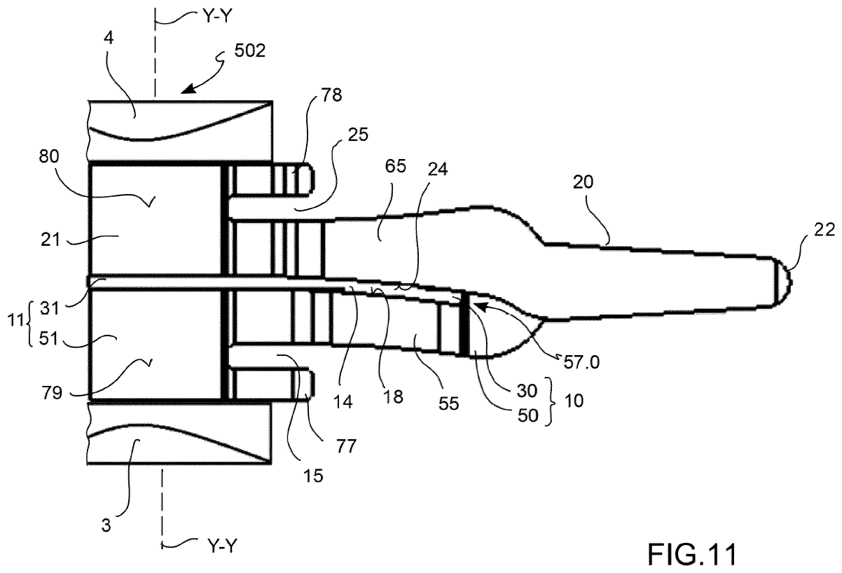

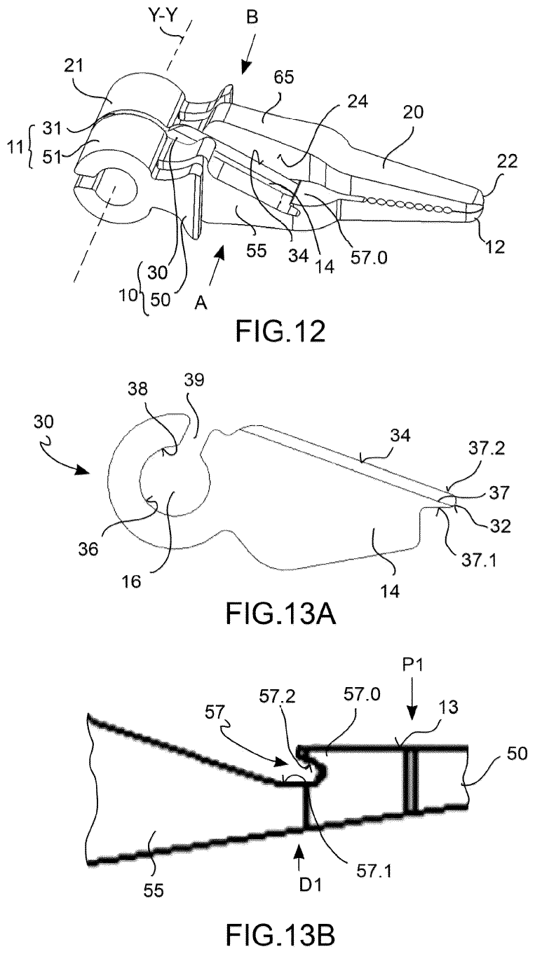

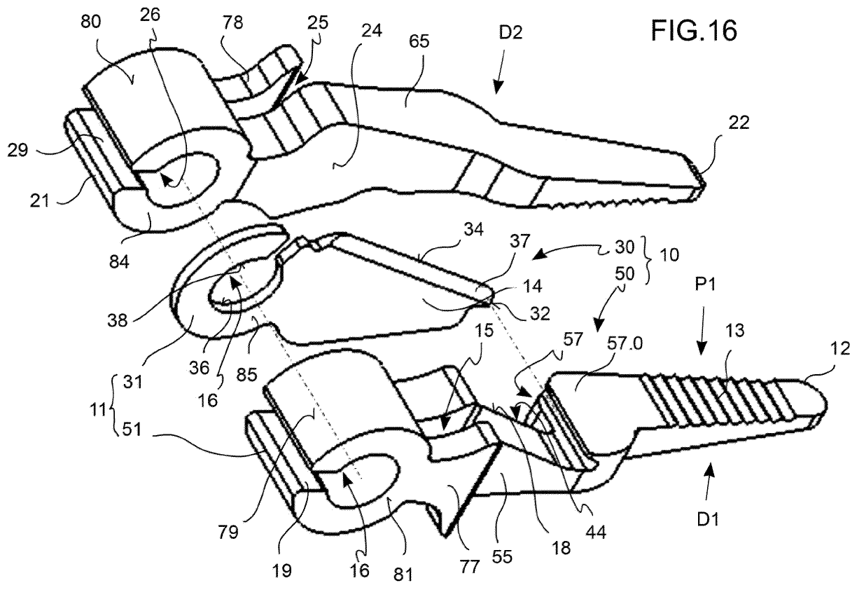

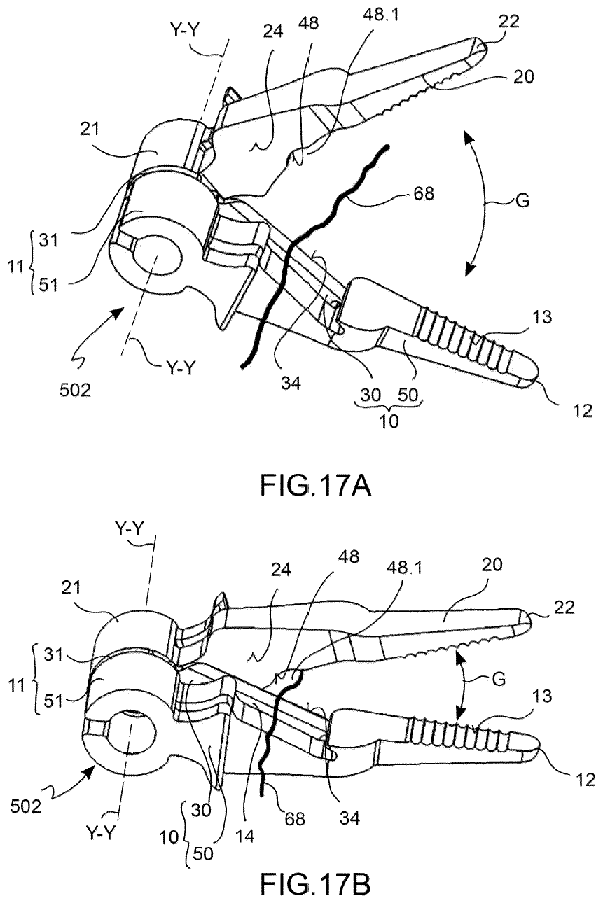

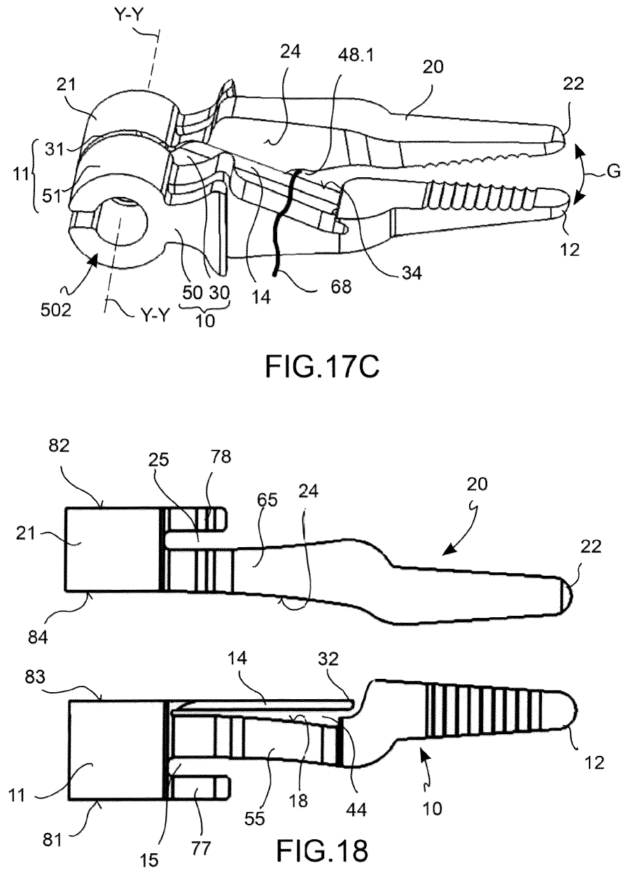

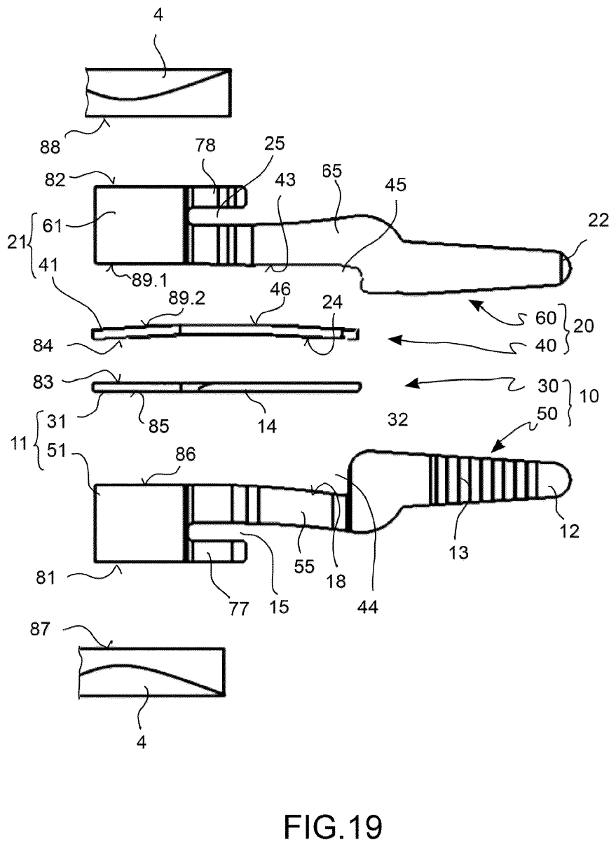

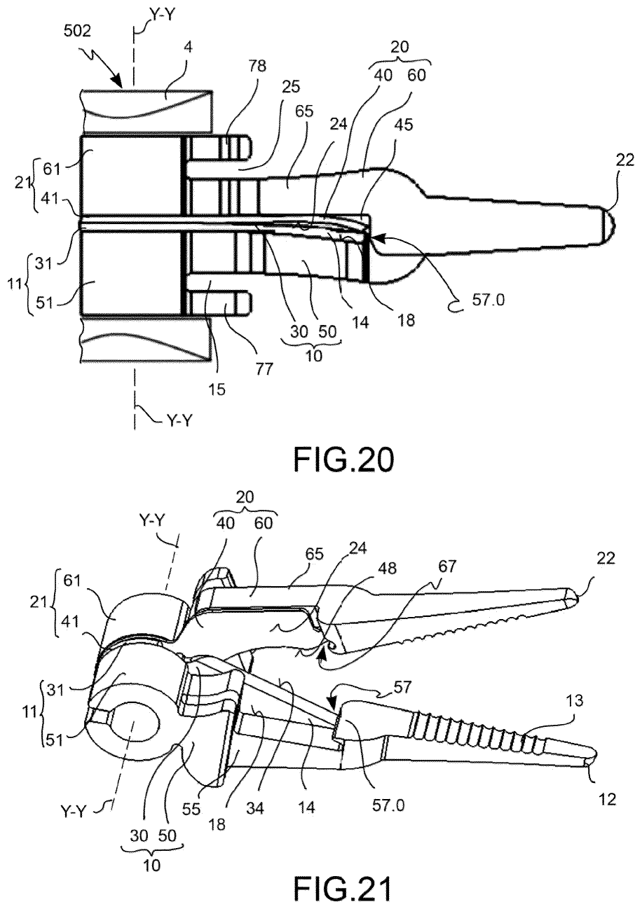

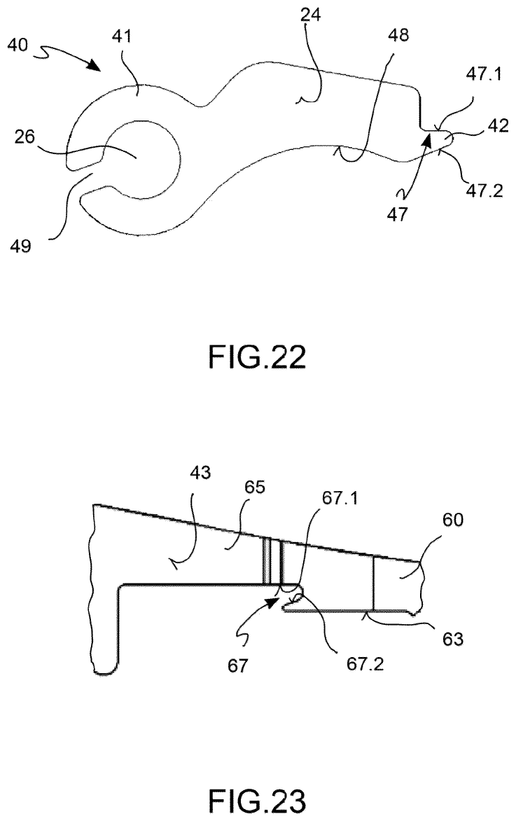

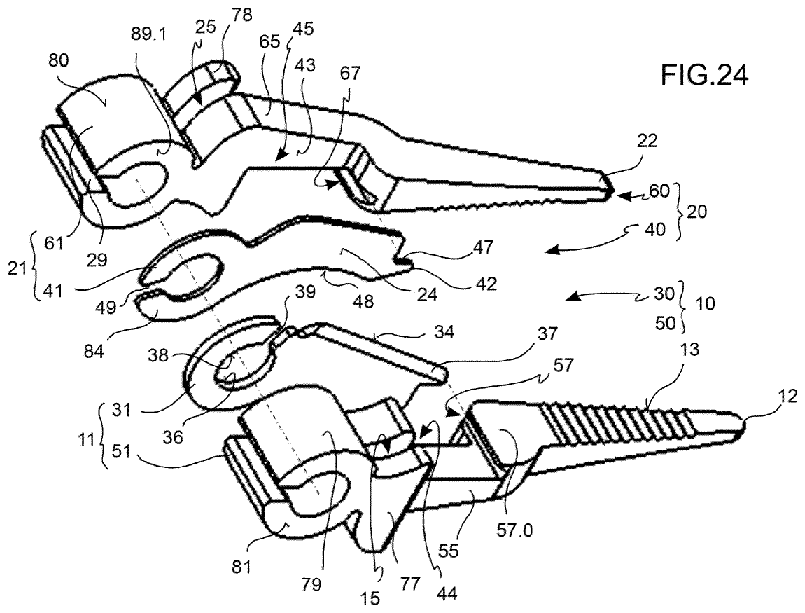

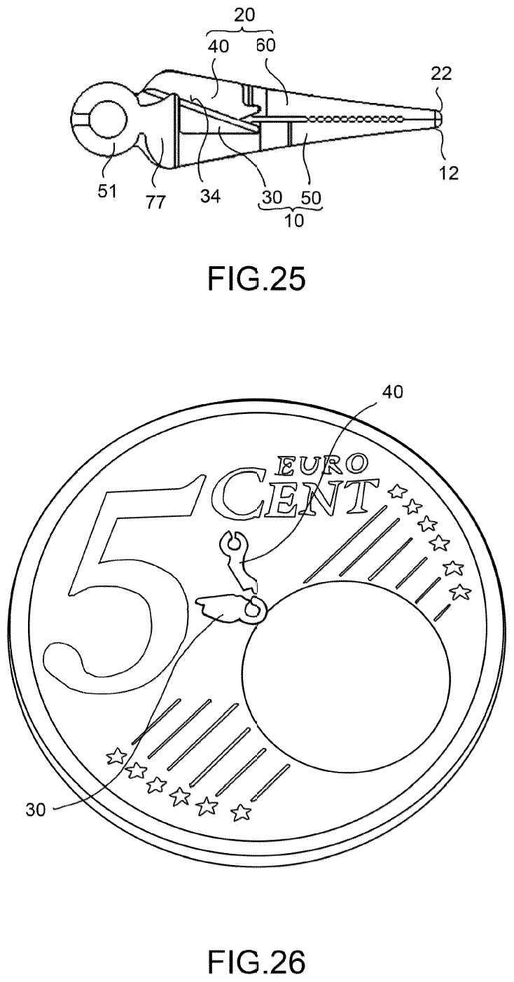

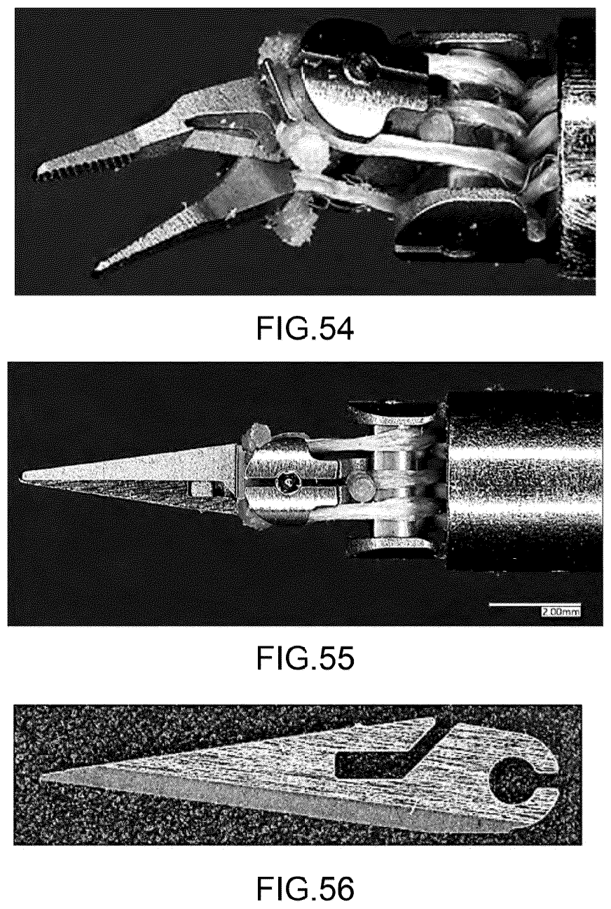

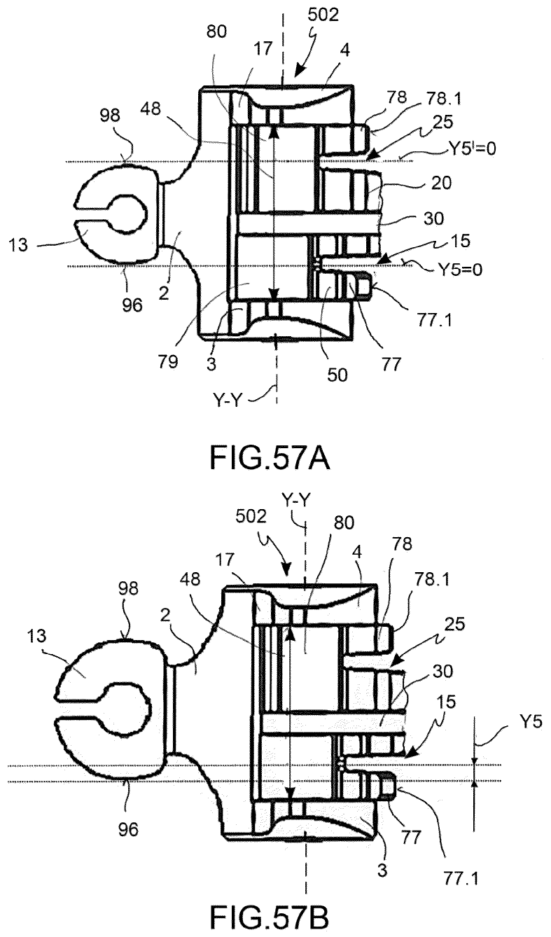

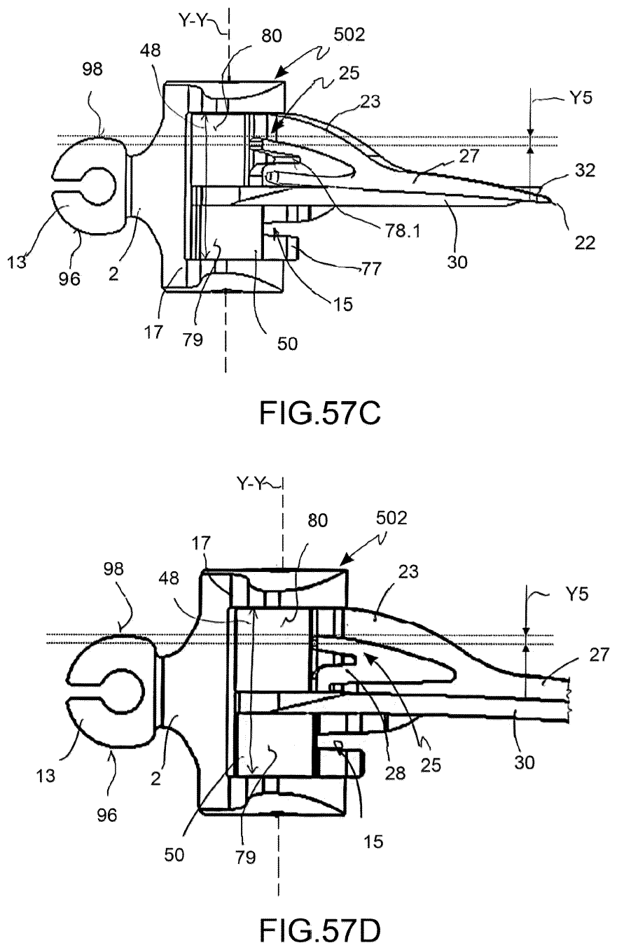

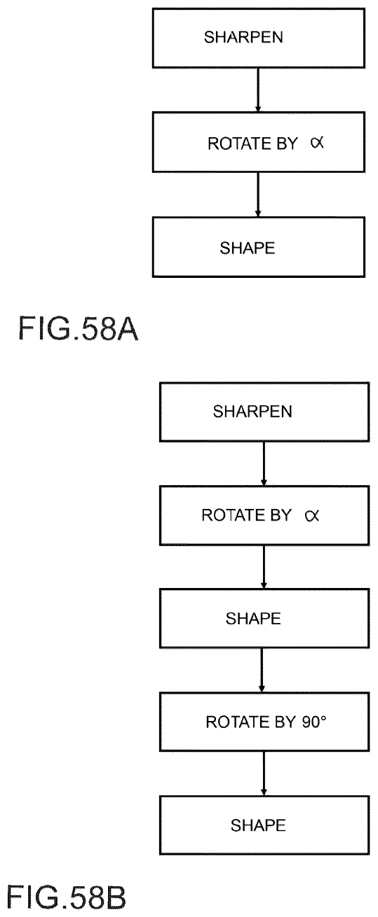

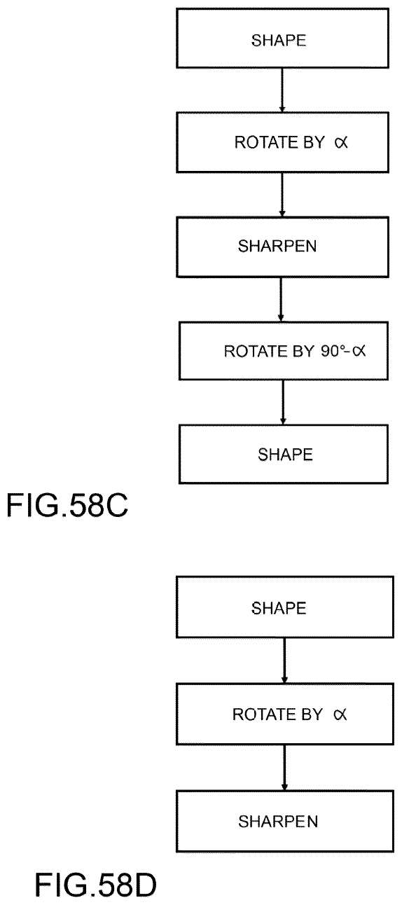

With reference to the previous description of embodiments of the invention, said surgical instrument 1 can be a surgical instrument of the needle-driver/sutures-cutter type (or “needle-holder/cutter” according to a commonly adopted terminology) as shown in FIGS. 4-30 and 54, for example. Embodiments of said surgical instrument 1 will be described below, where said surgical instrument 1 is a surgical instrument of the needle-driver/sutures-cutter type.

In accordance with an embodiment, the first free end 12 of the first tip 10 does not coincide with the distal end 32 of the blade portion 14, although the first free end 12 of the first tip 10 and the distal end 32 of the blade portion 14 can in accordance with an embodiment be made in a single piece in which the distal end 32 of the blade portion 14 is a longitudinally backward free end, i.e., more proximal to the first free end 12 of the first tip 10, as shown in FIG. 28, for example.

In accordance with an embodiment, said first tip 10 is made of two pieces or two links integral in rotation forming a blade link 30 and a blade holder link 50. In particular, the body of the blade holder link 50 comprises in a single piece a proximal attachment root 51 of the blade holder link 50, said first free distal end 12 and a first gripping surface 13 therebetween, and the body of the blade link 30 comprises said blade portion 14 with the cutting edge 34 thereof, in which the blade portion 14 of the blade link 30 comprises a distal end 32 which preferably acts as a drag engagement portion 37 and thus is not a free end when the blade link 30 is assembled to the blade holder link 50.

In accordance with an embodiment, the body of the second tip 20 comprises in a single piece said second distal free end 22 and a second gripping surface 63 between said second attachment root 21 and said second free end 22. It is possible to define a connecting portion 55, 65 for each tip 10, 20 between the attachment root 11 or 21 and the respective gripping surface 13, 63. When in use, the first gripping surface 13 of the first tip link 10 and the second gripping surface 63 of the second tip link 20 are intended to be mutually opposite and facing each other in rotation, to move in mutual contact to exert a gripping action for example on a surgical needle. Each gripping surface 13, 63 can be machined according to known techniques, forming reliefs and recesses to increase the gripping capacity.

In accordance with an embodiment, the body of the blade holder link 50 and the body of the second tip 20 each have a longitudinally elongated conformation extending from the respective attachment root to the respective free end, in which the respective gripping surface is placed close to the respective free end, and in which the roots of the blade holder link 50, the blade link 30 and the second tip 20 are next to one another, while in a respective connecting portion 55, 65 the body of the blade holder link 50 and the body of the second 20, which is longitudinally interposed between the respective root and the respective gripping surface 13, 63, an axial and longitudinal seat is obtained to receive the blade portion 14 of the body of the blade link 30 with the cutting edge 34 thereof. In other words, the elongated body of the blade holder link 50 and that of the second tip 20 are next to each other at the respective root and at the respective connecting portion 55, 65, and overlap each other at the respective gripping surface 13, 63, while the blade link 30 is next to the roots of the blade holder link 50 and the second tip 20 at the root 31 thereof and is next to and between the connecting portions of the blade holder link 50 and the second tip 20.

In accordance with an embodiment, the root of the blade link 31 is interposed between the roots of the blade holder link 50 and the second tip 20. Preferably, the blade link body 30 is also longitudinally elongated and comprises a blade link end 32, but is made shorter than the blade link body 50 and the second tip 20, and substantially extends in the longitudinal direction from the attachment roots, next to each other, to the gripping surface area 13, 63 of the blade holder link 50 and the second tip 20, i.e., the distal end 32 of the blade link 30 extends longitudinally to a level which is close to the proximal edge of the gripping surfaces 13, 63.

The gripping surfaces 13, 63 preferably act as closing stroke ends for the degree of freedom of opening/closing.

In accordance with an embodiment, the blade holder link 50 of the first tip 10 comprises a surface 18 facing axially inwards which is inclined away from the body of the blade link 30 axially internally delimiting an axial deformation recess 44 (or deformation seat 44) adapted to accommodate the blade portion 14 of the body of the blade link 30 when elastically bent by the action of the protruding surface of the counter-blade 24 during the cutting action. Therefore, the counter-blade portion 24 and the surface 18 facing axially inwards are both facing the blade portion 14 of the blade link 30 and both contacting thereto during the cutting action. The surface 18 facing axially inwards preferably belongs to said connecting portion 55 of the elongated body of the blade holder link 50. Preferably, the surface 18 facing axially inwards of the first tip link 10 serves as the axial stroke end abutment surface for the deformation of the blade portion 14 of the blade link 30 when deformed by bending by the counter-blade portion 24, during the cutting action. The profiles of the protruding surface of the counter-blade 24 and the axially facing surface 18 of the blade holder link 50 can be parallel to each other, and in an embodiment are correspondingly identical.

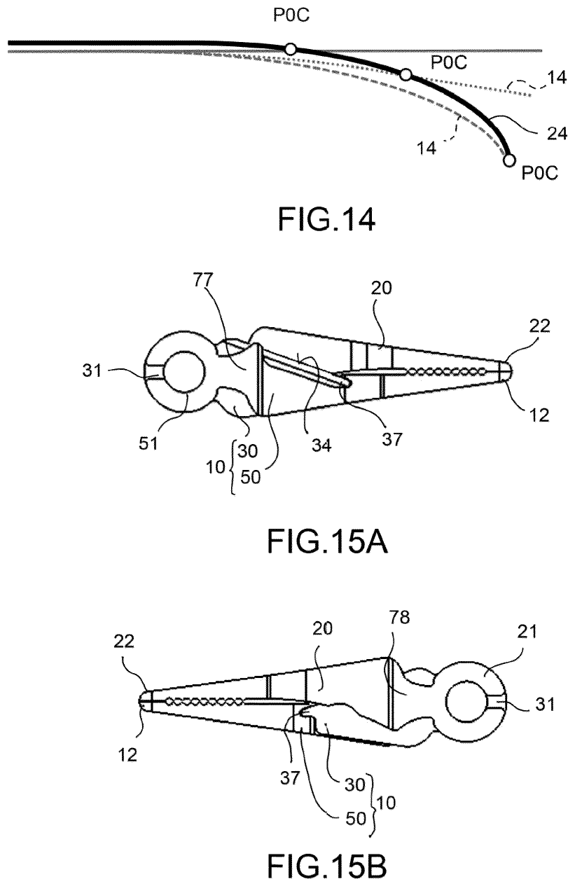

The at least one point of contact POC between the cutting edge 34 and the counter-blade portion 24 preferably varies in position and/or size as a function of the opening angle of the degree of freedom of opening/closing G (grip G), as diagrammatically shown for example in FIG. 14. In particular, at relatively high opening angles (e.g., angle in the range of)20°-30° the contact occurs in a portion which is more proximal to the cutting edge 34, i.e., closer to the attachment root 31 of the blade link 30, and as the opening angle reduces the contact moves in the distal direction, accentuating the elastic deformation bending of the blade portion 14 of the blade link 30 with respect to the root 31 of the blade link 30. Therefore, the deformed configuration of the blade link 30 when the first tip 10 and the second tip 20 are in a substantially closed configuration is maximally bent, and in any case more bent than the deformed configuration of the blade link 30 when the first tip 10 and the second tip 20 are in a partially closed and a partially open configuration. Preferably, when the opening angle is maximally open and the blade is free, the blade is straight the blade link has a substantially planar configuration.

In accordance with an embodiment, the counter-blade portion 24 can at least partially overlap the rotational approaching footprint of the body of the blade holder link 50 and the blade portion 14 of the blade link 30, when in an elastically deformed configuration, locally translates with respect to the rotational footprint of the blade holder link 50 in a direction transverse to the longitudinal extension direction of the blade holder link 50, i.e., in an external axial direction, although in accordance with a preferred embodiment, the counter-blade portion 24 and the surface 18 facing axially inwards of the blade holder link 50 are geometrically shaped so as not to overlap in their respective rotational clearances.

In accordance with an embodiment as shown in FIG. 28, for example, the root 31 of the blade link 30 is interposed between and a direct and intimate contact with the first prong 3 of the support structure and the root 51 of the blade holder link 50. The provision of a transverse bridge 33 in the body of the blade link 30 which crosses the rotational approaching footprint of the body of the counter-blade holder link brings the blade portion 14 with the cutting edge 34 thereof into contact with the counter-blade portion 24, i.e., between the blade holder link 50 and the second tip 20. In other words, the transverse bridge 33 can cross the connecting portion 55 of the elongated body of the blade holder link 50 and/or the root 51 of the blade holder link 50. In such a case, therefore, said first external contact surface 81 of the first tip 10 belongs to the root 31 of the blade link 30 and is in contact with the first internal surface 87 of the first prong 3, and an opposite contact surface facing axially inwards of the root 31 of the blade link 31 is in contact with a contact surface facing axially outwards of the root 51 of the blade holder link 50, and in which a said first internal contact surface 83 facing axially inwards of the first tip 10 belongs to the root 51 of the blade holder link 50 and is in contact with said opposite second internal contact surface 84 facing axially inwards of the second root 21 of the second tip 20. In accordance with this embodiment, then, the blade portion 14 with the cutting edge 34 remains interposed between the connecting portion 55 of the blade holder link body 50 and the connecting portion 65 of the second tip body 20, while the third root 31 of the blade link 30 is interposed between the first prong 3 of the support structure and root 51 of the blade holder link 50.

In accordance with an embodiment, the second tip 20 is made in two pieces, i.e., two links 40, 60 integral in rotation with each other, and in particular a counter-blade link 40 and a counter-blade holder link 60. In this embodiment, the counter-blade portion 24 is made in a single piece with said counter-blade link 40, i.e., the counter-blade link 40 comprises a proximal attachment root 41 of the counter-blade link 40 and the counter-blade holder link 60 comprises in a single piece a proximal attachment root 61 of the counter-blade holder link 60, said second gripping surface 63 and said second distal free end 22, in which the root 61 of the counter-blade holder link 60 and the root 41 of the counter-blade link 40 are next to and in direct and intimate contact with each other, jointly forming the second root 21 of the second tip 20. Where the second tip 20 is made in said two links 40, 60 mutually integral in rotation with each other, then the assembly formed by said root 51 of the blade holder link 50, and said root 31 of the blade link 30, and said root 41 of the counter-blade link 40 and said root 61 of the counter-blade holder link 60 is generally interposed between said two prongs 3, 4 of the support structure and in direct and intimate contact therewith.

By virtue of such a pack arrangement of the roots, impingements of the root 31 of the blade link 30 and of the root 41 of the counter-blade link 40, which are preferably thinner, with respect to the articulation pin 5 are avoided so as to provide a satisfactory certainty of positioning of the cutting edge 34 with respect to the counter-blade portion 24 for each opening angle of the degree of freedom of opening/closing G, thus providing extreme cutting precision.

Therefore, the root 61 of the blade holder link 60 preferably comprises an axially facing contact surface 89.1 and the root 41 of the blade holder link 40 comprises an axially facing contact surface 89.2, said contact surfaces 89.1, 89.2 are in direct and intimate contact with each other, and preferably are parallel to each other and parallel to the other contact surfaces 81, 82, 83, 84, 85, 86, 87, 88 of the roots and the prongs, and even more preferably extend in a plane orthogonal to the common rotation axis Y-Y.

In accordance with an embodiment in which a counter-blade portion 24 is provided, which is made on a separate counter-blade link 40 having a proximal attachment root 41, then the root 31 of the blade link 30 is axially interposed between said root 41 of the counter-blade link 40 and the root 51 of the blade holder link 50, and in direct and intimate contact therewith, and in which said root 41 of the counter-blade link 40 is axially interposed between said root 30 of the blade link 30 and said root 61 of the blade holder link 60, and in direct and intimate contact therewith, to provide a reaction to the elastic bending of the blade portion 14 during the cutting action.

As mentioned above, the roots preferably have a cylindrical geometry about the common rotation axis Y-Y, and where the root 41 of the counter-blade link 40 has a significantly smaller thickness that the root 51 of the blade holder link 50 and the root 61 of the blade holder link 60, said root 41 of the counter-blade link 40 has a cylindrical geometry of the discoid type, similar to the root 31 of the blade link 30.

Where the second root 21 of the second tip 20 is formed by said root 41 of the counter-blade link 40 and said root 61 of the counter-blade link 60, each of said roots 41 and 61 will be provided with a second through hole 26, according to any one of the embodiments described above. In such a case, the second through hole 26 of the root 61 of the blade holder link 60 and the second through hole 26 of the root 41 of the blade holder link 60 can be circular holes coaxial with each other and can have the same diameter. In accordance with an embodiment, said second through hole 26 of the root 41 of the counter-blade link 40 has a hole edge in direct and intimate contact with the articulation pin 5 for the entire extension of the hole edge, to exert with an arc surface thereof the thickness of the hole edge a reaction to the friction exchanged between the blade link 30 and the counter-blade portion 24 of the counter-blade link 40 during the cutting action.

In accordance with an embodiment in which the blade link 30 and the blade holder link 50 further comprise respective drag engagement portions 37, 57 to make the blade link 30 and the blade holder link 50 integral in rotation, the drag engagement portion 57 of the blade holder link 50 is made as a drag seat 57 delimited by the connecting portion 55 of the blade holder link body 50 and by a drag tooth 57.0 forming a seat 57 as an undercut with respect to the first gripping surface 13, i.e., a seat 57 which opens proximally and also extends axially, to receive the distal end 32 of the blade link 30 in rotation drag contact while receiving the deformation of the distal end 32 of the blade link 30 in the axial direction. In other words, a portion close to or at the distal end 32 of the blade link 30 serves in this embodiment as a drag engagement portion 37 of the blade link 30 which is received in rotation drag contact, i.e., in the opening/closing direction, inside the drag seat 57 of the blade holder link 50, and at the same time the distal end 32 of the blade link 30 is free to deform axially externally inside the same drag seat 57 which therefore forms part of the axial deformation seat 44 for the blade portion 14. In other words, the drag seat 57 extends distally with respect to the surface 18 facing axially inwards of the first tip link 10, i.e., with respect to the surface 18 which can act as an axial abutment for the bending of the blade portion 14. In such a case, the drag seat 57 has an axial extension such as to accommodate the distal end 32 of the blade link 30, thus receiving together with said deformation seat 44 the deformation of the blade link 30 during the cutting action. The distal end 32 of the blade link 30 can comprise a distal portion of said cutting edge 34, and in such a case said distal portion of said cutting edge 34 acts as a drag counter-surface in the opening direction 37.2 cooperating against a respective opening drag surface 57.2 of the drag tooth 57.0 delimiting the drag seat 57 of the blade holder link 50.

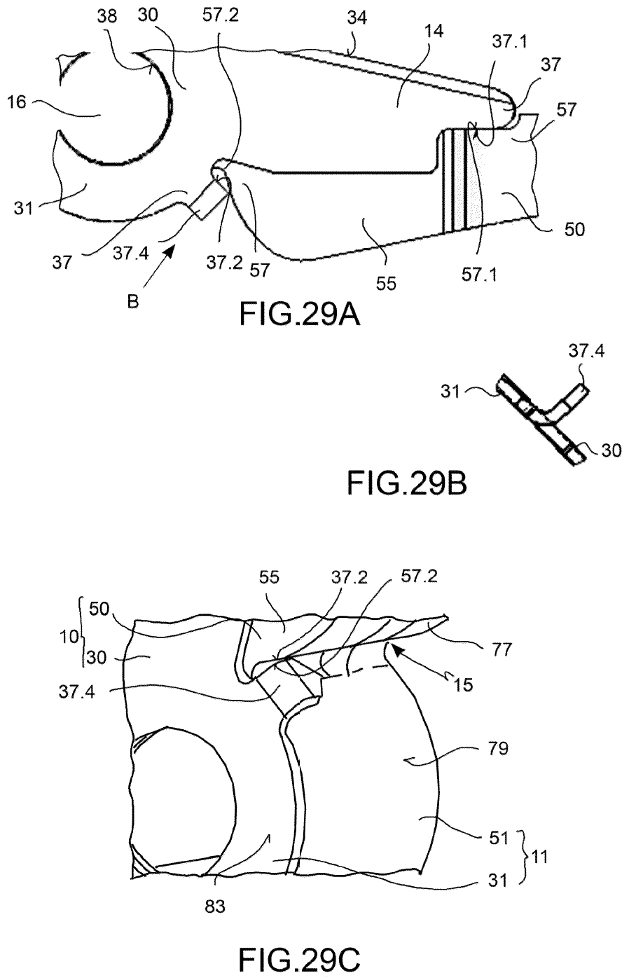

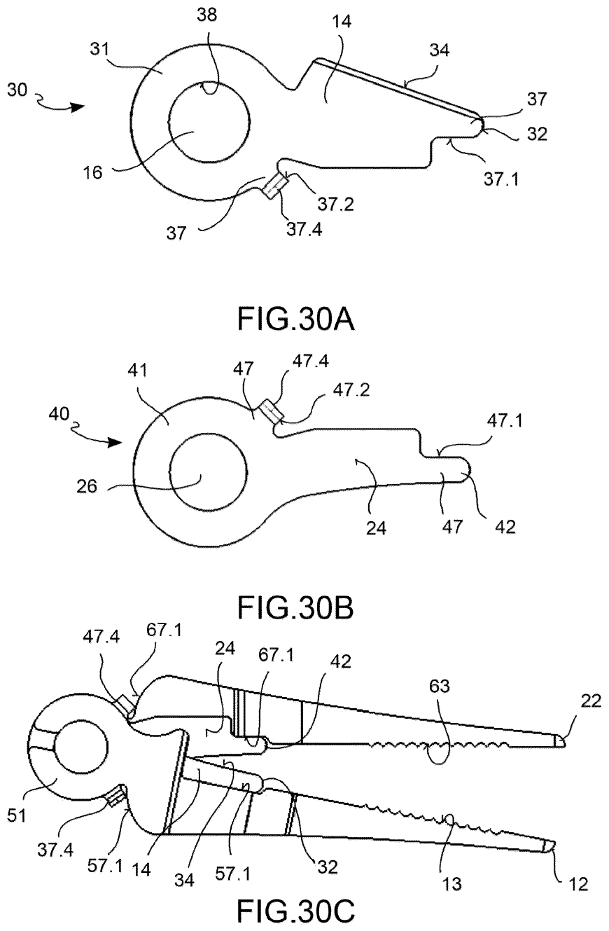

In accordance with an embodiment in which the blade link 30 and the blade holder link 50 further comprise respective drag engagement portions 37, 57 to make the blade link 30 and the blade holder link 50 integral in rotation, the drag engagement portion 57 of the blade holder link 50 is made as two distinct and separate drag surfaces. In other words, the opening drag surface 57.2 and the closing drag surface 57.1 of the blade holder link 50 can be placed at different distances from the common rotation axis Y-Y, as well as the opening drag surface 37.2 and the closing drag surface 37.1 of the blade link 30 can be arranged at different distances from the common rotation axis Y-Y, for example on different protrusions of the blade link 30, as shown in FIG. 29A, for example. In particular, with reference to FIGS. 29A, 29B and 29C, as well as FIG. 30A, the root 31 of the blade link 30 can comprise a radial drag ear 37.4 folded onto the first root 11 of the first tip link 10, said drag ear 37.4 comprising said opening drag surface 37.2.

In accordance with an embodiment, said first tip link 10 and said blade link 30, being made in separate pieces, are integral in rotation with each other in a releasable manner and the release can preferably occur only by disassembling the articulated end-effector 9.

In accordance with an embodiment, the second tip 20 comprises a thread-stop wall 48 facing the common rotation axis Y-Y delimiting a thread-stop recess 48.1 for receiving a suture thread 68 to keep the suture thread 68 in contact with the cutting edge 34 of the blade of the blade link 30 during a cutting closure. The provision of the thread-stop wall 48 prevents the suture thread 68 from sliding distally during the cutting action beyond the distal end 32 of the blade, as an effect of the closing action.

The thread-stop wall 48 and the thread-stop recess 48.1 preferably face the gripping side P2 of the second tip 20, for example the thread-stop wall 48 is an arched wall which has a concavity defining the recess 48.1 facing the cutting side P2 of the second tip 20. The recess 48.1 can be made in the form of a notch provided in the body of the second tip 20 and in such a case the thread-stop wall 48 is a wall delimiting said notch. The recess 48.1 can be made in the form of an undercut wall provided on a protrusion of the body of the second tip 20 and in such a case the thread-stop wall 48 is an undercut wall of said protrusion facing the common rotation axis Y-Y.

In accordance with an embodiment, the thread-stop wall 48 delimits with an axially internal edge thereof the counter-blade portion 24 from the cutting side P2 of the second tip 20. Where the counter-blade surface 24 is made in a separate piece with respect to the second tip link 20, the thread-stop wall 48 and the recess 48.1 can be formed in the body of the counter-blade link 40.

In accordance with an embodiment, the blade holder link 60 of the second tip 20 comprises an axial recess 45 forming a housing seat 45 for the blade holder link 40. Said axial recess 45 is preferably axially delimited by a surface 43 facing axially inwards of the counter-blade holder link 60.

In accordance with a preferred embodiment, the counter-blade link 40 is elastically deformable by bending. Thereby, when the cutting edge 34 of the blade link 30 is in mechanical interference contact with the counter-blade portion 24 of the counter-blade link 40 to exert a cutting action, the body of the counter-blade link 40 elastically bends in the axial direction as well.

The counter-blade link 40 is preferably made from an elastic sheet or strip and is pre-curved to form a curved, protruding counter-blade portion 24 having a concavity facing axially inwards, in order to elastically bend the blade link 30 during the cutting action. The provision of a counter-blade link 40 having a curved, protruding counter-blade portion 24 elastically deformable by bending allows obtaining an elastic reaction between the surface 68 facing axially inwards of the axial recess 45 of the counter-blade holder link 60 and the cutting edge 34 of the blade link 30, during the cutting action. In particular, the counter-blade link 40 comprises a resting surface 46 directed axially and opposite to the counter-blade portion 24 which abuts against said surface 68 facing axially inwards of the axial recess 45 of the counter-blade holder link 60 to allow the counter-blade link 40 to provide an elastic action on the cutting edge 34 of the blade link 30 aimed at resiliently bending the blade link 30 during the cutting action. For example, the counter-blade link 40, where present, can be made of spring steel.

The counter-blade link 40 can have at least some, but also all, of the features and 2.5 properties described above with reference to the blade link 30. The thickness of the counter-blade link 40 can be substantially comparable to or equal to the thickness of the blade link 30, as described above. In accordance with an embodiment, the counter-blade link 40 comprises a counter-blade cutting edge 64 which is preferably arranged opposite to the cutting edge 34 of the blade link 30, i.e., in other words the cutting edge of the counter-blade 64 faces the cutting side P2 of the second tip 20. The proximal attachment root 41 of the counter-blade link 40 can have at least some, but also all, of the features and properties described above with reference to the root 31 of the blade link 30. The root 41 of the counter-blade link 40 can comprise a radial cutting channel 49 misaligned with the radial cutting channel 39 of the blade link 30 to prevent the edges of the cutting channels 39, 49 from engaging during the opening/closing action.

In accordance with an embodiment, to make the counter-blade link 40 and the counter-blade holder link 60 integral in rotation, a drag engagement is provided along the longitudinal extension of the counter-blade surface 24 or distally with respect thereto. Preferably, the drag engagement is obtained close to or at the distal end 42 of the counter-blade link 24. In accordance with an embodiment, the blade holder link 60 comprises a drag seat 67 having an opening drag surface 67.2 and an opposite closing drag surface 67.1 to make the blade holder link 40 integral in rotation. The drag seat 67 can be placed distally in a drag seat made as an undercut with respect to the second gripping surface 63 of the counter-blade holder link 60 to receive the distal end 42 of the counter-blade link 40. In accordance with an embodiment, said distal end 42 of the counter-blade link 40 comprises an opening drag surface 47.2 in dragging contact with said opening drag surface 67.2 of the counter-blade holder link 60, and an opposite closing drag surface 47.1 in dragging contact with said closing drag surface 67.1.

In accordance with an embodiment as shown in FIG. 29B, for example, the counter-blade link 40 comprises a radial drag ear 47.4 folded on the root 61 of the counter-blade link 60, said drag ear 47.4 of the counter-blade link 40 comprising an opening drag surface 47.2 in drag contact with an opening drag surface 67.2 which is for example placed on a back portion D2 of the connecting portion 65 of the body of the counter-blade link 60, and in which the counter-blade link 40 further comprises a closing drag surface 47.1 placed close to the distal end 42 of the counter-blade link 40 in drag contact with a closing drag surface 67.1 of the counter-blade link 60.

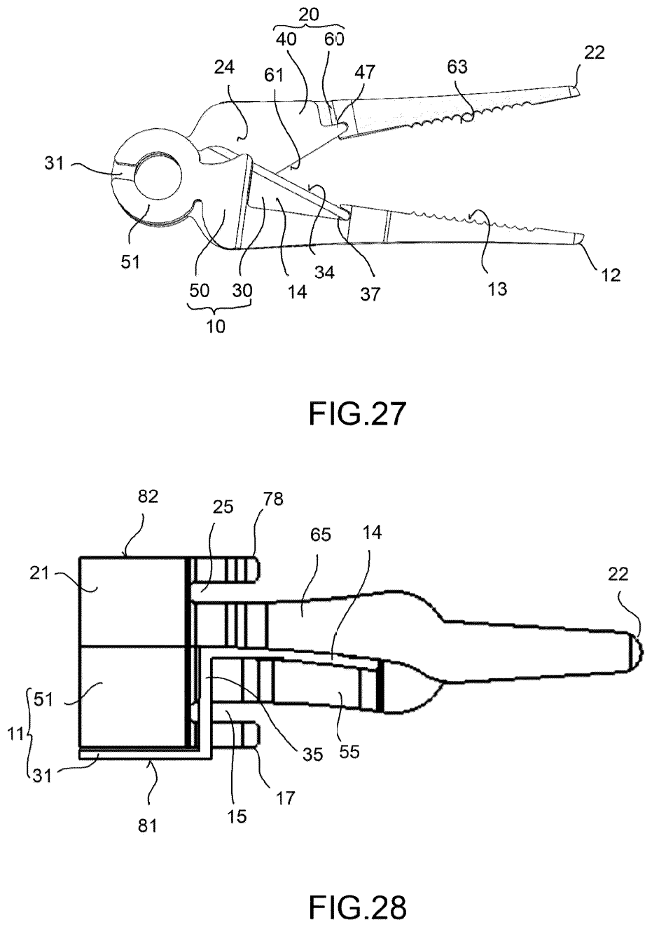

In accordance with an embodiment as shown in FIG. 27, for example, the counter-blade cutting edge 64 can have a concave shape with respect to the opening/closing direction.

A cutting method for a surgical instrument will be described below.

Such a cutting method is adapted to be performed with a surgical instrument 1 according to any one of the embodiments described above.

In accordance with an embodiment, cutting for a surgical instrument comprises the steps below.

The method comprises providing an articulated end-effector 9 at the distal end of a rod 7 comprising a support structure, a blade portion 14 having a cutting edge 34, and a counter-blade portion 24 forming a distal rotational joint 502.

The articulated end-effector can comprise a link 90 and the support structure can belong to a support link 2 articulated to the connection link 90 in a proximal rotational joint 509.

The method comprises longitudinally sliding the actuation tendons 71, 72; 75, 76 of at least one pair of antagonistic tendons on one or more convex ruled surfaces 97, 99; 96, 98 with parallel generatrices of the support structure, to orient the cutting edge 34 of the blade link 30 in a desired orientation. In accordance with an embodiment, this step includes longitudinally sliding the actuation 3.5 tendons 71, 72; 75, 76 of at least one pair of antagonistic tendons on one or more convex ruled surfaces 97, 99; 96, 98 with parallel generatrices at least one of a connection link 90 and a support link 2.

The method comprises longitudinally sliding the actuation tendons 71, 72; 73, 74 of at least one pair of antagonistic actuation tendons of the distal rotational joint 502 on one or more convex ruled surfaces 97, 99; 96, 98 with parallel generatrices of the connection link 90 and the support structure, for example said support link 2, to bring the cutting edge 34 into contact with said counter-blade portion 24.

The method comprises elastically bending at least one of the cutting edge 34 and the counter-blade portion 24, making a mechanical interference contact therebetween, exerting a cutting action.

The step of longitudinally sliding the antagonistic tendons 71, 72; 73, 74 of at least one pair of antagonistic actuation tendons of the distal rotational joint 502 on the convex ruled surfaces 97, 99; 96, 98 with parallel generatrices of the connection link 90 and the support link 2, can comprise the step of winding at least one movement tendon 71, 72; 73, 74 of the distal rotational joint 502 on the convex ruled surfaces on which it slides, by a winding angle between 60° and 300°, and preferably greater than 120°.

In accordance with a general embodiment, a robotic surgery system 101 is provided, comprising at least one surgical instrument 1 according to any one of the embodiments described above. The robotic surgery system 101 is thus capable of performing surgical or microsurgical procedures including cutting a biological tissue and/or cutting sutures.

In accordance with an embodiment, said robotic surgery system 101 comprises at least two surgical instruments, at least one of which is a surgical instrument 1 according to any one of the embodiments described above and the other surgical instrument can be a surgical instrument of the needle-driver type or a surgical instrument of the dilator type, although in accordance with an embodiment both surgical instruments are surgical instruments 1 according to any one of the embodiments described above, not necessarily mutually identical although they can be. For example, a surgical instrument of the at least two surgical instruments can be a surgical instrument of the surgical scissor type and another surgical instrument of the at least two surgical instruments can be a surgical instrument of the needle-driver/scissor type.

The robotic surgery system 101 preferably comprises at least one robotic manipulator 103 and the at least one surgical instrument 1 is operatively connected to said at least one robotic manipulator 103. For example, a sterile surgical barrier (not shown), such as a sterile surgical cloth, for example, is interposed between the at least one robotic manipulator 103 and the backend portion 104 of the at least one surgical instrument 1. The robotic manipulator 103 can comprise motorized actuators for stressing said actuation tendons of the degrees of freedom of pitch P, yaw Y and grip G. i.e., cutting G of the surgical instrument 1, and a motorized actuator for rotating the surgical instrument 1 about the shaft 7 defining a degree of freedom of roll R. The robotic surgery system 101 can comprise a support portion 106 (“cart” or tower) for example comprising wheels or other ground contact units, and an articulated positioning arm 105, for example manually movable i.e., passive, extending between the support portion 106 and the at least one robotic manipulator 103. In accordance with an embodiment, the robotic surgery system 101 comprises at least one master console 107 for controlling the at least one surgical instrument 1 and preferably also the respective robotic manipulator 103 according to a master-slave architecture, and preferably the robotic surgery system 101 further comprises a control unit operatively connected to the master console 107 and the robotic manipulator 103 for determining the tracking of the surgical instrument 1 to at least one master control device 108 of the master console 107. In accordance with an embodiment, the master console 107 comprises at least one master control device 108 which is unconstrained, i.e., mechanically disconnected from the ground, and a tracking system, for example optical and/or magnetic.

### Wire Electro-Erosion Manufacturing

A wire electro-erosion manufacturing method will be described below, which achieves the sharpening of the cutting edge of a blade portion 14.

In accordance with a general embodiment, a method of manufacturing one or more blades by wire electro-erosion comprises the steps of: providing a wire electro-erosion machine 200 having a cutting wire 202 and providing a fixture 214 mounted to the wire electro-erosion machine and mounting at least one workpiece 204 to the fixture 214.

The method further comprises the step of sharpening at least one edge to be sharpened 234 of the at least one workpiece 204 by performing a sharpening through cut with the cutting wire 202 on the at least one workpiece 204.

The sharpening step achieves a sharpening process for obtaining said cutting edge 34 of the blade portion 14 of the articulated end-effector 9. In the following description, aspects of the sharpening step that which also applicable in the context of this method will be explained in detail, unless otherwise specified.

A method of manufacturing one or more blades by wire electro-erosion will be described below.

In accordance with a general embodiment, a method of manufacturing one or more blades is provided. Such one or more blades are preferably intended to form miniaturized cutting elements.

In accordance with an embodiment, a blade of said one or more blades manufactured by the method forms a blade portion 14 according to any one of the embodiments described above. In accordance with an embodiment, a blade of said one or more blades manufactured by the method forms a blade link 30 In accordance with any one of the embodiments described above. In accordance with an embodiment, a blade of said one or more blades manufactured by the method forms a counter-blade link 40 In accordance with any one of the embodiments described above.

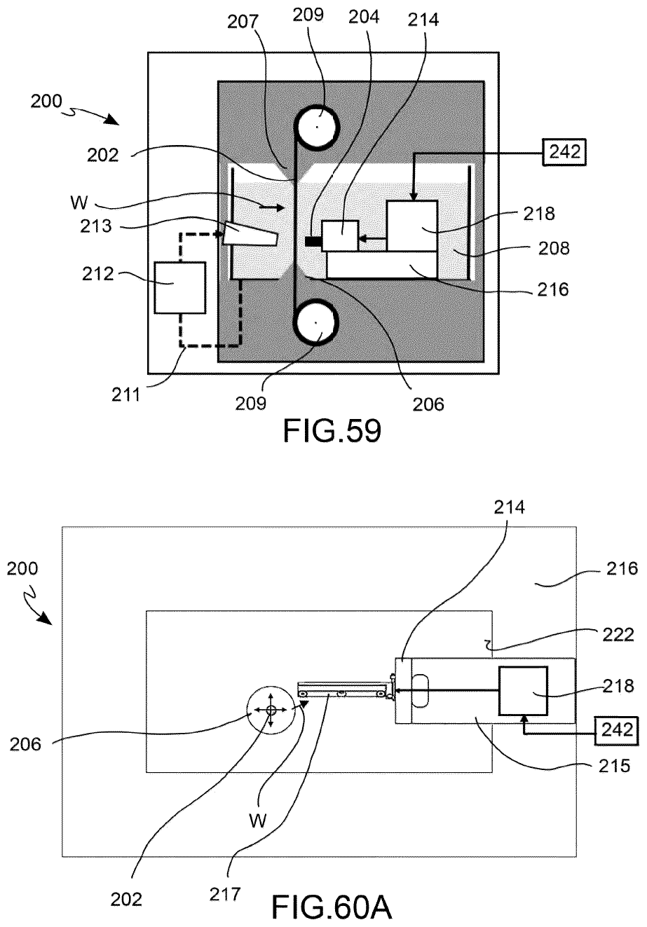

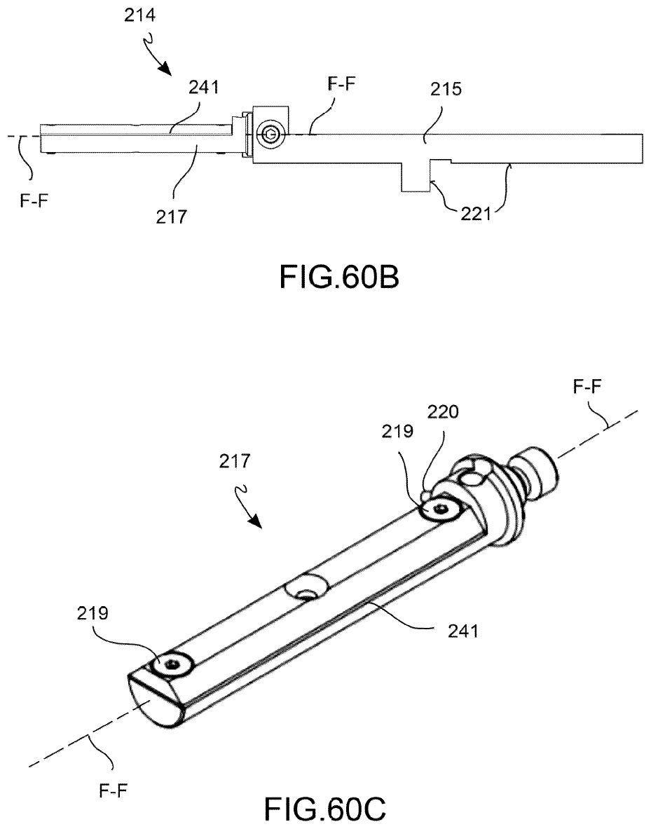

The method comprises the step of providing a wire electro-erosion machine 200 comprising a cutting wire 202, as shown in FIG. 59, for example. The cutting wire 202 preferably extends longitudinally between two heads 206, 207 of the wire electro-erosion machine 200 when in operating conditions. To perform the cut (i.e., electro-erosion), the cutting wire 202 advances along a cutting path in a feeding direction W (or cutting direction W) which is substantially orthogonal to the longitudinal extension of the cutting wire 202, i.e., the feed direction is substantially orthogonal to the sliding direction of the portion of the cutting wire 202 between the two heads 206, 207 of the machine 200, in a manner known per se. Each of the two heads 206, 207 can be associated with a reel 209 or winding/unwinding roller 209 for the cutting wire 202. When in operating condition, the cutting wire 202 runs winding on one reel as it unwinds from the other reel, and the heads 206, 207 guide the cutting wire 202 in the feeding direction W (or cutting direction W) to perform a cut on the workpiece.

The wire electro-erosion machine 200 preferably comprises a tank 208 to be filled with dielectric liquid inside which the electro-erosion of at least one workpiece 204 occurs when in operating conditions. The electro-erosion machine 200 can further comprise a hydraulic circuit comprising a hydraulic duct 211 fitted with a pump 212 and a filter which withdraws and filters dielectric fluid from the tank 208 and ending with a nozzle 213 which directs dielectric fluid onto the workpiece 204.

The at least one workpiece 204 is preferably made of electrically conductive material, such as metal, or is coated with electrically conductive material.

The wire electro-erosion machine 200 further comprises at least one jig 214 or fixture 214 which is rotatable with respect to the cutting wire 202 (i.e., with respect to the cutting section of the cutting wire 202) about a rotation axis F-F which is transverse, and preferably orthogonal, to the longitudinal extension of the cutting wire 202. For example, the rotation axis F-F of the jig 214 extends substantially horizontally while the cutting portion of the cutting wire 202 substantially vertically.

The method comprises the step of mounting at least one workpiece 204 on the jig 214, for example by fixing the workpiece 204 by fixing screws or other fasteners to the jig 214 such that the at least one workpiece 204 is integral in rotation with a portion of the jig 214. Thereby, rotating the jig 214 about the rotation axis F-F thereof results in a rotation of the workpiece 204 with respect to the cutting wire 202.

The jig 214 can comprise a fixing portion 215 fixed to a bracket of the worktop 216 inside the tank 208 of the wire electro-erosion machine 200, and a housing portion 217 receiving said at least one workpiece 204 for example in at least one of the housing seats 241 thereof, in which the housing portion 217 of the jig 217 is rotatable with respect to the fixing portion 216 to the machine 200 about said rotation axis F-F. In accordance with an embodiment, the fixing portion 216 to the machine 200 of the jig 214 comprises positioning rectified surfaces 221 intended to abut against rectified counter-surfaces 222 of the bracket of the worktop 216 of the machine 200.

The housing portion 217 of the jig 214 can have an elongated body extending along the rotation axis F-F and can be pivotally connected to the fixing portion 215. Rotating only the housing portion 217 with respect to the fixing portion 215 allows minimizing the translation movements of the workpiece 204 with respect to the lower head 206 of the machine which can derive from the rotation step, as it is generally desirable to position the workpiece 204 close to the lower head 206 during cutting to minimize the deformability of the cutting wire 202. In other words, a rotation of the jig could move the workpiece with respect to the cutting wire in the longitudinal extension direction of the cutting wire between the machine heads, for example bringing the workpiece located close to a head located at the median zone of the section of the cutting wire extended between the heads of the machine, which is more deformable transversely with respect to the section close to one of the heads with consequent variation of the cutting features, for example in terms of finish and/or cutting resolution. Typically, in fact, a wire electro-erosion machine is adapted to perform a better and more precise cutting machining when the workpiece is arranged close to at least one of the heads where the cutting wire is less transversely deformable while sliding longitudinally, as well as when the heads are close to each other thus shortening the longitudinal extension of the portion of the cutting wire extending between the machine heads to limit the transverse movements thereof when in operating conditions, i.e., cutting, as well as when the sliding direction of the wire is perfectly orthogonal to the plane identified by the feeding direction W or cutting direction W. The electro-erosion machine 200 can be provided with the functionality which includes crossing the heads 206, 207, i.e., translating the heads so as to incline the cutting wire 202 with respect to the workpiece 204, but in light of the above, in order to obtain a satisfactory cutting accuracy, the heads must be kept close and therefore such a functionality of crossing the heads allows inclining the cutting wire with respect to the workpiece at a maximum of an angle around 5°, in general terms, which makes this solution of crossing the heads of the wire electro-erosion machine unsuitable for obtaining a sharpening.

The housing seat 241 of the housing portion 217 of the jig 216 can be formed by a longitudinal slot 241 along the body of the housing portion 217 for receiving a workpiece 204 which is a plate-like body, tightening it, for example by clamping and positioning elements 219, in a central portion thereof so that the plate-like body of the workpiece 204 forms two opposite cantilevered flaps 205 which can both be subject to wire electro-erosion machining. The workpiece 204 can be tightened in other manners. Positioning elements such as holes or notches can be provided on the body of the workpiece for mounting the workpiece to the jig 214.

Preferably, the extension of the cantilevered portion of each cantilevered flap 205 of the plate-like body of the workpiece 204 projecting cantilevered from the housing portion 217 of the jig 214 is chosen so as to minimize the vibrations which can arise during the action of the cutting wire 202 on the workpiece 204 as well as on the jig 214 and which would lead to cutting uncertainty. Screws or tightening screws can be provided as tightening and positioning elements 219 adapted to tighten the housing seat and meanwhile acting as positioning elements of the workpiece 204 in the seat. In accordance with a possible operating mode, one or more fixing and positioning elements 219 are designed to cross the body of the workpiece 204 for example in a through hole thereof in order to exert the fixing action thereof to the jig and positioning action with respect to the jig and the cutting edge.

In accordance with a possible operating mode, the workpiece 204 comprises a plate-like body having a thickness 210 in the range from 0.05 mm to 0.5 mm. The plate-like body can be obtained from a strip tape of material or from a full piece of sliced material. The plate-like body can be a deformable elastic body in bending.

The method comprises the step of sharpening at least one edge to be sharpened 234 of the at least one workpiece 204 by making at least one sharpening through cut with the cutting wire 202 on the at least one workpiece 204. The advancement of the cutting wire 202 along a sharpening cutting path makes a through cut on the at least one workpiece which determines the sharpening of at least one edge to be sharpened 234 of the workpiece 204 making the edge to be sharpened 234 a cutting edge 34.

The at least one edge sharpened by the method will form the cutting edge 34 of the blade portion 14 and/or the cutting edge 34 of the body of the one or more blade links 30.

The method further comprises the step of shaping the at least one workpiece 204 by performing at least one shaping through cut on the at least one workpiece 204 with the cutting wire 202. The advancement of the cutting wire 202 along a shaping cutting path 230 makes a through cut on the at least one workpiece 204 which determines the shaping of the one or more blades made by the manufacturing method. Not necessarily, the shaping step results in the separation of the single blade and for example a bridge 231 of a material can connect the blades together at the end of the shaping step. The shaping step can provide an end 32 on the workpiece which can form the distal end of the blade portion 14, for example of a blade link 30.

Of course, the sharpening and shaping steps can be performed in any order.

Between the sharpening step and the shaping step, the further step of rotating the jig 214 about the rotation axis F-F thereof by a sharpening rotation angle α is performed.

In accordance with an embodiment, a motor 218, for example an electric motor, is associated with the jig 214 to rotate the housing portion 217 of the jig 214 with respect to the fixing portion 215. In such a case, the step of rotating the jig 214 is performed by operating the motor 218. The electro-erosion machine 200 also preferably comprises at least one electronic control system 242 and the motor 218 is operatively connected to said electronic control system 242 of the machine 200. Therefore, it is possible to automate the step of rotating the jig 214.

The sharpening rotation angle α is different from 90°.

“Different from than 90°” is meant to indicate an angle significantly different from 90°, in which the deviation from 90° is at least 10°, i.e., the sharpening rotation angle α is different from 90°±10°. Preferably, it is meant to indicate a sharpening rotation angle α different from 90° in absolute value, i.e., in any rotation direction (clockwise or counterclockwise) about the rotation axis F-F.

The provision of a sharpening angle α other than 90° allows making an acute angle ß in the cross-section of the workpiece body, forming a cutting edge 34.

In accordance with a preferred embodiment, the sharpening angle α is an acute angle and net of the tolerance of +10° C. an be understood as an angle less than 80° in absolute value and preferably greater than 10°.

The sharpening angle α, which measures the rotation of the workpiece with respect to the cutting wire 202, can be chosen so as to achieve the desired cutting performance of the cutting edge 34 because the choice of the sharpening angle α determines the acute angle ß in the cross-section of the cutting edge 34.

By virtue of such a method, at least two through cuts can be obtained on the workpiece on two cutting planes which are not orthogonal to each other, in which at least one through cut is sharpened, i.e., it makes a cutting edge 34 and the other through cut is of shaping.

Where the workpiece has a plate-like body, preferably the shaping through cut is performed by orienting the cutting wire 202 substantially orthogonally with respect to the plane of the plate-like body, to make cutting walls in the thickness of the short and robust workpiece, while the shaping through cut is performed by orienting the cutting edge obliquely with respect to the plane of the plate-like body, making a sharp profile in the thickness, i.e., in the cross-section, of an edge of the workpiece.

The jig 214 can comprise mechanical stroke ends 220, for example two opposite stroke end ridges 220 facing opposite end stroke abutment surfaces, which are located on the housing portion 217 and on the fixing portion 215 of the jig 214. In such a case, the rotating step can comprise bringing the housing portion 217 of the jig 214 in abutment against a stroke end ridge 220 of the fixing portion 215 of the jig 214. The stroke ends 220 may be releasably associated with the jig 214 so as to allow the sharpening rotation angle α to be adjusted, and for example one or more stroke ends can be extractable and retractable.

The rotating step is performed, avoiding disassembling the workpiece 204 from the jig 214 as well as avoiding disassembling the jig 214 from the wire electro-erosion machine 200. Therefore, replacements are avoided. The rotation axis F-F of the jig 214 can extend through the body of the workpiece 204, for example it can extend along the thickness 210 of the workpiece 204 where the workpiece has a plate-like body (for example it is a strip, a ribbon, a plate, a sheet) and in such a case the rotation of the jig 214 can also result in a rotation of the plate-like body of the workpiece 204 about one of the axes thereof (for example: median axis, axis of symmetry).

By virtue of such a method, it is possible to manufacture one or more blades by making two through cuts on the workpiece 204 by wire electro-erosion on two cutting planes which are non-orthogonal to each other and rotated by said sharpening angle α, a through cut being of sharpening, while avoiding disassembling the workpiece 204 from the jig 214 as well as disassembling the jig 214 from the wire electro-erosion machine 200. Thereby, a high cutting accuracy of the sharpening and shaping cuts is achieved because the at least one workpiece is prevented from being repositioned with respect to the machine, and for example also the calibration of the electronic control system of the electro-erosion machine 200 is more reliable and can be performed only once, for example after the assembly step and before both the sharpening and shaping steps.

To perform the zeroing and calibration of the electro-erosion machine 200, the method can comprise the steps of: identifying reference point 229 and approaching said reference point 229 with the cutting wire 202, prior to the sharpening step. The reference point 229 can be identified by contacting one or more points of the workpiece 204 one or more times with the cutting wire 202. For example, two orthogonal sides of the plate-like body of the workpiece can be contacted to identify a reference point 229 which coincides with a vertex of the plate-like body of the workpiece 204. In accordance with an operating mode, said reference point 229 belongs to the edge to be sharpened 234 of the workpiece 204. Not necessarily, the approaching step causes the cutting wire 202 to reach the reference point 229. The cutting start point 232, 235 of the sharpening 240 and/or shaping 230 cutting path can be close to the reference point 229 or coincident with the reference point 229. In accordance with a possible operating mode, the cutting start point 232, 235 of the sharpening 240 and/or shaping 230 cutting path is placed in a position having a predefined geometric relationship with the reference point 229.

In accordance with a possible operating mode, the identification and approaching steps are performed before each of said sharpening and/or shaping steps.

In accordance with a possible operating mode, the identification and approaching steps come only once, before both the sharpening and shaping steps.

In accordance with a possible operating mode, the identification step comprises identifying a single point of origin of the cutting path which serves as the point of origin for both the sharpening cutting path and the shaping cutting path, and the approaching step comprises approaching said single point of origin with the cutting wire both in preparation for the sharpening step and in preparation for the shaping step. In accordance with an operating mode, prior to both the sharpening and shaping steps, the method comprises the step of identifying a single point of origin of the cutting path which serves as the point of origin for both the sharpening cutting path and the shaping cutting path, and approaching, preferably until reaching, said single point of origin with the cutting wire 202 both in preparation for the sharpening step and in preparation for the shaping step. Thereby it is possible to reset the machine, i.e., calibrate the machine only once at the beginning of the method, avoiding recalibration. The identification of said point of origin can be performed by contacting a known reference on said fixture 214 with the cutting wire 202. The identification of said point of origin can be performed by contacting a known reference on said workpiece 204 with the cutting wire 202.

In accordance with a possible operating mode, the method makes a plurality of blades on a single workpiece 204, and said sharpening step and said shaping step are the same for all the blades of said plurality. For example, a single sharpening trajectory 240 is provided, with starting point 235 and ending point 236 for multiple blades, whether they are the same or different.

In accordance with a possible operating mode, the sharpening step is performed by a single cutting sharpening trajectory 240 of the cutting wire 202 and said shaping step is performed by a single cutting shaping trajectory 230 of the cutting wire 202. Each cutting trajectory 230, 240 can be subject to multiple repeated passes of the cutting wire.

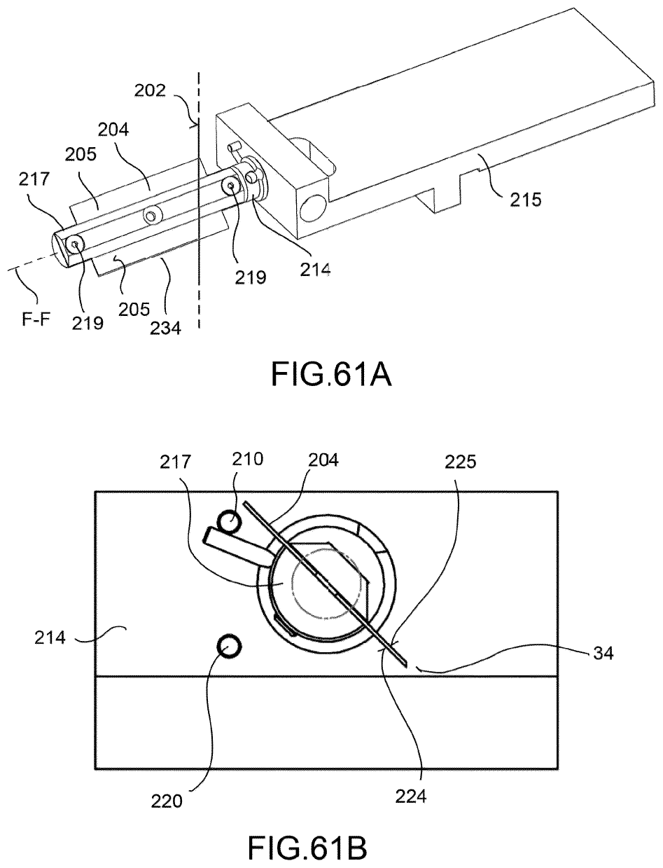

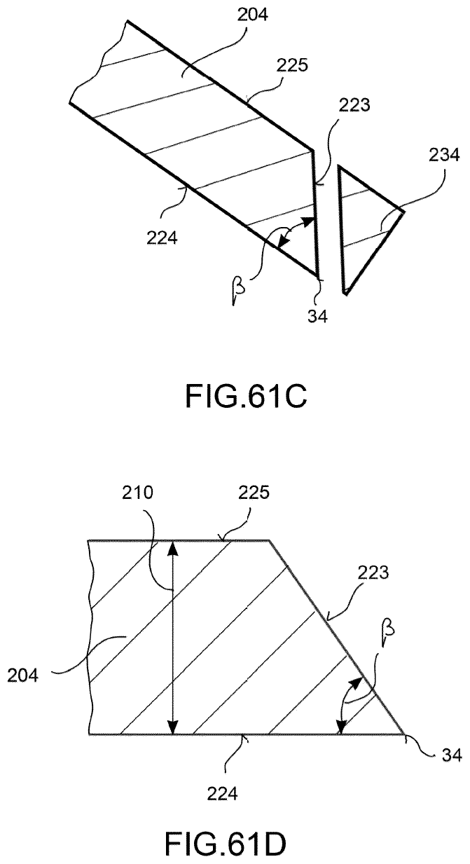

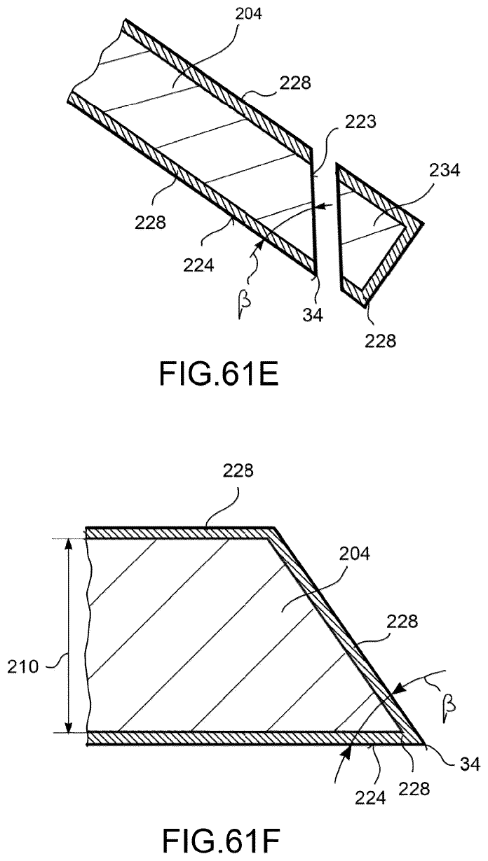

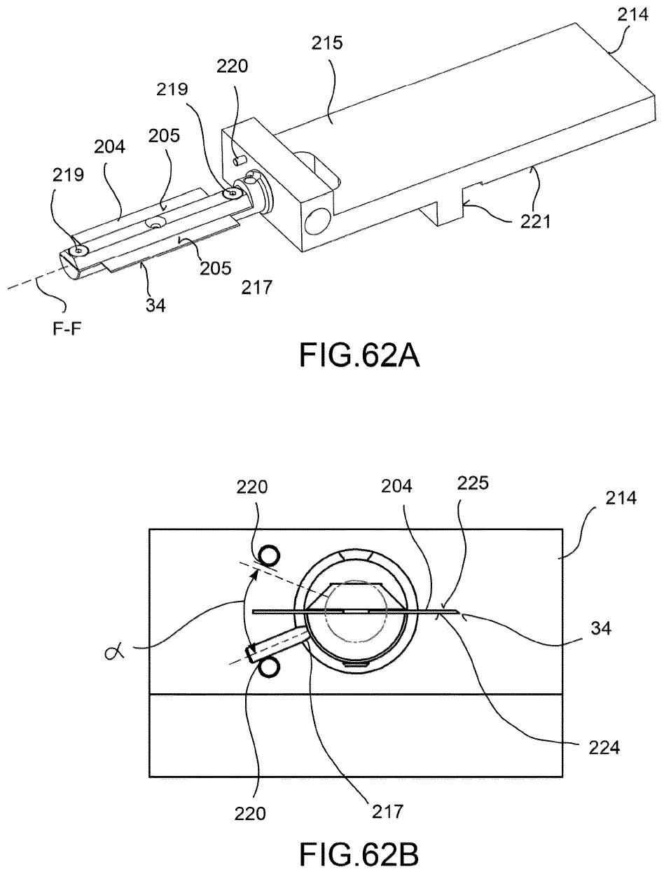

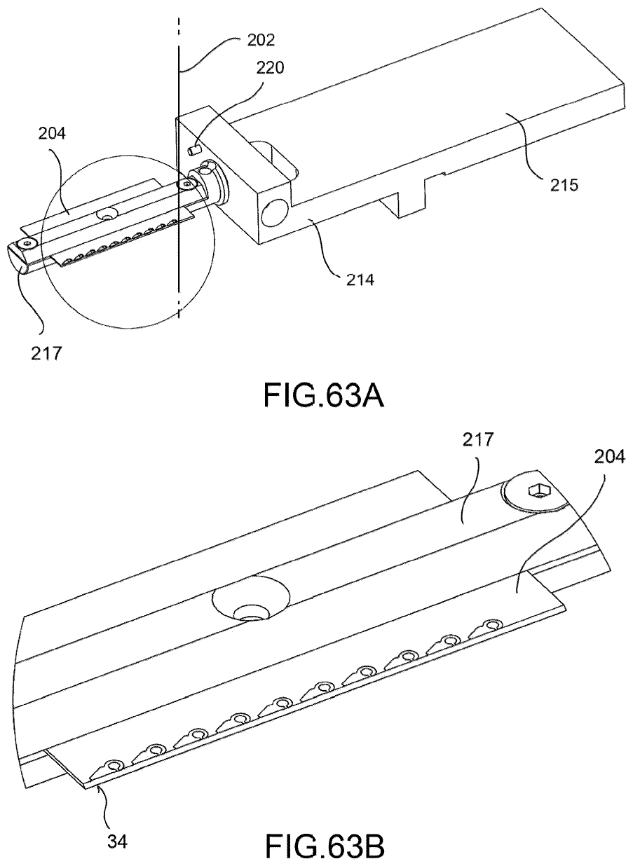

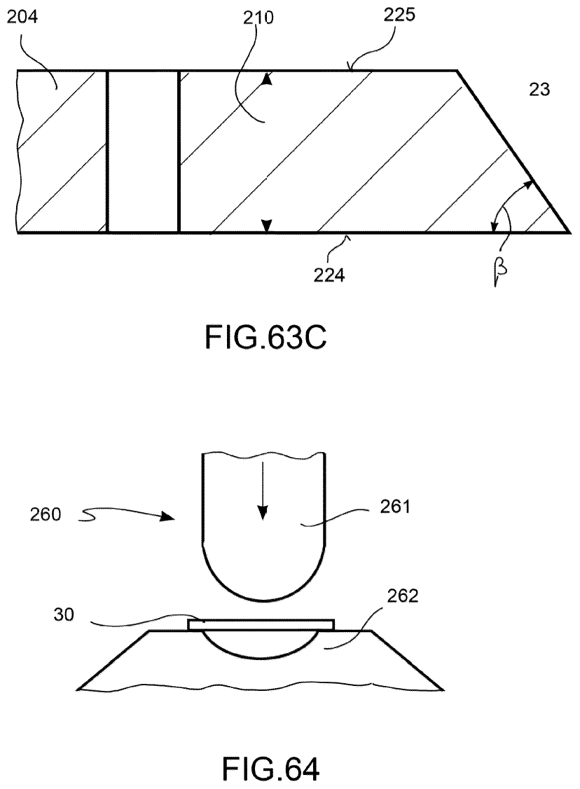

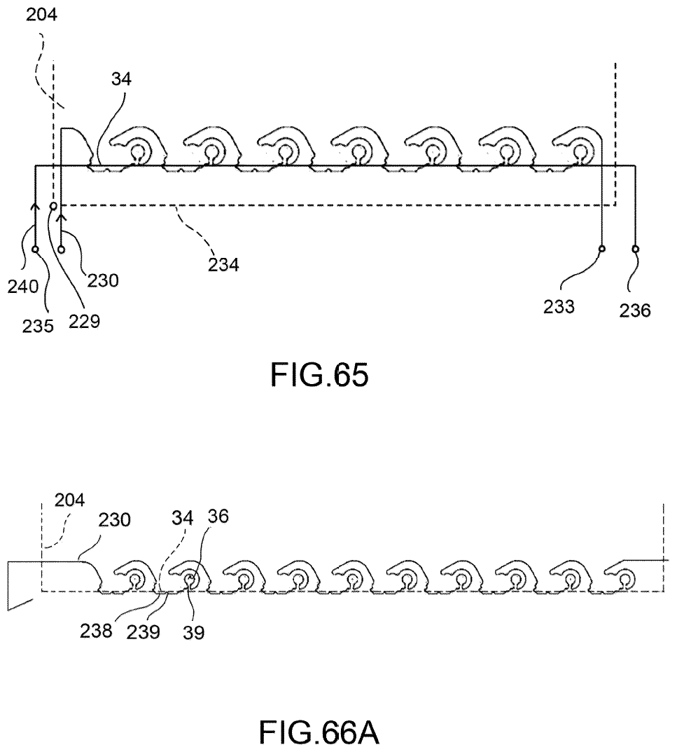

The sharpening through cut removes material from an edge to be sharpened 234 of the workpiece, exposing a sharpening cutting wall 223, in a condition in which the workpiece 204 and the cutting wire 202 form a certain angle therebetween (which depends on the choice of the sharpening angle α) chosen so that the exposed sharpening cutting wall 223 and another wall of the workpiece adjacent thereto jointly form a cutting edge 34, i.e., an acute-angled edge defined by the meeting of the sharpening cutting wall 223 and by said other adjacent thereto the workpiece wall. In cross-section, as shown in FIG. 61-C, for example, following the sharpening through cut, the sharpening cutting wall 223 forms an acute angle (preferably with a face 224 of the back side of the workpiece 204. The sharpening cutting wall 223 can form an acute angle with an opposite face 225, i.e., the front side of the workpiece 204.

Such an acute angle β formed between the sharpening cutting wall 223 and another wall of the workpiece 204 does not necessarily correspond to said sharpening rotation angle α, although in accordance with an operating mode said sharpening rotation angle α is equal to said acute angle β. In accordance with an embodiment, the acute angle ß is equal to 90°-α.

In accordance with a possible operating mode in which the workpiece has a plate-like body with parallel opposite faces 224, 225 defining a thickness 210 therebetween, the shaping through cut is performed perpendicularly to the opposite parallel faces 224, 225 through the thickness, and the sharpening through cut is performed in an inclined direction with respect to the opposite parallel faces 224, 225 and across the thickness of the workpiece. Thereby the cutting edge 34 is formed on one face of the opposite parallel faces 224, 225 of the workpiece 204 which is transverse (in this case orthogonal) to the shaping cutting plane and incident to the sharpening cutting plane.

Where the workpiece 204 has a certain geometry, for example but not limited to a planar strip or ribbon or sheet geometry given by the plate-like body thereof, and said sharpening rotation angle α is understood as the rotation angle of the plate-like body during the rotating step, then the acute angle β is in accordance with a preferred embodiment equal to or complementary to the sharpening rotation angle α.

The workpiece 204 can have a squat body or other non-plate-like body and the sharpening through cut is performed through the body of the workpiece 204, forming said cutting edge 34.

The acute angle of the cutting edge 34 must be chosen so as to optimize the cutting performance, finding a compromise between penetration and strength. Typically, an acute angle ß of the cutting edge 34 of less than 45°, for example between 10° and 40°, allows for high cutting penetration but tends to wear out early (trend which increases with decreasing acute angle β amplitude) while an acute angle ß of the cutting edge 34 greater than 45°, for example between 50° and 80°, allows for long service life but the cutting edge 34 can register resistance to cutting penetration when in service conditions (trend which increases with decreasing acute angle ß amplitude). An acute angle ß in the range from 30° to 60° (values to be understood here with a tolerance of +10%) would offer a satisfactory compromise for applications of the resulting one or more blade(s) 30 in the field of robotic surgery.

In accordance with a preferred embodiment, the acute angle β is substantially equal to 45°. This value can also be understood here with a tolerance of +10%, although it is preferable here to indicate an acute angle (which is substantially equal to half of 90°, i.e., it makes a through cut exposing a cutting wall significantly facing 45° in the workpiece body. Accordingly, where the acute angle β depends on the sharpening rotation angle α, said sharpening rotation angle α can be in the range of 20°-70°, and preferably the sharpening rotation angle α is substantially 30°±10° or 45°±10° or 60°±10°. These values are to be understood in absolute value, i.e., they can be valid in any rotation direction of the body of the workpiece 204 with respect to the cutting wire 202 made during the rotation step. Therefore, 45° here means a rotation of 45° in one direction and also an equal rotation of 45° in the opposite rotation direction. The rotation direction has an effect on the direction of the cutting wall 223 exposed on the body of the workpiece 204 and can determine whether the cutting edge 34 belongs to the face of the back side 224 or to the face of the front side 225 of the workpiece 204.

The sharpening angle α can be chosen to minimize the distance between the workpiece and a reference of the machine 200, for example a head 208.

In accordance with a possible operating mode, the sharpening through cut of the sharpening step follows a cutting path 240 extending along the thickness to be sharpened 234 of the workpiece 204. Thereby, it allows making a substantially uniform cutting edge 34 along the extension thereof even where the edge to be sharpened 234 has a concave and/or convex geometry in the sharpening cutting plane.

In accordance with a possible operating mode, the edge to be sharpened 234 of the workpiece 204 coincides with an edge of the workpiece body, for example an edge of the plate-like body such as a strip or plate or ribbon and the cutting path 240 of the sharpening through cut extends substantially straight, along the edge of such a margin, and substantially files the edge i.e., electro-erodes material from the thickness 210 of the plate-like body of the workpiece, making a gap which exposes a cutting surface 223 which is inclined with respect to the opposite faces 224, 225 of the plate-like body and forms a cutting edge 34.

Choosing the sharpening rotation angle α can define the direction of the sharpening and shaping through cuts on the workpiece.

In accordance with a preferred operating mode, the shaping through cut crosses the body of the workpiece 204 in the direction of the thickness thereof. In accordance with a preferred operating mode, the shaping through cut produces an edge which is not sharp and for example forms two opposite angles of substantially 90° with the opposite faces 224, 225 of the workpiece, where the workpiece has a predefined regular geometry, for example it is a plate-like body. The cutting path 230 described by the shaping through cut can form a path comprising curved portions, such as hole edges 36, and in accordance with a possible operating mode making the hole edges 36 involves making radial passage channels 39 for the passage of the cutting wire. The hole edges 36 are not necessarily formed by curved portions and can be formed by broken line segments of hole edges 36. The hole edges 36 can delimit one or more centering holes for receiving an articulation pin when in operating condition. The curved portions described by the cutting path 230 described by the shaping through cut can make the edge to be sharpened 34 so as to make a curved, concave, and/or convex edge to be sharpened. The feeding speed parameters of the cutting wire 202 can be adjusted to provide a good compromise between finishing and production times. In accordance with an embodiment, the shaping step makes parts with extreme resolution by means of said through cut, such as legs measuring a few hundredths of a millimeter in width.

In accordance with a possible operating mode, the shaping through cut makes an edge which is not orthogonal with respect to the opposite faces 224, 225 of the workpiece 204, i.e., the shaping cut can make an inclined edge with respect to a definable lying plane of the workpiece.

In accordance with a possible operating mode, first the sharpening step is performed, then the rotating step, then the shaping step. Thereby, the sharpening is done, and the shaping after. In this case, the shaping through cut can cross at least one portion of the sharpening through cut, i.e., the shaping cutting path 230 is incident with the sharpening cutting path. In accordance with this operating mode, the method can allow making a plurality of blades, for example a plurality of blade links 30, from the same workpiece by first sharpening at least one portion of at least one edge of the workpiece 204 which is common to, i.e., is shared by, at least one group of blades to be made, and then shaping the individual blades which includes performing a shaping through cut which crosses the cutting edge 34 and thus cuts the cutting wall 223 to make the individual blades obtainable from the same workpiece 204 separate or separable. For example, where the workpiece is a plate-like body mounted on the jig 214 forming two opposite cantilevered edges, the method can include first sharpening both said edges and then shaping the individual blades of said plurality on both opposite cantilevered flaps.

In accordance with a possible operating mode, the sharpening step is performed before the shaping step, and in which the shaping cutting path 230 of the shaping step does not extend along the cutting edge 34 made by the sharpening step, i.e., the shaping through cut is not made on the workpiece following the profile of the cutting edge 34 previously machined. The cutting path 230 of the shaping through cut can cross the cutting edge 34 transversely with respect to the longitudinal extension of the edge to shape the blades 30, making an interruption of the cutting edge of the workpiece 204.

In accordance with a possible operating mode, the cutting path 230 of the shaping through cut includes an external section 238 of the cutting path 230 of the workpiece 204 in an external position with respect to the cutting edge 34 and at a certain distance therefrom, in which a calibration verification step is carried out along the external portion 238 of the cutting path 230 which includes a sudden approach of the cutting wire to the cutting edge 34, substantially tracing a notch 239 on the cutting path 230. Thereby, it allows verifying the correct positioning of the workpiece 204, in fact where the sudden approach of the cutting wire 202 to the cutting edge 34 results in the electro-erosion of material from the cutting wire 202 this would indicate an anomaly, for example a probable positioning error of the workpiece.

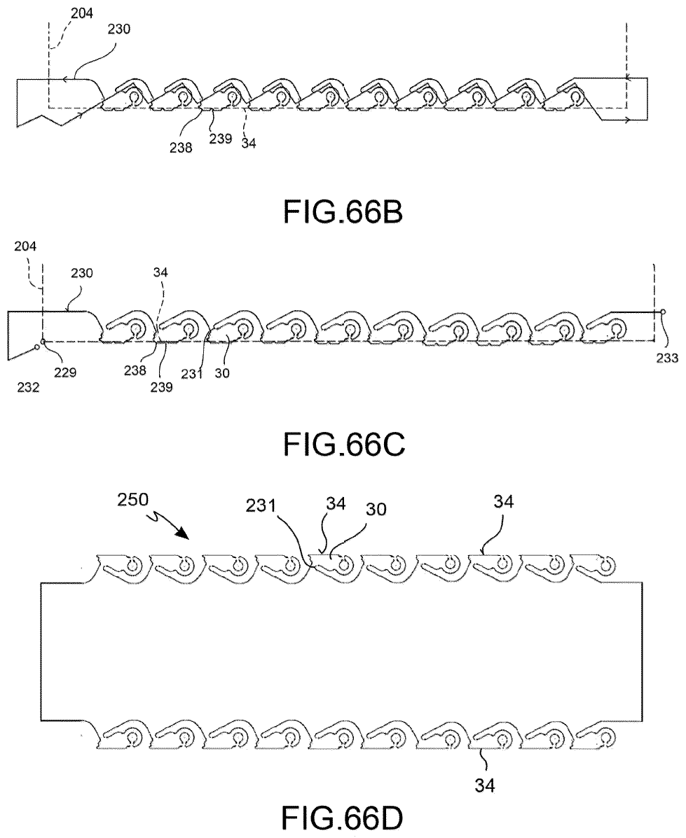

FIG. 66-B shows an example of a shaping cutting path 230 of a shaping through cut which describes the shape of a plurality of blades 30 on the same workpiece, making undercuts, hole edges 36, passage channels 39, said external section 238 with respect to the cutting edge 34. The shaping cutting path 230 shown here can be performed several times, i.e., with multiple repeated passes, for example round trip passes.

FIG. 66-B shows an example of a shaping cutting path 230 of a shaping through cut which includes different round trip paths which intersect, resulting in the shaping and separation of a plurality of blades 30. In accordance with a possible operating mode, the cutting profile 230 shown in FIG. 66-B can be understood as a single return path to the at least one outward path shown in FIG. 66-A, and in such a case the single return path machines substantially straight edges of the blade bodies and the shaping through cut performed along said single return path of the shaping cutting path 230 performs the function of separating the blades. In accordance with a possible operating mode, the cutting profile 230 shown in FIG. 66-B can be understood as a shaping cutting profile independent from the one shown in FIG. 66-A and the round trip path can be chosen if necessary.

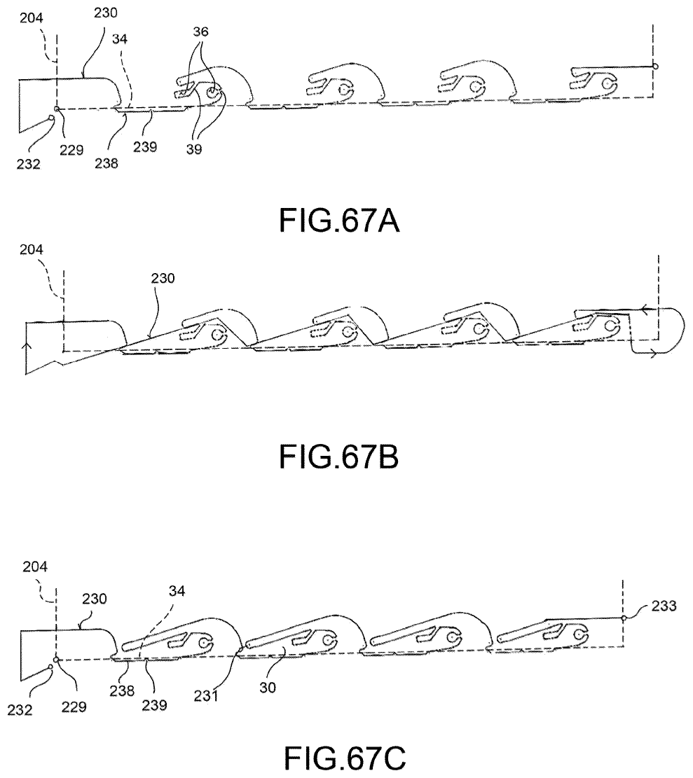

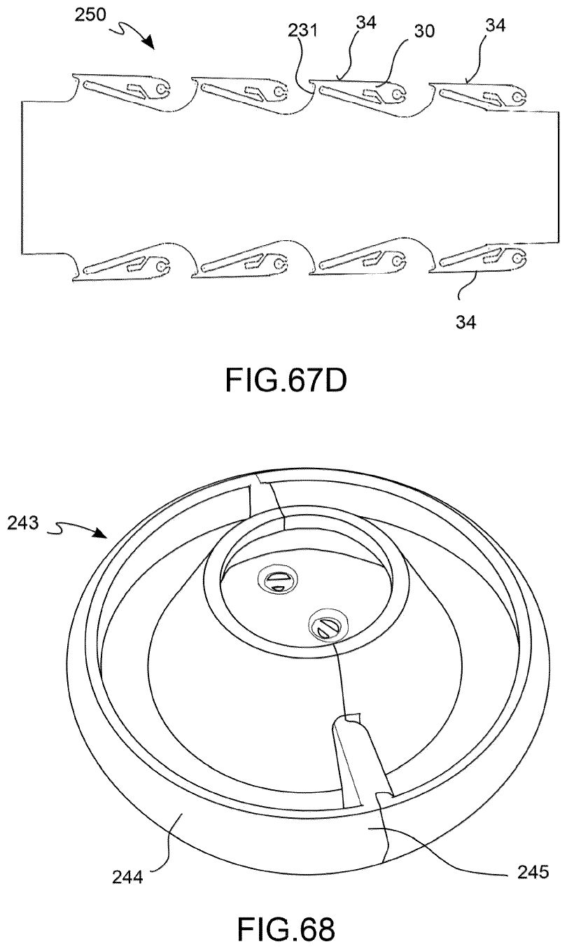

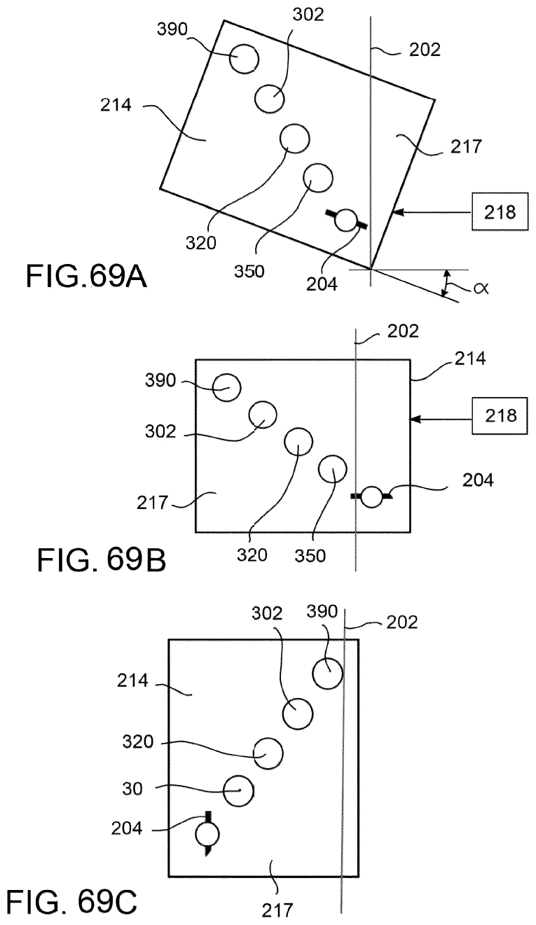

FIGS. 67-A and 67-B show an example similar to that shown in FIGS. 66-A and 66-B described above.

The sharpening cutting path can be performed multiple times i.e., with multiple repeated passes, e.g., round trip passes, e.g., in a number between 3 and 11 passes, and preferably between 3 and 7 passes. In accordance with an operating mode, said sharpening cutting path of the sharpening step is performed more often than the shaping cutting path of the shaping. This results in a better finish of the cutting edge 34. In accordance with a preferred operating mode, the sharpening cut is performed before the shaping cut so that during the process of making the blade the piece is not subjected to vibrations during the first or the multiple finishing passes.

The shaping cut is preferably also detaching, i.e., it results in the separation of the blade 30, and is preferably performed after making the blade and preferably in a single pass.

In accordance with a possible operating mode, the sharpening step is performed by a single cutting sharpening trajectory 240 of the cutting wire 202 and said shaping step is performed by a single cutting shaping trajectory 230 of the cutting wire 202. Preferably, the sharpening cutting path or trajectory 240 has a starting point 235 and an ending point 236, which can be coincident if an even number of round trip passes are performed. Preferably, the shaping cutting path or trajectory 230 has a starting point 232 and an ending point 233, which can be coincident in the case in which an even number of round trip passes are performed.

A basket 243 for collecting the blades 30 which are separated can be provided, as shown in FIG. 68, for example. For example, the basket 243 is made of two separable half-bodies 244, 245 which can be assembled, for example interlocked, around the lower head 206 of the electro-erosion machine 200, forming when assembled at least one collection chamber having a substantially annular shape to collect the separate blades 30 which, due to the effect of gravity, fall into the dielectric liquid tank 208. In such a case, the method can comprise, after the step of separating the blades 30, the step of collecting by gravity the sharpened, shaped and separated blades 30 by wire electro-erosion.

FIG. 66-C and FIG. 67-C each show an example of a shaping cutting path 230 of a shaping through cut which describes the shape of a plurality of blades 30 on a same workpiece, each provided with a connection bridge 231, making undercuts, hole edges 36, passage channels 39, said external section 238 with respect to the cutting edge 34. The shaping cutting path 230 shown here can be performed several times, i.e., with multiple repeated passes, for example round trip passes. In such a case, the method can include the step of separating the blades 30 comprising breaking the breakable connection bridges 231 to be performed elsewhere and for example the step of separating the blades by breaking the connection bridges 231 can be carried out during the assembly of the finished product, such as a surgical cutting instrument.

FIGS. 66-D and 67-D show some examples of a semi-finished product 250 made with a method according to any one of the operating modes described herein comprising a plurality of blades each provided with a connection bridge 231, for example made of breakable material. In accordance with an operating mode, the method further comprises the step of making said semi-finished product 250 and the step of separating the blades by breaking the respective connection bridges 231.

The step of breaking the connection bridges 231 can be performed by wire electro-erosion, making a shaping cut.

In accordance with a possible operating mode, first the shaping step is performed, then the rotation step, then the sharpening step. Thereby, the shaping is done first, and the shaping after.

This possible operating mode is preferably performed if the connection bridge, the shape of the piece or the thickness of the piece itself are sufficient not to induce vibrations during the one or more sharpening passes on the already shaped piece.

In accordance with a possible operating mode, the shaping step is performed first, then the rotating step, then the sharpening step, then a further rotating step and then a further shaping step, i.e., the shaping step can be partially performed before the sharpening step and completed after the sharpening step. In accordance with this operating mode, the shaping step can leave the shapes of the one or more blades traced by cutting on the workpiece but interconnected by bridges of material 231, for example breakable bridges of material of locally reduced thickness.

In accordance with an embodiment, the method determines the manufacturing of a semi-finished product 250 comprising a plate-like body in which a plurality of blades is shaped, for example a plurality of blade links 30, each having a cutting edge 34 in which the blade bodies are mutually interconnected by one or more material bridges 231 of the workpiece body which has not been intentionally removed, for example breakable material bridges.

In accordance with a possible operating mode in which the shaping step is performed first, then the rotating step, then the sharpening step, and in which the shaping makes shapes on the workpiece 204 of the one or more cuttingly shaped blades (but without a cutting edge 34) and interconnected by material bridges 231, the sharpening step can be performed on the edges to be sharpened 234 of the individual blade shapes, although the cutting path can still follow a continuous path which in some sections does not cross material of the workpiece which has already been removed, for example, from the shaping through cut.

In accordance with a possible operating mode, the sharpening and shaping steps can alternate and a rotation step is always included therebetween.

Multiple sharpening cuts on different cutting planes and/or multiple shaping cuts on different cutting planes can be included. For example, a step of rotating the jig between two adjacent sharpening steps can be included, and/or a step of rotating the jig two adjacent shaping steps can be included. For example, between two shaping cuts of the same workpiece, a rotation angle of the jig 214 of substantially 90° C. an be included, even if between said two shaping cuts a sharpening cut at another, further orientation is included.

For example, between two sharpening cuts of the same edge to be sharpened of the same workpiece, a rotation angle of the jig 214 greater than or equal to 90° C. an be included, albeit in order to make an acute angle β in the body of the workpiece 204. In accordance with a possible operating mode, two sharpening through cuts are made on two cutting planes rotated therebetween by 90°-150° and preferably 120°-150°.

In accordance with a possible operating mode, the method comprises the step of separating said one or more blades. The separating step can be included in the shaping step, where the cutting path of the shaping through cut makes one or more separate blades. Where a semi-finished product 250 is produced in which a plurality of blades each having a cutting edge 34 in which the blade bodies are interconnected by one or more material bridges 231 is cuttingly shaped, the separating step can comprise breaking said material bridges 231 and could also be performed at the assembly site.

In accordance with a possible operating mode, the workpiece 204 is an elastic body having an elastically deformable body for exerting an elastic reaction. In accordance with an embodiment, the workpiece 204 is an elastic plate-like body, for example it is an elastic strip adapted to bend elastically. The provision of an elastically bendable workpiece allows making a miniaturized elastic blade having an elastically bendable body.

Preferably, the workpiece 204 is made of metallic material. The workpiece 204 can be made of steel for blades. One or more surface treatments 228 on the workpiece can be included, such as coatings and/or heat treatments, for example to make the cutting edge 34 harder and more resistant to wear when in operating conditions. In accordance with an embodiment, the cutting edge 34 comprises a surface treatment 228 at least on the surface 35 intended to work by mechanical interference contact against a counter-blade when in operating conditions.

The workpiece 204 can be subjected to bending such as by press-bending, for example as shown in FIG. 64. In such a case, the method comprises the step of bending the blade, for example a blade portion 14 and/or a blade link 30. This step can comprise the step of including a press 260, for example having a hammer 261 and an anvil 262. Bending by press-bending can be performed to give the blade 30 elastic properties.

In accordance with a possible operating mode, the method comprises the step of treating the surface of the workpiece, obtaining a surface treatment 228 on the workpiece. The step of treating the surface can also be performed more than once.

In accordance with a possible operating mode, the step of treating the surface is performed before the sharpening step. Where the surface treatment 228 is carried out before said sharpening step, then the wall 223 exposed by the flush cut of the cutting edge 34 will lack surface treatment 228. In this case, for example, a “no-back-bevel” or “chisel edge” type sharpening can be obtained in which the surface 35 of the cutting edge 34 intended to work by mechanical interference contact against a counter-blade when in operating conditions comprises a surface treatment 228 while the opposite cutting wall 223 does not comprise any surface treatment 228.

In accordance with a possible operating mode, the step of treating the surface is performed after the sharpening step. Where the surface treatment 228 is carried out after said sharpening step, then the wall 223 exposed by the flush cut of the cutting edge 34 can comprise a surface treatment 228.

In accordance with a possible operating mode, the step of treating the surface comprises the step of making a diamond-like-carbon (DLC) type coating.

In accordance with a possible operating mode, the step of treating the surface comprises the step of carrying out a heat treatment, for example of the “kolsterizing®” type.

In accordance with an operating mode, the step of coating the surface is performed when the workpiece is in the form of a semi-finished piece 250 having a body comprising in a single piece a plurality of shaped blades interconnected by connection bridges 231. Thereby, the miniaturization of the blades is facilitated because it allows positioning a plurality of blades together for surface treatment, by positioning the body of the semi-finished piece 250, for example a ribbon or strip.

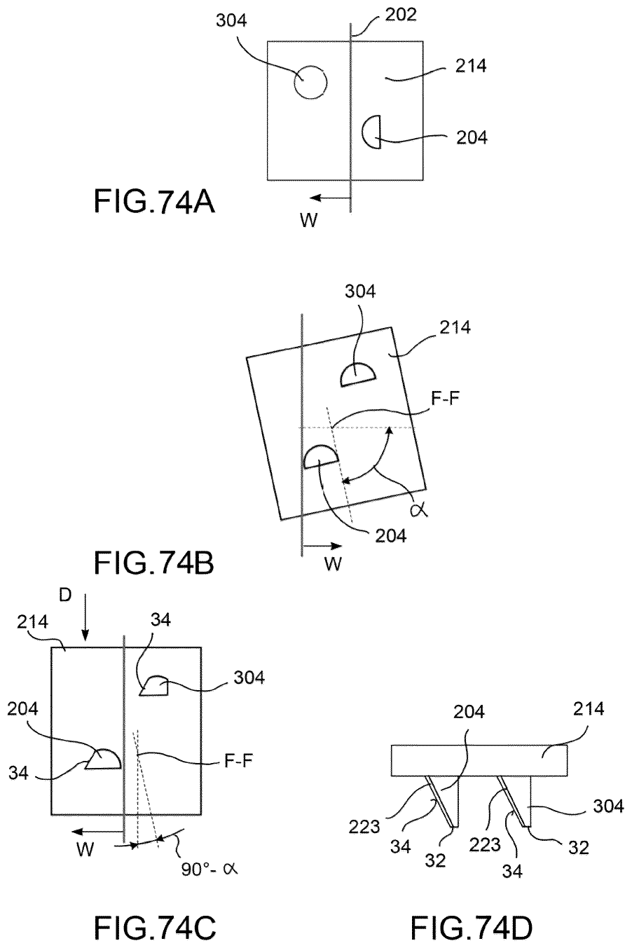

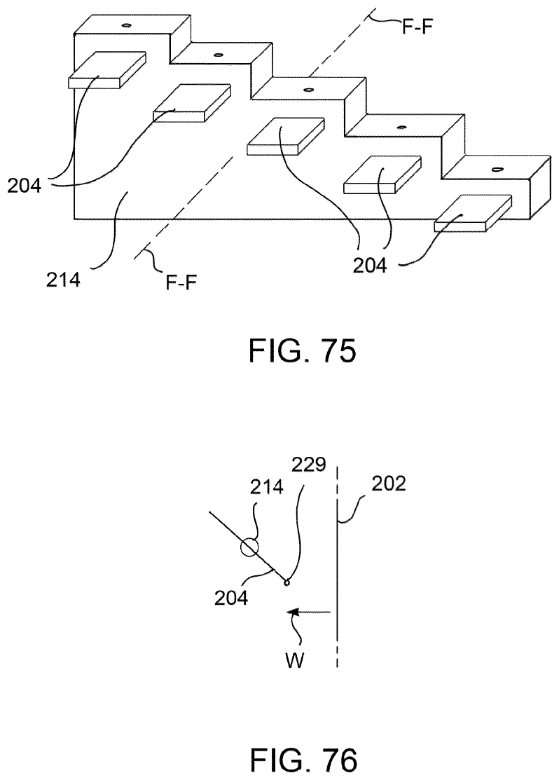

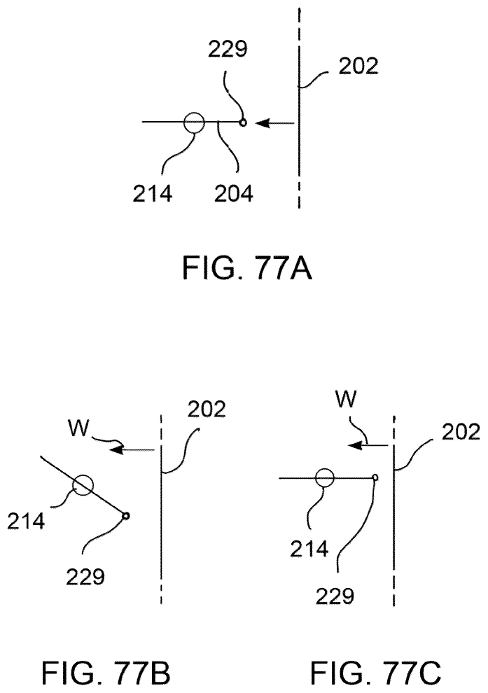

In accordance with a possible operating mode, the method further comprises after the shaping step, the further reshaping step to make a second shaping, on a second cutting plane, said workpiece 204, performing with the cutting wire 202 a second shaping through cut on the at least one workpiece 204, in which between the shaping step and the reshaping step, the step of rotating said fixture 214 by a shaping angle preferably substantially equal to 90° is included. In accordance with this operating mode, preferably the shaping step is performed before the sharpening step. As shown, for example, in the sequence of FIGS. 74A-C, it is possible to carry out first the shaping step, then the sharpening step, then the reshaping step, in which between the shaping step and the reshaping step the workpiece 204 has been rotated by rotation of the fixture or a portion thereof at an angle substantially equal to 90°.

Between the shaping step and the sharpening step, the workpiece 204 can be rotated by a sharpening angle α.

Thereby it is possible to perform two shaping cuts and one sharpening cut on the same workpiece 204.

In accordance with a possible operating mode, the mounting step comprises mounting on said fixture 214 a plurality of workpieces 204, 304, and in which the sharpening and shaping steps comprise individually sharpening and shaping each workpiece. In other words, in accordance with this operating mode, each workpiece 204, 304 is machined individually, avoiding performing simultaneous cuts on a multiplicity of workpieces. Where different cuts are made on different pieces, said cuts can be made in succession on the different pieces.

In accordance with a possible operating mode, the mounting step comprises mounting on said fixture 214 also at least a second workpiece 304 so as to obtain at least two workpieces 204, 304 mounted on the same fixture 214, and in which the method further comprises sharpening at least one edge to be sharpened of said second workpiece 304, and in which between the sharpening step at least one edge to be sharpened of the at least one workpiece 204 and the step of at least one edge to be sharpened of said second workpiece 304 a further step of rotating at least one portion of said fixture 214 is included. Thereby it is possible to obtain different sharpnesses on different workpieces 204, 304.

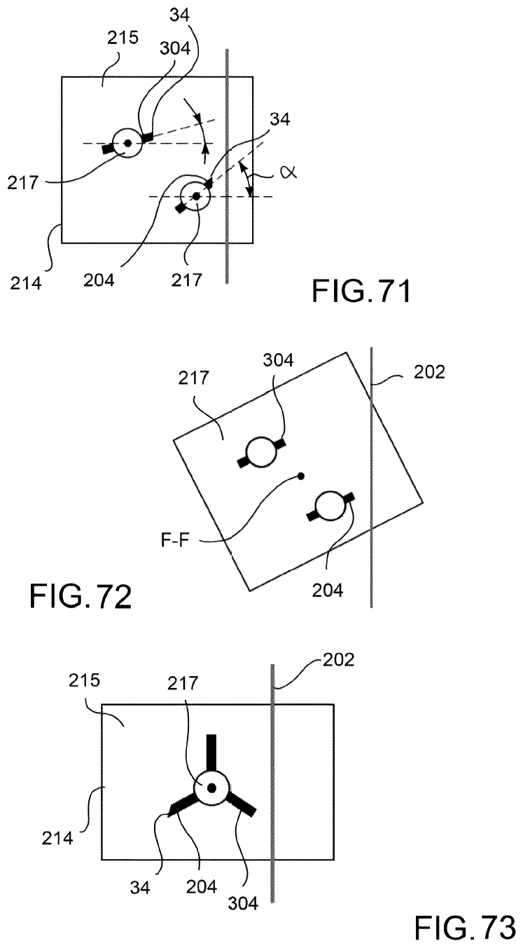

As shown in FIG. 71, for example, two sharpening cuts can be made on different workpieces by rotating the housing portion 217 which mounts each workpiece 204, 304 by a different sharpening angle, i.e., a rotation of a first workpiece 204 by a first sharpening angle α and a rotation of a second workpiece 304 by a second sharpening angle α2. Thereby, it is possible to make sharp edges having different acute angle β on different workpieces 204, 304.

As shown in FIG. 72, for example, it is possible to make two sharpening cuts on different workpieces 204, 304 integral in rotation with each other by including between two sharpening steps, i.e., between the step of sharpening at least one edge to be sharpened of the at least one workpiece 204 and the step of sharpening at least one edge to be sharpened of said second workpiece 304, a further step of rotating at least one portion of said fixture 214 by a certain angle, for example equal to α2-α. The angles α and α2 can be different from each other by any amount. The angle α2 can be chosen according to the same considerations set forth with reference to the angle α and thus with reference to the direction of the cutting wire 202 for performing a shaping cut.

In accordance with a possible operating mode, the fixture 214 receives a plurality of workpieces 204 having a plate-like body arranged so as to be individually and singularly machinable by the cutting wire 202, in one or more rotation configurations of the fixture 214.

As shown in FIG. 73, for example, three (or more) workpieces 204 having a plate-like body can be star-shaped on the fixture 214, i.e., can be arranged to extend with a respective cantilevered flap from the housing portion 217 of the fixture 214 in radial directions with respect to the housing portion 217. For example, the workpieces in star configuration can be individually sharpened and between the sharpening of one workpiece and another, a step of rotating the housing portion 217 of the fixture 214 can be included.

In accordance with an embodiment, the fixture 214 or jig 214 includes fixing multiple planar elements (strips), which can be machined individually by electro-erosion in one or more rotation configurations.

In accordance with a possible operating mode, the method comprises at least two shaping steps, i.e., a shaping step and a reshaping step, and between said two shaping steps the further step of rotating the jig 214 by a shaping angle which is preferably substantially equal to 90° is included. In other words, preferably, the two shaping steps are performed on two cutting planes orthogonal to each other. It is also possible for the method to first include a first shaping step, then rotate the jig 214 by said sharpening rotation angle α (e.g.,)α=40° and perform a sharpening step, then rotate the jig 214 again by an angle equal to 90°—a (in this example 50°) and perform a second shaping step, in which from the first shaping step to the second shaping step the jig 214 has rotated by 90°.

This operating mode can be advantageous for producing with a single placement of the workpieces in the electro-erosion machine 200 an assembly of links to be mutually assembled of an articulated end-effector of a surgical cutting instrument (e.g., a surgical scissor or needle-driver/scissors), in which at least one of the links of the link assembly has a cutting edge 34 and for example is a blade link 30 and/or is a tip link 10 comprising a blade portion 14.

In light of the above, a method of manufacturing a plurality of links of an articulated end-effector 9 for a surgical cutting instrument 1 by wire electro-erosion will be described below.

Said articulated end-effector 9 is preferably actuatable by means of actuation tendons. Said articulated end-effector 9 can be an articulated end-effector according to any one of the embodiments described above.

In accordance with a general embodiment, a method of manufacturing a plurality of links of an articulated end-effector 9 by wire electro-erosion comprises the steps below.

This method comprises the step of providing a wire electro-erosion machine 200 comprising a cutting wire 202 and a jig 214 which is rotatable with respect to the cutting wire about a rotation axis F-F which is transverse to the longitudinal extension of the cutting wire.

This method comprises the step of mounting a plurality of workpieces 204, 302, 320, 350, 390 all integral in rotation with the jig 214 so that the cutting wire 202 intersects at most one of said workpieces 204, at a time. In other words, the workpieces are mounted on the jig in such an arrangement (for example, they are mutually aligned at a certain distance between two adjacent pieces, or they are arranged on a curved line) that they can be machined by the cutting wire 202 singularly, i.e., individually, avoiding cutting more than one workpiece at the same time. Said plurality of workpieces can comprise pieces to be shaped 302, 320, 350, 390 intended to be shaped on two cutting planes and not sharpened, and workpieces 204, 304 intended to be sharpened and also shaped. The pieces to be shaped 302, 320, 350, 390 can be cylinders which are mounted on the jig 214 so that they protrude cantilevered, for example in a direction parallel to the rotation axis F-F.

This method can make all the links of the articulated end-effector 9 (e.g., an articulated cuff) of the surgical instrument 1. The pieces to be shaped 302, 320, 350, 390 are in accordance with an embodiment intended to form the links 2, 20, 50, 90 of the articulated end-effector 9 described above, and in particular said connection link 90, said support link 2 comprising said support structure, said second tip link 20, said blade holder link 50 of the first tip 10.

This method can make a sub-group of links of the articulated end-effector 9. The pieces to be shaped 320, 350 are in accordance with an embodiment and intended to form the links 20 and 50 of the articulated end-effector 9, and in particular said second tip link 20 and said blade holder link 50 of the first tip 10.

This method further comprises the step of sharpening at least one edge 234 of at least one workpiece 204 of said plurality of workpieces by performing with the cutting wire 202 of a sharpening through cut on the at least one workpiece 204, and the step of shaping on a first cutting plane at least some of, and preferably all, the workpieces of said plurality of workpieces by performing a shaping through cut with the cutting wire 202 on at least some of, and preferably all, the workpieces, one at a time in succession.

Between the sharpening step and the shaping step on a first cutting plane, the further step of rotating the jig 214 about the rotation axis F-F thereof by a sharpening rotation angle α other than 90° in absolute value is performed. As for the sharpening angle α, one or more of the considerations described above can apply.

This method further comprises the further step of reshaping on a second cutting plane at least some, but also all, of the workpieces of said plurality of workpieces by performing a shaping through cut with the cutting wire 202 on said at least some workpieces of said plurality, one at a time in succession.

Between shaping step on a first cutting plane and the shaping step on a second cutting plane, the step of rotating the jig 214 about the rotation axis F-F thereof by a rotation angle substantially equal to 90° is included. As explained above, depending on the order which can be arbitrarily chosen of the steps of sharpening, shaping on a first cutting plane and shaping on a second cutting plane, this rotating step by a rotation angle substantially equal to 90° C. an be operatively performed in two moments, in which one of the two execution moments corresponds to the step of rotating the jig 214 about the rotation axis F-F by a sharpening rotation angle α.

The arrangement of the workpieces of said plurality of pieces to be machined on the jig preferably must meet the condition that the cutting wire 202 intersects at most one of the workpieces at a time in each step (sharpening, first shaping, second shaping). For example, where only one of the workpieces is to be subjected to the sharpening step, such a workpiece 204 can be arranged at the edge of a row according to which the workpieces of the plurality of workpieces are arranged.

The housing portion 217 of the jig 214, i.e., the part of the jig which is rotatable with respect to the fixing portion 215, in this embodiment preferably comprises a plurality of housing seats 241 integral in rotation with one another. Preferably, the housing seats 241 are mutually aligned.

In accordance with a possible operating mode, the shaped pieces as well as the sharpened pieces are assembled together. Therefore, the method can comprise the step of assembling the pieces obtained together.

In accordance with a possible operating mode, the shaping step and/or the reshaping step comprises shaping two workpieces differently. In accordance with a possible operating mode, the shaping step comprises shaping two workpieces so that one portion of a shaped piece is complementary to one portion of another shaped piece.

In accordance with a possible operating mode, the rotating step comprises providing a rotating support table and rotating said rotating support table. The rotating support table is preferably integral with at least one and preferably all the workpieces.

In accordance with a possible operating mode, the method is performed by providing at least some workpieces of said plurality in the form of material cylinders, for example said pieces to be shaped 302, 320, 350, 390 are material cylinders which are mounted on the jig 214 so that they protrude cantilevered and in which the shaping and reshaping steps create 90° edges on said cylinders. In other words, the shaping and reshaping steps remove material from the curved side face of the cylinders, creating orthogonal faces.

In accordance with a possible operating mode, the method makes three links of the articulated end-effector to be assembled together, in which at least one link is a link comprising a cutting edge 34, and the housing portion 217 of the jig 214 comprises three housing seats 241 integral in rotation with one another. For example, said three links are: said blade link 30 having said cutting edge 34, said blade holder link 50 and said second tip link 20 comprising said counter-blade surface 24.

It is also possible that two links are obtained from a single workpiece and in such a case the method can make a plurality of links of the articulated end-effector 9 to be assembled together, in which at least one link is a link comprising a cutting edge 34, and the housing portion 217 of the jig 214 comprises at least two housing seats 241 integral in rotation with each other. For example, the blade holder link 50 and the second tip link 20 can be manufactured from the same workpiece.

In accordance with a possible operating mode, the method makes five links of the articulated end-effector to be assembled together, in which at least one link is a link comprising a cutting edge 34, and the housing portion 217 of the jig 214 comprises five housing seats integral in rotation with one another. Where two links are obtained from a single workpiece and in such a case the method makes five links of the articulated end-effector 9 to be assembled together, in which at least one link is a link comprising a cutting edge 34, and the housing portion 217 of the jig 214 comprises at least two housing seats 241 integral in rotation with each other.

In accordance with an operating mode, the at least one workpiece 204 for making the link having a cutting edge 34 has a plate-like body, for example it is an elastic strip, and the workpieces for making the other links have a squat body, for example they are circular-based cylinders.

Preferably the at least one workpiece 204 is machined by sharpening and one shaping and the other workpieces 302, 320 350, 390 are not machined by sharpening, so that each workpiece is machined with two through cuts on two different cutting planes, without disassembling the pieces between one cut and another, in which the through cuts are not the same for all the pieces because at least the sharpening cut on at least one piece 204 can have a different inclination than both shaping cuts globally performed.

In accordance with a general embodiment, a semi-finished product 250 is provided comprising a sheet-like body, i.e., a plate-like body in a single piece having a plurality of shaped blades connected together by one or more breakable connection bridges 231.

The semi-finished product 250 can comprise any one of the features described with reference to any one of the embodiments described above.

The semi-finished product 250 can comprise a surface treatment 228 or can be intended to receive a surface treatment 228.

In accordance with a general embodiment, a fixture 214 or jig 214 is provided for an electro-erosion machine 200.

Said fixture 214 or jig 214 comprises a fixing portion 215 for mounting the fixture 214 to the electro-erosion machine 200 and a housing portion 217 for receiving at least one workpiece 204, in which the housing portion 217 is rotatable with respect to the fixing portion 215 about a rotation axis F-F.

Preferably, the fixture 214 further comprises a motor 218 for rotating the housing portion 217 with respect to the fixing portion 215.

The fixture 214 or jig 214 can comprise any one of the features described with reference to any one of the embodiments described above.

In accordance with an embodiment, the housing portion 217 of the fixture 214 comprises
a plurality of seats for receiving a plurality of workpieces, in which the seats for said plurality of workpieces are arranged so that two orthogonal lines intersect one workpiece at a time. In other words, the seats are arranged so that when the workpieces are mounted on the jig 214, the cutting wire 202 of the electro-erosion machine 200 cuts only one of said workpieces on two orthogonal cutting planes. Preferably, the seats for said plurality of workpieces are arranged so that three lines, two lines of which orthogonal to each other and a third one inclined by a sharpening angle α, intersect only one workpiece at a time. For example, the seats are arranged on the fixture 214 so as to be mutually aligned at a certain relative distance.

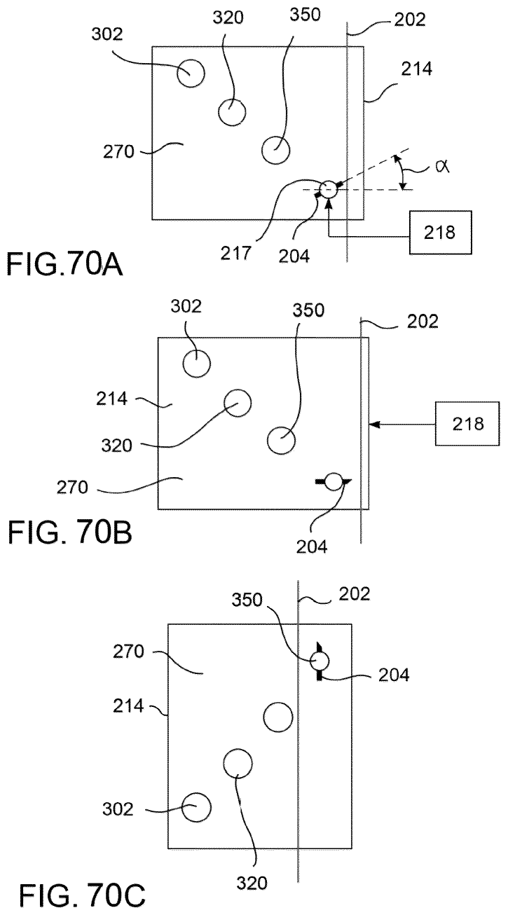

In accordance with an embodiment as diagrammatically shown in FIGS. 70A-C, the jig 214 comprises two housing portions 217, 270 which are individually or jointly rotatable with respect to the fixing portion 215 to the machine 200, in which a first housing portion 217 receives said workpiece 204 to make a sharpening cut and a shaping cut thereon, and a second housing portion 270 receives both said first housing portion 217 and one or more further workpieces 302, 320, 350 to make two orthogonal shaping cuts thereon. Preferably, the first housing portion 217 is mounted to the second housing portion 270 so that it can rotate with respect to said second housing portion about a rotation axis F-F. A single motor 218 for obtaining the rotations of the first housing portion 217 and of the second housing portion 270 can be included.

By virtue of the features described above, provided either separately or in combination in particular embodiments as well as in particular operating modes, it is possible to meet the needs mentioned above, even conflicting, and to obtain the aforementioned advantages, and in particular:
the degree of freedom of opening/closing allows performing a cutting action;
an extreme miniaturization of the articulated end-effector of the surgical instrument as compared to known solutions is allowed;
it is possible to stack the roots of the links between the prongs of the support structure, while avoiding the provision of elastic washers as well as adjustment screws, as well as tapping or threading machining at the level of the attachment roots, thus allowing an extreme miniaturization of the articulated end-effector;
in particular, the articulation pin 5 is unthreaded;
neither are the hole edges surfaces of the through holes of the roots of the respective links tapped, i.e., internally threaded, nor are the internal surfaces of the through holes of the prong through the prongs of the support structure;
there are no elastic elements, such as the “Belleville washer” type, fitted on the articulation pin;
the roots can be cylinders rigidly stacked in packs between the prongs of the support fork;
it allows providing the necessary elasticity for the cutting action in the body of the tips and outside the proximal attachment roots, i.e., away from the pin in the direction of the free ends, and particularly in the blade portion of the first tip, and if necessary, also in the counter-blade of the second tip, thus allowing a precise cutting action to be performed while creating an extremely miniaturized articulated end-effector;
in particular, for relatively high opening angles of the degree of freedom of opening/closing the blade is free, i.e., elastically non-deformed, and is preferably straight in such a configuration;
as the opening angle of the degree of freedom of opening/closing closes, the blade is elastically bent, elastically pushing against the counter-blade;
since the elasticity necessary for the cutting action is concentrated distally with respect to the roots, a deformation seat can be provided, which receives the relatively high axial bending of the blade or counter-blade:

the roots stacked in packs between the prongs provide a reaction to the elastic bending deformation of the blade, avoiding axial sliding on the articulation pin, thus allowing a precise and effective cutting action of the cutting edge;
the provision of an elastically bendable counter-blade allows making a surgical instrument of the surgical scissor type capable of a precise cutting action even at high opening angles, i.e., the cutting edge can push on the counter-blade even proximally, substantially close to the level of the roots, i.e., close to the articulation pin;
the roots stacked in packs between the prongs provide a reaction to the elastic bending deformation even where an elastically bendable counter-blade is included;
the blade link and the counter-blade link, where present, are dragged in rotation by the first tip link and the second tip link which therefore act as blade holder links and reaction links;
the provision of through holes of all the coaxial and receiving roots with contact with the articulation pin allows avoiding undesired relative rotations between the roots, providing positioning certainty of the cutting edge with respect to the counter-blade portion, thus allowing an extreme miniaturization of the articulated end-effector, since small rotational movements at the level of the root, i.e., close to the common rotation axis would impose relatively large cutting inaccuracies;
in addition, the hole of the blade link exerts with the proximal edge thereof pushing on the pin a reaction to the frictional force between the blade and the counter-blade during the cutting action, helping obtain a precise cutting action;

the cutting edge of the blade link can be made straight i.e., without concavity, facilitating production in series, for example starting from a single band or strip;

the integral rotation of the blade link and the counter-blade link, where present, with the free ends allows performing the cutting action in various orientations of the degree of freedom of yaw, so as to be able to reproduce the orientation of a surgeon's hands, thus being of a marked intuitiveness, as well as easier to view, for example, under a microscope;

the provision of an abutment of the closing stroke end distant from the articulation pin and distal with respect thereto allows a high precision in closing and at the same time does not occupy the proximal area of the support fork, favoring an extreme miniaturization;

the termination seats of the tendons and the ruled pulley surfaces made in a single piece with the respective links favor miniaturization, helping keep the number of pieces small and the articulated end-effector compact;

in case of a surgical instrument of the needle-driver/scissor type, interposing the blade between the tip links allows it to be concealed with a closed end-effector allowing, for example, to wrap the suture thread around the tip links without damaging it;

the provision of a single drag engagement portion in rotation between two links of the first tip and/or of the second tip, where present, allows minimizing the drag clearance, favoring miniaturization;

an axially rigid rotational joint is provided in which the cutting action is carried out by elements forming the rotational joint;

the rotational joint defining the common rotation axis Y-Y can be a hinge;

the reaction link can be a counter-blade holder link 60 where a separate counter-blade link 40 is included, or it can be a second tip link 20 having said counter-blade portion 24 in a single piece;

the first tip 10 can comprise a blade holder link 50 having an attachment root provided with convex ruled surfaces on which the tendons wind without sliding;

the blade holder link can comprise a gripping surface;

where at least the blade portion is made by wire electro-erosion (WEDM), it is possible to obtain excellent surface finishes of the walls made by the through cuts by wire electro-erosion and this favors a boosted miniaturization of the product of the manufacturing process; meanwhile two non-orthogonal cuts are made for shaping and sharpening the same workpiece, avoiding repositioning the workpiece and therefore further increasing the finishing:

where at least the blade portion is made by wire electro-erosion (WEDM), a “no-back-bevel” type sharpening is allowed, i.e., “chisel edge” with one or more passes of the cutting edge along a single sharpening cutting path;

where at least the blade portion is made by wire electro-erosion (WEDM), an elastic blade can be made;

where at least the blade portion is made by wire electro-erosion (WEDM), it is possible to produce a plurality of blades from a single workpiece with a single continuous cutting action, for example a plurality of blades;

where at least the blade portion is made by wire electro-erosion (WEDM), the rotation angle of the fixture from the sharpening step to the shaping step, or vice versa, is different from 90°;

where at least the blade portion is made by wire electro-erosion (WEDM), where two

shaping steps are included, the rotation angle of the fixture from the shaping step to the reshaping step is substantially 90°;

where at least the blade portion is made by wire electro-erosion (WEDM), the shaping step can comprise the step of leaving the material bridges intact, making a semi-finished product 250;

where at least the blade portion is made by wire electro-erosion (WEDM), the coating step can be performed on the semi-finished product 250 after performing the sharpening step and/or on the workpiece 204 before performing the sharpening step;

where at least the blade portion is made by wire electro-erosion (WEDM), the shaping step can comprise the step of separating the blades from the workpiece.

It is well understood that the combination of features, structures or functions disclosed in one or more of the appended claims forms an integral part of the present description.

In order to meet specific, contingent needs, those skilled in the art can make several changes and adaptations to the above-described embodiments and can replace elements with other functionally equivalent ones, without departing from the scope of the appended claims.

### LIST OF REFERENCE SIGNS

| | |
|---|---|
| 1 | Surgical instrument |
| 2 | Support link |
| 3 | First support structure prong |
| 4 | Second support structure prong |
| 5 | Pivot pin or articulation pin |
| 6 | Termination seat for the third pair of support link actuation tendons, or third support |
| | link termination seat |
| 7 | Surgical instrument shaft or rod |
| 8 | Distal shaft or rod end |
| 9 | Articulated end-effector, or articulated end device, or hinged end-effector or hinged |
| | end device |
| 10 | First tip |
| 11 | First proximal attachment root of the first tip, or first tip attachment root |
| 12 | First distal free end of the first tip, or free end of the first tip |
| 13 | First gripping surface of the first tip, or first tip gripping surface |
| 14 | First tip blade portion |
| 15 | First termination seat of the first tip |
| 16 | First through hole of the first root of the first tip |
| 18 | First tip surface facing axially inwards |
| 19 | Radial cutting channel |
| 20 | Second tip |
| 21 | Second proximal attachment root of the second tip, or second tip attachment root |
| 22 | Second distal free end of the second tip, or free end of the second tip |
| 23 | Second tip connecting stem |
| 24 | Second tip counter-blade portion |
| 25 | Second termination seat of the second tip |
| 26 | Second through hole of the second root of the second tip |
| 27 | Second tip interface cutting portion |
| 27.0 | Proximal free end of the cutting interface portion |
| 27.1 | First proximal arm of the cutting interface portion |
| 27.2 | Second distal arm of the cutting interface portion |
| 28 | Axial deformation seat of the second tip, or Axial deformation seat of the counter- |
| | blade |
| 29 | Radial cutting channel |
| 30 | Blade link |
| 31 | Proximal attachment root of the blade link, or blade link root |
| 32 | Distal blade link end |
| 33 | Transverse blade link bridge |
| 34 | Blade portion cutting edge |
| 35 | Blade portion surface facing axially inwards |
| 36 | Hole edge of the first through hole of the blade link |
| 37 | Blade link drag engagement portion |
| 37.0 | Blade link drag seat inlet opening |
| 37.1 | Blade link closing drag surface |
| 37.2 | Blade link opening drag surface |
| 37.3 | Blade link opening drag leg |
| 37.4 | Cantilevered blade link ear |
| 38 | Arc surface of the hole edge of the blade link root |
| 39 | Blade link root cutting channel |
| 40 | Counter-blade link |
| 41 | Proximal attachment root of the counter-blade link, or counter-blade link root |
| 42 | Distal counter-blade link end |
| 43 | Surface facing axially inwards of the counter-blade holder link recess |
| 44 | Axial deformation seat for the blade of the blade holder link |
| 45 | Axial counter-blade link recess |
| 46 | Counter-blade link support surface |
| 47 | Second tip link drag seat |
| 47.0 | Counter-blade link drag ear |
| 47.1 | Counter-blade link closing drag surface |
| 47.2 | Counter-blade link opening drag surface |
| 48 | Thread-stop wall |
| 48.1 | Thread-stop wall recess |
| 49 | Radial cutting channel of the counter-blade link |
| 50 | Blade holder link |
| 51 | Proximal attachment root of the blade holder link, or blade holder link root |
| 52 | Distal blade holder link end |
| 54 | Fist tip closing stroke end surface |
| 55 | First tip body connecting portion, or first connecting portion |
| 57 | Blade holder link drag engagement portion |
| 57.0 | Blade holder link drag tooth |
| 57.1 | Blade holder link closing drag surface |
| 57.2 | Blade holder link opening drag surface |
| 58 | Surface facing axially inwards of the blade holder link |
| 60 | Counter-blade holder link |
| 61 | Proximal attachment root of the counter-blade holder link |
| 63 | Second tip gripping surface, or second gripping surface |
| 64 | Counter-blade cutting edge |
| 65 | Second tip body connecting portion, or second connecting portion |
| 66 | Notch |
| 67 | Counter-blade holder link drag seat |
| 67.1 | Counter-blade holder link closing drag surface |
| 67.2 | Counter-blade holder link opening drag surface |
| 68 | Suture thread |
| 69 | Elastic leg |
| 70 | Tendon termination |
| 71 | First tip opening actuation tendon |
| 72 | First tip closing actuation tendon |
| 73 | Second tip opening actuation tendon |
| 74 | Second tip closing actuation tendon |
| 75 | Support link actuation tendon |
| 76 | Support link actuation counter-tendon |
| 77 | Cantilevered drag leg of the first termination seat of the first tip |
| 77.1 | Free end of the leg |
| 78 | Cantilevered drag leg of the second termination seat of the second tip |
| 78.1 | Free end of the leg |
| 79 | Ruled pulley surface of the first tip |
| 80 | Ruled pulley surface of the second tip |
| 81 | First external contact surface of the first root, or external contact surface of the first |
| | root |
| 82 | Second external contact surface of the second root, or external contact surface of |
| | the second root |
| 83 | First internal contact surface of the first root, or internal contact surface of the first |
| | root |
| 84 | Second internal contact surface of the second root, or internal contact surface of the |
| | second root |
| 85 | Blade link contact surface |
| 86 | Blade holder link contact surface |
| 87 | Internal contact counter-surface of the first prong, or first internal contact counter- |
| | surface of the first prong |
| 88 | Internal contact counter-surface of the second prong, or second internal contact |
| | counter-surface of the second prong |
| 89.1 | Counter-blade link contact surface |
| 89.2 | Counter-blade holder link contact surface |
| 90 | Connection link |
| 91 | First prong of the connection link |
| 92 | Second prong of the connection link |
| 93 | Proximal articulation pin |
| 94 | Fixing device |
| 96 | Convex ruled surface of the support link |
| 97 | Convex ruled surface of the connection link |
| 98 | Convex ruled surface of the support link |
| 99 | Convex ruled surface of the connection link |
| 101 | Robotic surgery system |
| 102 | Proximal shaft end |
| 103 | Robotic manipulator |
| 104 | Proximal interface portion of the surgical instrument, or surgical instrument backend |
| | portion |
| 105 | Positioning arm |
| 106 | Support portion, or cart, or tower |
| 107 | Master console |
| 108 | Master control device |
| 200 | Wire electro-erosion machine |
| 202 | Cutting wire |
| 204 | Workpiece which will form the blade portion |
| 205 | Flap |
| 206 | Lower head of the electro-erosion machine |
| 207 | Upper head of the electro-erosion machine |
| 208 | Electro-erosion machine tank |
| 209 | Electro-erosion machine reel |
| 210 | Workpiece thickness |
| 211 | Conduit |
| 212 | Pump |
| 213 | Nozzle |
| 214 | Jig or fixture |
| 215 | Jig fixing portion |
| 216 | Electro-erosion machine bracket |
| 217 | First rotatable portion for housing the jig |
| 218 | Jig or fixture motor |
| 219 | Screws |
| 220 | Jig stroke end |
| 221 | Rectified jig surface |
| 222 | Rectified jig surface |
| 223 | Workpiece cutting wall |
| 224 | Workpiece back face |
| 225 | Workpiece front face |
| 228 | Surface treatment, e.g., coating and/or heat treatment |
| 229 | Calibration reference point |
| 230 | Shaping cutting trajectory or path |
| 231 | Connection bridge |
| 232 | Starting point of a shaping cutting trajectory or path pass |
| 233 | Ending point of a shaping cutting trajectory or path pass |
| 234 | Edge to be machined |
| 235 | Starting point of a sharpening cutting trajectory or path pass |
| 236 | Ending point of a sharpening cutting trajectory or path pass |
| 238 | External cutting profile section |
| 239 | Cutting profile notch |
| 240 | Sharpening cutting trajectory or path |
| 241 | Longitudinal slot of the jig housing portion |
| 242 | Control system |
| 243 | Bowl |
| 250 | Semi-finished product |
| 260 | Press |
| 264 | Press hammer |
| 262 | Press anvil |
| 270 | Second rotatable portion for housing the jig |
| 302 | Piece to be shaped which will form the support structure, e.g., the connection link |
| 304 | Second workpiece |
| 320 | Piece to be shaped which will form the second tip link |
| 350 | Piece to be shaped which will form the blade holder link |
| 390 | Piece to be shaped which will form the connection link |
| 502 | Distal rotational joint |
| 509 | Proximal rotational joint |
| α | Sharpening rotation angle |
| β | Acute angle of the sharp edge |
| X-X | Longitudinal shaft or rod axis |
| Y-Y | Common rotation axis, or common distal rotation axis or common yaw rotation axis |
| P-P | Common proximal rotation axis, or common pitch rotation axis |
| F-F | Jig rotation axis |
| W | Cutting wire feeding direction or cutting direction |
| Y | Degree of freedom of yaw |
| P | Degree of freedom of pitch |
| G | Opening/closing direction, or degree of freedom of cutting |
| R | Degree of freedom of roll |
| POC | At least one point of contact between blade and counter-blade |
| D1 | First tip back side |
| P1 | First tip cutting side |
| D2 | Second tip back side |
| P2 | Second tip cutting side |
| Y5 | Axial distance |
| Y5′ | Axial distance |
| Y8 | Axial distance |

1. A surgical instrument for a robotic surgery system comprising an articulated end-effector comprising:
a support structure;
a first tip comprising a first proximal attachment root and a first distal free end;
a second tip comprising a second proximal attachment root and a second distal free end;
wherein:
the support structure, the first tip and the second tip are mutually articulated in a common rotation axis defining an axial direction coincident with or parallel to the common rotation axis, defining a relative degree of freedom of opening/closing between the first tip and the second tip;
the first root of the first tip and the second root of the second tip are axially proximate the support structure; and
wherein:
said first tip comprises a blade portion with a cutting edge integral in rotation with the first free end;
said blade portion of the first tip is elastically bendable in the axial direction;
said second tip comprises a counter-blade portion integral in rotation with the second free end;
said counter-blade portion is adapted to abut against said cutting edge by axially elastically bending said blade portion of the first tip, so that said cutting edge of the first tip and said counter-blade portion of the second tip reach a mechanical interference contact condition to exert a cutting action;
the first root of the first tip is in direct and intimate contact with the support structure and the second root of the second tip is in direct and intimate contact with the support structure.

2. The surgical instrument according to claim 1, wherein
the first root of the first tip and the second root of the second tip are articulated to the support structure about said common rotation axis defining a degree of freedom of orientation between the support structure and the assembly formed by said first tip and said second tip.

3. The surgical instrument according to claim 1,
wherein said first root and said second root are jointly interposed between the support structure; and/or
wherein:
said first root of the first tip comprises a first axially facing external contact surface and said support structure comprises a first prong comprising a first axially facing internal contact counter-surface,
said second root of the second tip comprises a second axially facing external contact surface and said support structure comprises a second prong comprising a second axially facing internal contact counter-surface;
said first external contact surface of the first root, said first internal contact counter-surface of the first prong, said second external contact surface of the second root, and said second internal contact counter-surface of the second prong are all parallel to one another.

4. The surgical instrument according to claim 1, wherein said counter-blade portion of the second tip protrudes axially for bending the first tip; and wherein said counter-blade portion.

5. The surgical instrument according to claim 1, wherein the body of the counter-blade portion of the second tip is elastically bendable in the axial direction.

6. The surgical instrument according to claim 5, wherein
the body of the second tip comprises a proximal cantilevered arm being elastically deformable axially outwards and having a proximal free end, and a proximal portion of the counter-blade portion belongs to said proximal cantilevered arm.

7. The surgical instrument according to claim 1, wherein at least one of the first tip and the second tip comprises an axial deformation seat forming an axial recess for housing the elastic deformation of the blade portion or the counter-blade portion during the cutting action.

8. The surgical instrument according t claim 1, wherein:
said first root of the first tip comprises a first through hole, and said second root of the second tip-comprises a second through hole,
said first through hole of the first root and said second through hole of the second root are all circular through holes coaxial to said common rotation axis and receive a single articulation pin extending in the direction of the common rotation axis.

9. The surgical instrument according to claim 1, wherein the body of the first tip is formed by two separate pieces, or links, comprising:
a blade link having a body comprising in a single piece said blade portion with said cutting edge and a blade link root, and
a blade holder link having a blade holder link root, and
wherein the blade link root and the blade holder link root are next to and in direct and intimate contact with each other, jointly forming said first root of the first tip.

10. The surgical instrument according to claim 9, further comprising a rotational drag engagement between said blade link and said blade holder link of the first tip which is placed distally with respect to the first root of the first tip.

11. The surgical instrument according to claim 9, further comprising a closing stroke end for said blade link which is placed distally with respect to the first root of the first tip.

12. The surgical instrument according to claim 9, wherein said blade link root is axially interposed between said blade holder link root and the second root of the second tip and in direct and intimate contact with said blade holder link root and the second root of the second tip.

13. The surgical instrument according to claim 1, wherein the first root of the first tip comprises, integral in rotation with said blade portion, a first termination seat for at least one actuation tendon of the first tip about said common rotation axis,
the second root of the second tip comprises, integral in rotation with said counter-blade portion, at least a second termination seat for at least one actuation tendon of the second tip about said common rotation axis.

14. The surgical instrument according to claim 1, wherein the blade portion is sharpened by wire electro-erosion.

15. A robotic surgery system comprising at least one surgical instrument according to claim 1.

16. A rotational joint of a cutting joint actuated by actuation tendons having a rotation axis, comprising:
a support structure,
a first attachment root integral in rotation with a first free end and with a blade portion having a cutting edge and having an elastically bendable body in the axial direction,
a second attachment root integral in rotation with a second free end and with a counter-blade portion; wherein:
the cutting edge of the blade portion is adapted to abut against said counter-blade portion during the movement of the degree of freedom of opening/closing of the cutting joint in a mechanical interference contact condition to exert a cutting action;
the first root of the first tip is in direct and intimate contact with the support structure and the second root of the second tip is in direct and intimate contact with the support structure.

17. The surgical instrument according to claim 1, wherein said counter-blade portion of the second tip protrudes axially for bending the first tip; and wherein said counter-blade portion is a curved protruding surface having a concavity facing axially inwards.

18. The surgical instrument according to claim 1, wherein the body of the counter-blade portion of the second tip is elastically bendable axially outwards.

19. The surgical instrument according to claim 5, wherein the body of the second tip comprises a proximal cantilevered arm being elastically deformable axially outwards and having a proximal free end, and a proximal portion of the counter-blade portion (24) belongs to said proximal cantilevered arm; and wherein the surgical instrument is capable of performing a cutting action for opening angles of the degree of freedom of opening/closing up to 60°.

20. The surgical instrument according to claim 9, further comprising a rotational drag engagement between said blade link and said blade holder link of the first tip which is placed distally with respect to the first root of the first tip, and is placed along the longitudinal extension of the blade portion.
